(19) United States
(12) Patent Application Publication
(10) Pub. No.: US 2006/0116366 A1
(43) Pub. Date: Jun. 1, 2006

(54) Spirocyclic ureas, compositions containing such compounds and methods of use

(76) Inventors: Emma R. Parmee, Scotch Plains, NJ (US); Fengqi Zhang, Edison, NJ (US); Dong-Ming Shen, Edison, NJ (US); John Stelmach, Westfield, NJ (US)

Correspondence Address: MERCK AND CO., INC, P O BOX 2000, RAHWAY, NJ, 07065-0907, US

(21) Appl. No.: 10/537,187

(22) PCT Filed: Nov. 26, 2003
(86) PCT No.: PCT/US03/38590

§ 371 Date: Jun. 2, 2005

Related U.S. Application Data
(60) Provisional application No. 60/430,799, filed on Dec. 4, 2002.

(51) Int. Cl. A61K 31/551; A61K 31/519

(52) U.S. Cl. 514/221; 540/500; 544/231; 514/257

(57) ABSTRACT

The present invention relates to spirocyclic ureas, compositions containing such compounds and methods of treatment The compounds are glucagon receptor antagonists and thus are useful for treating, preventing or delaying the onset of type 2 diabetes mellitus.

### BACKGROUND OF THE INVENTION

The present invention relates to spirocyclic urea derivatives, compositions containing such compounds and methods of treating type 2 diabetes mellitus.

Diabetes refers to a disease process derived from multiple causative factors and is characterized by elevated levels of plasma glucose (hyperglycemia) in the fasting state or following glucose administration during an oral glucose tolerance test. Frank diabetes mellitus (e.g., a blood glucose level≧126 mg/dL in a fasting state) is associated with increased and premature cardiovascular morbidity and mortality, and is related directly and indirectly to various metabolic conditions, including alterations of lipid, lipoprotein and apolipoprotein metabolism.

Patients with non-insulin dependent diabetes mellitus (type 2 diabetes mellitus), approximately 95% of patients with diabetes mellitus, frequently display elevated levels of serum lipids, such as cholesterol and triglycerides, and have poor blood-lipid profiles, with high levels of LDL-cholesterol and low levels of HDL-cholesterol. Those suffering from Type 2 diabetes mellitus are thus at an increased risk of developing macrovascular and microvascular complications, including coronary heart disease, stroke, peripheral vascular disease, hypertension (for example, blood pressure≧130/80 mmHg in a resting state), nephropathy, neuropathy and retinopathy.

Patients having type 2 diabetes mellitus characteristically exhibit elevated plasma insulin levels compared with nondiabetic patients; these patients have developed a resistance to insulin stimulation of glucose and lipid metabolism in the main insulin-sensitive tissues (muscle, liver and adipose tissues). Thus, Type 2 diabetes, at least early in the natural progression of the disease is characterized primarily by insulin resistance rather than by a decrease in insulin production, resulting in insufficient uptake, oxidation and storage of glucose in muscle, inadequate repression of lipolysis in adipose tissue, and excess glucose production and secretion by the liver. The net effect of decreased sensitivity to insulin is high levels of insulin circulating in the blood without appropriate reduction in plasma glucose (hyperglycemia). Hyperinsulinemia is a risk factor for developing hypertension and may also contribute to vascular disease.

Glucagon serves as the major regulatory hormone attenuating the effect of insulin in its inhibition of liver gluconeogenesis and is normally secreted by α-cells in pancreatic islets in response to falling blood glucose levels. The hormone binds to specific receptors in liver cells that triggers glycogenolysis and an increase in gluconeogenesis through cAMP-mediated events. These responses generate glucose (e.g. hepatic glucose production) to help maintain euglycemia by preventing blood glucose levels from falling significantly.

In addition to elevated levels of circulating insulin, type II diabetics have elevated levels of plasma glucagon and increased rates of hepatic glucose production. Antagonists of glucagon are useful in improving insulin responsiveness in the liver, decreasing the rate of gluconeogenesis and lowering the rate of hepatic glucose output resulting in a decrease in the levels of plasma glucose.

### SUMMARY OF THE INVENTION

The present invention is directed to a compound represented by formula I: 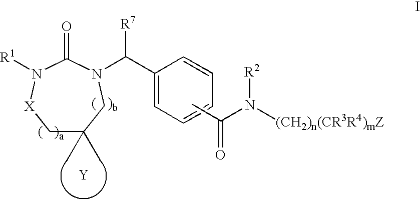
or a pharmaceutically acceptable salt or solvate thereof, wherein:
a and b are independently selected from the integers 0 and 1, such that the sum of a and b is 0 or 1;

X is selected from CH₂ and C(O);

R¹ is selected from the group consisting of:

(1) C₁₋₁₅ alkyl optionally substituted with up to five groups as follows: (a) 1-3 OH groups; (b) 1 oxo group; (c) 1-5 halo groups, up to a perhaloalkyl group; (d) 1-3 C₁₋₆ alkoxy groups optionally substituted with up to five halo or a perhaloalkoxy, or up to 2 hydroxy or CO₂R⁶ groups; (e) 1-2 CO₂R⁶ groups and (f) 1-2 phenyl groups, each optionally substituted as follows: 1-5 halo groups, (2) 1-2 OH, CO₂R⁶, CN or S(O)ₚR⁵ groups, and (3) 1-2 C₁₋₆ alkyl or alkoxy groups, each optionally substituted with 1-5 halo, up to perhaloalkyl, and 1-2 OH or CO₂R⁶ groups; and

(2) aryl or heteroaryl, optionally substituted as set forth below:
(a) 1-3 hydroxy groups; (b) 1-5 halo groups; (c) 1-3 C₁₋₁₅ alkyl or alkoxy groups, each optionally substituted with up to five halo and 1-2 hydroxy or CO₂R⁶ groups; (d) 1-2 CO₂R⁶, CN, S(O)ₚR⁵ or CONR⁹R¹⁰ groups; (e) NR⁹R¹⁰; (f) SCF₃; (g) phenyl, heteroaryl or O-phenyl, said group being optionally substituted with 1-5 halo groups, 1-2 OH, CO₂R⁶, CN or S(O)ₙR⁵ groups, and 1-2 C-6 alkyl or alkoxy groups, each optionally substituted with 1-5 halo, up to perhaloalkyl, and 1-2 OH or CO₂R⁶ groups;

R² represents H or C₁₋₆alkyl;

R³ represents H or F;

R⁴ is selected from the group consisting of H, F and OH;

or R³ and R⁴ are taken in combination and represent an oxo group;

R⁵ represents a C₁₋₁₀alkyl group;

R⁶ represents H or C₁₋₁₀alkyl, optionally substituted with OH, OC₁₋₆alkyl, CO₂H, CO₂C₁₋₆alkyl, and 1-3 halo groups;

R⁷ represents H, CO₂R⁶, C₁₋₆alkyl optionally substituted with OH, OC₁₋₆alkyl, CO₂R⁶ or 1-3 halo groups;

R⁸ and R⁹ are independently selected from H and C₁₋₆alkyl;

R¹⁰ is H or is independently selected from:

(a) C₁₋₁₀alkyl, optionally substituted with OH, OC₁₋₆alkyl, CO₂H, CO₂C₁₋₆alkyl, and 1-3 halo groups; (b) aryl or C₁₋₆ alkaryl, each optionally substituted with 1-5 halos and 1-3 members selected from the group consisting of: CN, OH, C₁₋₁₀alkyl and OC₁₋₁₀alkyl, said alkyl and alkoxy being further optionally substituted with 1-5 halo groups up to perhalo; (c) heterocycle, or C₁₋₆alkyl-heterocycle, optionally substituted with 1-5 halo groups and 1-3 groups selected from: oxo, C₁₋₁₀alkyl and OC₁₋₁₀ alkyl, said alkyl and alkoxy being further optionally substituted with 1-5 halo groups up to perhalo; and (d) heteroaryl or C₁₋₆alkyl-heteroaryl, optionally substituted with 1-5 halo groups and 1-3 groups selected from: C₁₋₁₀alkyl and OC₁₋₁₀ alkyl, said alkyl and alkoxy being further optionally substituted with 1-5 halo groups up to perhalo;

R¹¹ is independently selected from the group consisting of:

(a) C₁₋₁₀alkyl, optionally substituted with OH, OC₁₋₆alkyl, CO₂H, CO₂C₁₋₆alkyl, and 1-3 halo groups; (b) aryl or C₁₋₆ alkaryl, each optionally substituted with 1-5 halos and 1-3 members selected from the group consisting of: CN, OH, C₁₋₁₀alkyl and OC₁₋₁₀ alkyl, said alkyl and alkoxy being further optionally substituted with 1-5 halo groups up to perhalo; (c) heterocycle, or C₁₋₆alkyl-heterocycle, optionally substituted with 1-5 halo groups and 1-3 groups selected from: oxo, C₁₋₁₀alkyl and OC₁₋₁₀ alkyl, said alkyl and alkoxy being further optionally substituted with 1-5 halo groups up to perhalo; and (d) heteroaryl or C₁₋₆alkyl-heteroaryl, optionally substituted with 1-5 halo groups and 1-3 groups selected from: C₁₋₁₀alkyl and OC₁₋₁₀ alkyl, said alkyl and alkoxy being further optionally substituted with 1-5 halo groups up to perhalo;

Y represents a 4 to 8 membered spirocarbocyclic ring or a spiroheterocyclic ring containing up to three heteroatoms, 0-1 of which are selected from O and S and 0-3 of which are N,

said spirocarbocyclic or spiroheterocyclic ring being optionally substituted on either carbon or nitrogen atoms with up to three groups independently selected as follows:

(a) 1-2 phenyl groups, each being optionally substituted with one to five groups independently selected from the group consisting of: (1) 1-3 hydroxy groups; (2) 1-5 halo groups; (3) 1-3 C₁₋₈alkyl or alkoxy groups, each being further optionally substituted with 1-5 halo or 1-2 OH or CO₂R⁶ groups, and (4) 1-2 CO₂R⁶, CN, S(O)ₚR⁵, CONR⁹R¹⁰ or NO₂ groups;

(b) C₁₋₁₀ alkyl optionally substituted with 1-5 groups selected as follows: (i) 1-3 hydroxy groups; (ii) 1 oxo group; (iii) 1-5 halo groups up to perhalo; (iv) 1-3 C₁₋₁₀ alkoxy groups, optionally substituted with 1-5 halo groups up to perhalo, or 1-2 hydroxy or CO₂R⁶ groups; (v) 1-2 CO₂R⁶ groups; (vi) phenyl, optionally substituted with one to five groups independently selected from the group consisting of: (a) 1-3 hydroxy groups; (b) 1-5 halo groups; (c) 1-3 C₁₋₆ alkyl or alkoxy groups, optionally substituted with 1-5 halo groups up to perhalo, or 1-2 hydroxy or CO₂R⁶ groups; (d) 1-2 CO₂R⁶, CN, S(O)ₚR⁵, CONR⁹R¹⁰ or NO₂ groups; (e) 1-2 phenyl rings, each of which is optionally substituted as follows: 1-3 C₁₋₁₀ alkyl or alkoxy groups, each being further optionally substituted with 1-5 halo up to perhalo, or 1-2 hydroxy or CO₂R⁶ groups;

said spirocarbocyclic or spiroheterocyclic ring being further optionally substituted on a carbon atom with a member selected from the group consisting of:

(a) —NR⁸—C(O)—NR⁹R¹⁰; (b) —NR⁸—CO₂R¹¹; (c) —NR⁸—C(O)R¹¹; (d) —NR⁹R¹⁰;

(e) —NR⁸SO₂R¹¹; (f) —SO₂—NR⁹R¹⁰; (g) —C(O)NR⁹R¹⁰ and (h) —OC(O)—NR⁹R¹⁰;

and when said ring contains a nitrogen atom, said ring being further optionally substituted on the nitrogen atom with a member selected from the group consisting of:

(a) —C(O)NR⁹R¹⁰; (b) —CO₂R¹¹; (c) C(O)R¹¹; and (d) —SO₂R¹¹;

m and p are independently selected from 0, 1 and 2, and n is an integer from 0 to 6,

when both m and n are zero, Z is selected from 5-tetrazolyl and 5-(2-oxo-1,3,4-oxadiazolyl) and when one of m and n is other than zero, Z is selected from the group consisting of: CO₂R⁶, with R⁶ as defined above, 5-tetrazolyl and 5-(2-oxo-1,3,4-oxadiazolyl).

### DETAILED DESCRIPTION OF THE INVENTION

All references cited herein are incorporated by reference. The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-10 carbon atoms are intended for linear or branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-10 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” (Ar) means mono- and bicyclic aromatic rings containing 6-12 carbon atoms. Examples of aryl include phenyl, naphthyl, indenyl and the like.

“Heteroaryl” (HAR) means a mono- or bicyclic aromatic ring or ring system containing at least one heteroatom selected from O, S and N, with each ring containing 5 to 6 atoms. Examples include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl and the like. Heteroaryl also includes aromatic heterocyclic groups fused to heterocycles that are non-aromatic or partially aromatic, and aromatic heterocyclic groups fused to cycloalkyl rings.

“Heterocyclyl” (Hetcy) means mono- and bicyclic saturated rings and ring systems containing at least one heteroatom selected from N, S and O, each of said ring having from 3 to 10 atoms in which the point of attachment may be carbon or nitrogen. Examples of “heterocyclyl” include pyrrolidinyl, piperidinyl, piperazinyl, imidazolidinyl, 2,3-dihydrofuro(2,3-b)pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

“Halogen” (Halo) includes fluorine, chlorine, bromine and iodine.

A first aspect of the invention is directed to a compound represented by formula I: 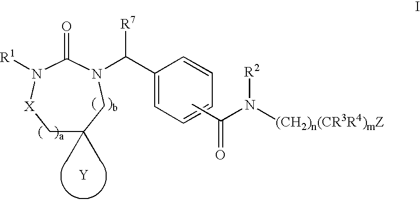
or a pharmaceutically acceptable salt or solvate thereof, wherein:
a and b are independently selected from the integers 0 and 1, such that the sum of a and b is 0 or 1;

X is selected from CH₂ and C(O);

R¹ is selected from the group consisting of:

(1) C₁₋₁₅ alkyl optionally substituted with up to five groups as follows: (a) 1-3 OH groups; (b) 1 oxo group; (c) 1-5 halo groups, up to a perhaloalkyl group; (d) 1-3 C₁₋₆ alkoxy groups optionally substituted with up to five halo or a perhaloalkoxy, or up to 2 hydroxy or CO₂R⁶ groups; (e) 1-2 CO₂R⁶ groups and (f) 1-2 phenyl groups, each optionally substituted as follows: 1-5 halo groups, (2) 1-2 OH, CO₂R⁶, CN or S(O)ₚR⁵ groups, and (3) 1-2 C₁₋₆ alkyl or alkoxy groups, each optionally substituted with 1-5 halo, up to perhaloalkyl, and 1-2 OH or CO₂R⁶ groups; and

(2) aryl or heteroaryl, optionally substituted as set forth below:
(a) 1-3 hydroxy groups; (b) 1-5 halo groups; (c) 1-3 C₁₋₁₅ alkyl or alkoxy groups, each optionally substituted with up to five halo and 1-2 hydroxy or CO₂R⁶ groups; (d) 1-2 CO₂R⁶, CN, S(O)ₚR⁵ or CONR⁹R¹⁰ groups; (e) NR⁹R¹⁰; (f) SCF₃; (g) phenyl, heteroaryl or O-phenyl, said group being optionally substituted with 1-5 halo groups, 1-2 OH, CO₂R⁶, CN or S(O)ₙR⁵ groups, and 1-2 C₁₋₆ alkyl or alkoxy groups, each optionally substituted with 1-5 halo, up to perhaloalkyl, and 1-2 OH or CO₂R⁶ groups;

R² represents H or C₁₋₆alkyl;

R³ represents H or F;

R⁴ is selected from the group consisting of H, F and OH;

or R³ and R⁴ are taken in combination and represent an oxo group;

R⁵ represents a C₁₋₁₀alkyl group;

R⁶ represents H or C₁₋₁₀alkyl, optionally substituted with OH, OC₁₋₆alkyl, CO₂H, CO₂C₁₋₆alkyl, and 1-3 halo groups;

R⁷ represents H, CO₂R⁶, C₁₋₆alkyl optionally substituted with OH, OC₁₋₆alkyl, CO₂R⁶ or 1-3 halo groups;

R⁸ and R⁹ are independently selected from H and C₁₋₆alkyl;

R¹⁰ is H or is independently selected from:

(a) C₁₋₁₀alkyl, optionally substituted with OH, OC₁₋₆alkyl, CO₂H, CO₂C₁₋₆alkyl, and 1-3 halo groups; (b) aryl or C₁₋₆ alkaryl, each optionally substituted with 1-5 halos and 1-3 members selected from the group consisting of: CN, OH, C₁₋₁₀alkyl and OC₁₋₁₀ alkyl, said alkyl and alkoxy being further optionally substituted with 1-5 halo groups up to perhalo; (c) heterocycle, or C₁₋₆alkyl-heterocycle, optionally substituted with 1-5 halo groups and 1-3 groups selected from: oxo, C₁₋₁₀alkyl and OC₁₋₁₀ alkyl, said alkyl and alkoxy being further optionally substituted with 1-5 halo groups up to perhalo; and (d) heteroaryl or C₁₋₆alkyl-heteroaryl, optionally substituted with 1-5 halo groups and 1-3 groups selected from: C₁₋₁₀alkyl and OC₁₋₁₀ alkyl, said alkyl and alkoxy being further optionally substituted with 1-5 halo groups up to perhalo;

R¹¹ is independently selected from the group consisting of:

(a) C₁₋₁₀alkyl, optionally substituted with OH, OC₁₋₆alkyl, CO₂H, CO₂C₁₋₆alkyl, and 1-3 halo groups; (b) aryl or C₁₋₆ alkaryl, each optionally substituted with 1-5 halos and 1-3 members selected from the group consisting of: CN, OH, C₁₋₁₀alkyl and OC₁₋₁₀ alkyl, said alkyl and alkoxy being further optionally substituted with 1-5 halo groups up to perhalo; (c) heterocycle, or C₁₋₆alkyl-heterocycle, optionally substituted with 1-5 halo groups and 1-3 groups selected from: oxo, C₁₋₁₀alkyl and OC₁₋₁₀ alkyl, said alkyl and alkoxy being further optionally substituted with 1-5 halo groups up to perhalo; and (d) heteroaryl or C₁₋₆alkyl-heteroaryl, optionally substituted with 1-5 halo groups and 1-3 groups selected from: C₁₋₁₀alkyl and OC₁₋₁₀ alkyl, said alkyl and alkoxy being further optionally substituted with 1-5 halo groups up to perhalo;

Y represents a 4 to 8 membered spirocarbocyclic ring or a spiroheterocyclic ring containing up to three heteroatoms, 0-1 of which are selected from O and S and 0-3 of which are N,

said spirocarbocyclic or spiroheterocyclic ring being optionally substituted on either carbon or nitrogen atoms with up to three groups independently selected as follows:

(a) 1-2 phenyl groups, each being optionally substituted with one to five groups independently selected from the group consisting of: (1) 1-3 hydroxy groups; (2) 1-5 halo groups; (3) 1-3 C₁₋₈alkyl or alkoxy groups, each being further optionally substituted with 1-5 halo or 1-2 OH or CO₂R⁶ groups, and (4) 1-2 CO₂R⁶, CN, S(O)ₚR⁵, CONR⁹R¹⁰ or NO₂ groups;

(b) C₁₋₁₀ alkyl optionally substituted with 1-5 groups selected as follows: (i) 1-3 hydroxy groups; (ii) 1 oxo group; (iii) 1-5 halo groups up to perhalo; (iv) 1-3 C₁₋₁₀ alkoxy groups, optionally substituted with 1-5 halo groups up to perhalo, or 1-2 hydroxy or CO₂R⁶ groups; (v) 1-2 CO₂R⁶ groups; (vi) phenyl, optionally substituted with one to five groups independently selected from the group consisting of: (a) 1-3 hydroxy groups; (b) 1-5 halo groups; (c) 1-3 C₁₋₆ alkyl or alkoxy groups, optionally substituted with 1-5 halo groups up to perhalo, or 1-2 hydroxy or CO₂R⁶ groups; (d) 1-2 CO₂R⁶, CN, S(O)ₚR⁵, CONR⁹R¹⁰ or NO₂ groups; (e) 1-2 phenyl rings, each of which is optionally substituted as follows: 1-3 C₁₋₁₀ alkyl or alkoxy groups, each being further optionally substituted with 1-5 halo up to perhalo, or 1-2 hydroxy or CO₂R⁶ groups;

said spirocarbocyclic or spiroheterocyclic ring being further optionally substituted on a carbon atom with a member selected from the group consisting of:

(a) —NR⁸—C(O)—NR⁹R¹⁰; (b) —NR⁸—CO₂R¹¹; (c) —NR⁸—C(O)R¹¹; (d) —NR⁹R¹⁰;

(e) —NR⁸SO₂R¹¹; (f) —SO₂—NR⁹R¹⁰; (g) —C(O)NR⁹R¹⁰ and (h) —OC(O)—NR⁹R¹⁰;

and when said ring contains a nitrogen atom, said ring being further optionally substituted on the nitrogen atom with a member selected from the group consisting of:

(a) —C(O)NR⁹R¹⁰; (b) —CO₂R¹¹; (c) C(O)R¹¹; and (d) —SO₂R¹¹;

m and p are independently selected from 0, 1 and 2, and n is an integer from 0 to 6,

when both m and n are zero, Z is selected from 5-tetrazolyl and 5-(2-oxo-1,3,4-oxadiazolyl) and when one of m and n is other than zero, Z is selected from the group consisting of: CO₂R⁶, with R⁶ as defined above, 5-tetrazolyl and 5-(2-oxo-1,3,4-oxadiazolyl).

In another aspect of the invention that is of particular interest, a compound of formula I or a pharmaceutically acceptable salt or solvate thereof is disclosed wherein:

R¹ is selected from the group consisting of:

(1) C₁₋₆ alkyl optionally substituted with 1-3 groups selected from: OH, halo, C₁₋₃ alkoxy, halo-C₁₋₃alkoxy and phenyl, said phenyl being optionally substituted with 1-3 halo groups, SO₂R⁵, and 1-2 C₁₋₃alkyl or alkoxy groups optionally substituted with 1-3 halo groups, and

(2) aryl optionally substituted with 1-3 halo groups; 1-2 C₁₋₃alkyl or alkoxy groups, each optionally substituted with 1-3 halo groups; —NR⁹R¹⁰ wherein R⁹ and R¹⁰ are H or methyl; SCF₃ and heteroaryl. Within this aspect of the invention, all other variables are as originally defined.

In an aspect of the invention that is of even more particular interest, a compound of formula I or a pharmaceutically acceptable salt or solvate thereof is disclosed wherein R¹ represents phenyl optionally substituted with 1-2 groups selected from Br, Cl; trifluoromethyl and trifluoromethoxy. Within this aspect of the invention, all other variables are as originally defined.

In another aspect of the invention that is of interest, X represents CH₂. Within this aspect of the invention, all other variables are as originally defined.

In another aspect of the invention that is of interest, a and b represent 0 or a represents 1 and b represents 0. Within this aspect of the invention, all other variables are as originally defined.

In another aspect of the invention that is of particular interest, a compound of formula I or a pharmaceutically acceptable salt or solvate thereof is disclosed wherein Y represents a spiroC₄₋₈cycloalkyl group or a 5-6 membered spiroheterocyclic group containing 1 N atom,

said ring being optionally substituted with a Con alkyl group, which is optionally substituted with 1-3 halo groups or 1 Phenyl ring that is optionally substituted with 1-2 halo, 1-2 C₁₋₃ alkyl or alkoxy groups, said alkyl and alkoxy substituents being further optionally substituted with 1-3 halo groups. Within this aspect of the invention, all other variables are as originally defined.

In another aspect of the invention that is of more particular interest, a compound of formula I or a pharmaceutically acceptable salt or solvate thereof is disclosed wherein Y represents a spirocyclohexyl or spiropiperidinyl group that is substituted with a C₁₋₄ alkyl group that is optionally substituted with a phenyl ring. Within this aspect of the invention, all other variables are as originally defined.

In another aspect of the invention that is of even more particular interest, a compound of formula I or a pharmaceutically acceptable salt or solvate thereof is disclosed wherein Y represents a spirocyclohexyl group substituted with a t-butyl group at the 4 position. Within this aspect of the invention, all other variables are as originally defined.

In another aspect of the invention that is of particular interest, a compound of formula I or a pharmaceutically acceptable salt or solvate thereof is disclosed wherein R² is H or C₁₋₃alkyl. Within this subset, all other variables are as originally defined. More particularly, a compound of formula I is disclosed wherein R² represents H. Within this subset, all other variables are as originally defined.

In another aspect of the invention that is of particular interest, a compound of formula I or a pharmaceutically acceptable salt or solvate thereof is disclosed wherein R⁷ represents H or methyl. Within this aspect of the invention, all other variables are as originally defined. More particularly, a compound of formula I is disclosed wherein R⁷ represents H. Within this subset, all other variables are as originally defined.

In another aspect of the invention that is of particular interest, a compound of formula I or a pharmaceutically acceptable salt or solvate thereof is disclosed wherein n and m represent 0, and Z represents a 5-tetrazolyl group. Within this subset, all other variables are as originally defined.

In another aspect of the invention that is of particular interest, a compound of formula I or a pharmaceutically acceptable salt or solvate thereof is disclosed wherein m represents 0, n represents 2, and Z represents a CO₂R⁶ group. Within this subset, all other variables are as originally defined.

In another aspect of the invention that is of particular interest, a compound of formula I or a pharmaceutically acceptable salt or solvate thereof is disclosed wherein m and n each represent 1, R³ represents OH, R⁴ represents H and Z represents a CO₂R⁶ group. Within this subset, all other variables are as originally defined.

In another aspect of the invention that is of particular interest, a compound of formula I or a pharmaceutically acceptable salt or solvate thereof is disclosed wherein:

R¹ is selected from the group consisting of:

(1) C₁₋₆ alkyl optionally substituted with 1-3 groups selected from: OH, halo, C₁₋₃ alkoxy, halo-C₁₋₃alkoxy and phenyl, said phenyl being optionally substituted with 1-3 halo groups, SO₂R⁵, and 1-2 C₁₋₃alkyl or alkoxy groups optionally substituted with 1-3 halo groups, and

(2) aryl optionally substituted with 1-3 halo groups; 1-2 C₁₋₃alkyl or alkoxy groups, each optionally substituted with 1-3 halo groups; —NR⁹R¹⁰ wherein R⁹ and R¹⁰ are H or methyl; SCF₃ and heteroaryl;

X represents CH₂;

a and b represent 0 or a represents 1 and b represents 0;

Y represents a spiroC₄₋₈cycloalkyl group or a 5-6 membered spiroheterocyclic group containing 1 N atom,

said ring being optionally substituted with a C₁₋₆ alkyl group, which is optionally substituted with 1-3 halo groups or 1 Phenyl ring that is optionally substituted with 1-2 halo, 1-2 C₁₋₃ alkyl or alkoxy groups, said alkyl and alkoxy substituents being further optionally substituted with 1-3 halo groups;

R² is H or C₁₋₃alkyl;

R⁷ represents H or methyl;

m and n represent 0, and Z represents a 5-tetrazolyl group. Within this subset, all other variables are as originally defined.

In another aspect of the invention that is of particular interest, a compound of formula I is disclosed wherein:

R¹ is selected from the group consisting of:

(1) C₁₋₆ alkyl optionally substituted with 1-3 groups selected from: OH, halo, C₁₋₃ alkoxy, halo-C₁₋₃alkoxy and phenyl, said phenyl being optionally substituted with 1-3 halo groups, SO₂R⁵, and 1-2 C₁₋₃alkyl or alkoxy groups optionally substituted with 1-3 halo groups, and

(2) aryl optionally substituted with 1-3 halo groups; 1-2 C₁₋₃alkyl or alkoxy groups, each optionally substituted with 1-3 halo groups; —NR⁹R¹⁰ wherein R⁹ and R¹⁰ are H or methyl; SCF₃ and heteroaryl;

X represents CH₂;

a and b represent 0 or a represents 1 and b represents 0;

Y represents a spiroC₄₋₈cycloalkyl group or a 5-6 membered spiroheterocyclic group containing 1 N atom,

said ring being optionally substituted with a C₁₋₆ alkyl group, which is optionally substituted with 1-3 halo groups or 1 Phenyl ring that is optionally substituted with 1-2 halo, 1-2 C₁₋₃ alkyl or alkoxy groups, said alkyl and alkoxy substituents being further optionally substituted with 1-3 halo groups;

R² is H or C₁₋₃alkyl;

R⁷ represents H or methyl;

m represents 0, n represents 2, and Z represents a CO₂R⁶ group. Within this subset, all other variables are as originally defined.

In another aspect of the invention that is of particular interest, a compound of formula I is disclosed wherein:

R¹ is selected from the group consisting of:

(1) C₁₋₆ alkyl optionally substituted with 1-3 groups selected from: OH, halo, C₁₋₃ alkoxy, halo-C₁₋₃alkoxy and phenyl, said phenyl being optionally substituted with 1-3 halo groups, SO₂R⁵, and 1-2 C₁₋₃alkyl or alkoxy groups optionally substituted with 1-3 halo groups, and

(2) aryl optionally substituted with 1-3 halo groups; 1-2 C₁₋₃alkyl or alkoxy groups, each optionally substituted with 1-3 halo groups; —NR⁹R¹⁰ wherein R⁹ and R¹⁰ are H or methyl; SCF₃ and heteroaryl;

X represents CH₂;

a and b represent 0 or a represents 1 and b represents 0;

Y represents a spiroC₄₋₈cycloalkyl group or a 5-6 membered spiroheterocyclic group containing 1 N atom,

said ring being optionally substituted with a C₁₋₆ alkyl group, which is optionally substituted with 1-3 halo groups or 1 Phenyl ring that is optionally substituted with 1-2 halo, 1-2 C₁₋₃ alkyl or alkoxy groups, said alkyl and alkoxy substituents being further optionally substituted with 1-3 halo groups;

R² is H or C₁₋₃alkyl;

R⁷ represents H or methyl;

m and n each represent 1, R³ represents OH, R⁴ represents H and Z represents a CO₂R⁶ group. Within this subset, all other variables are as originally defined.

Species falling within the scope of the present invention that are of particular interest include the following:

TABLE 1

| Compound | Compound |
|---|---|
| 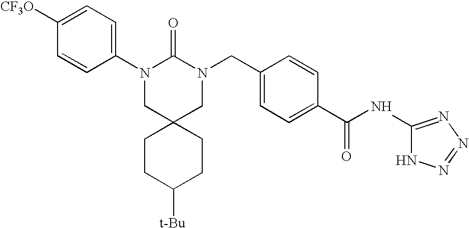 | 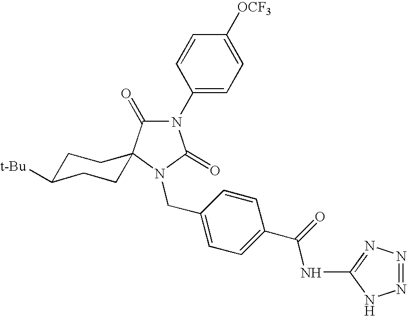 |
| 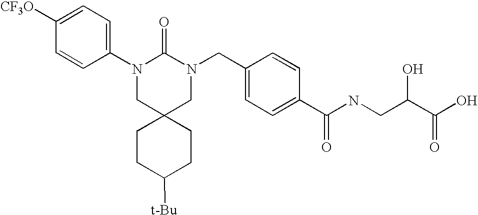 | 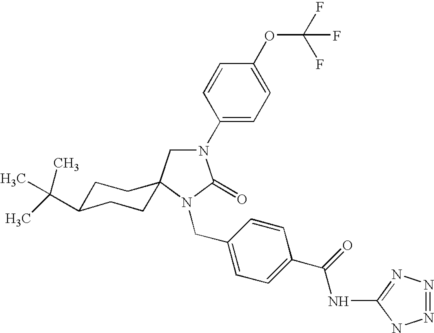 |
| 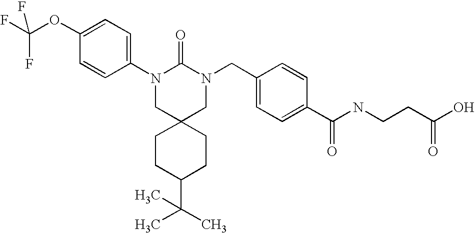 | 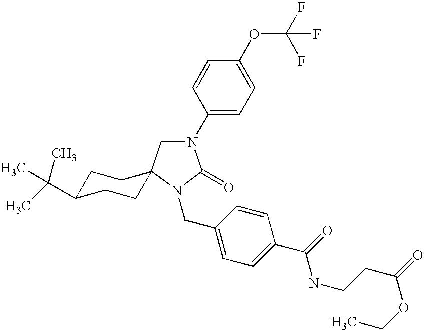 |
| 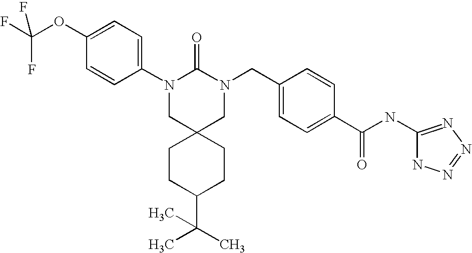 | 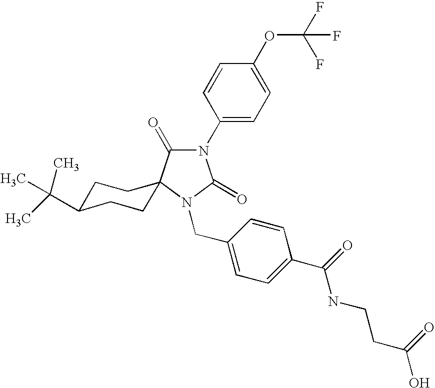 |
| 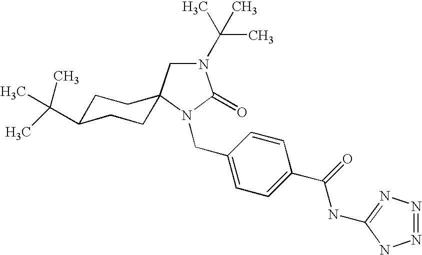 | 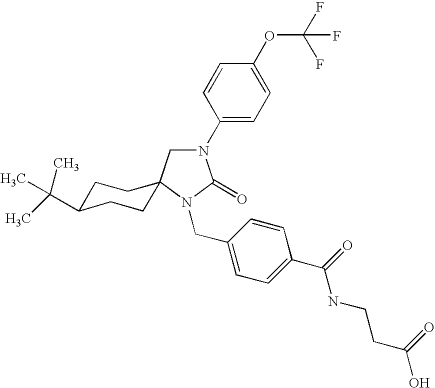 |
| 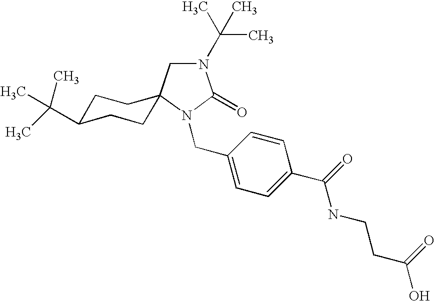 | 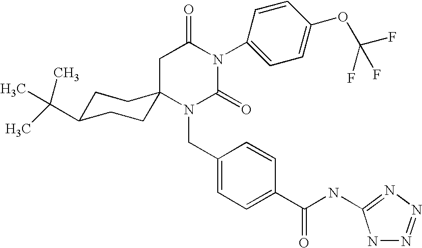 |
| 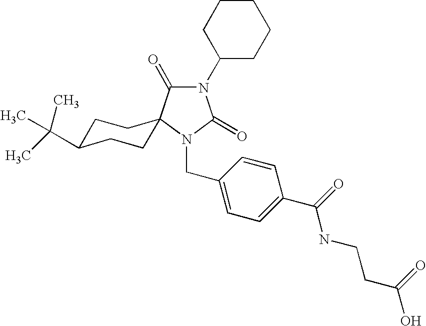 | 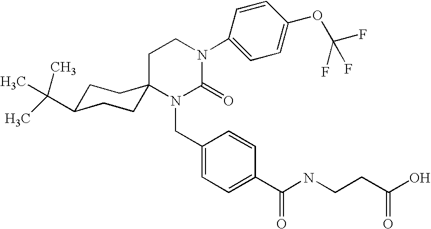 |
| 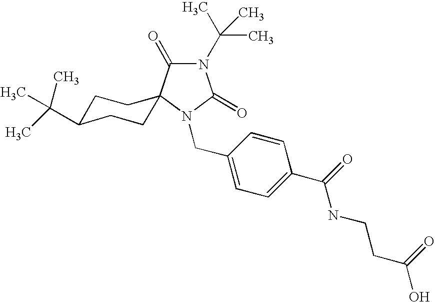 | 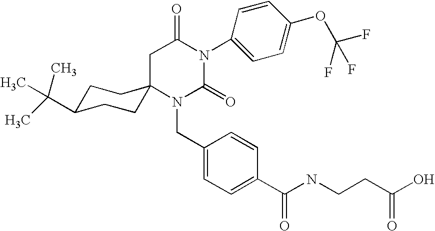 |
| 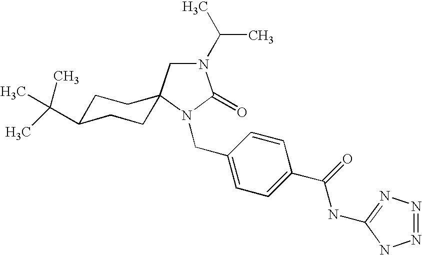 | 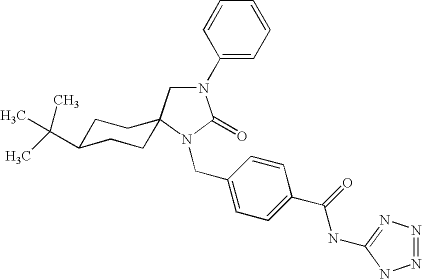 |
| 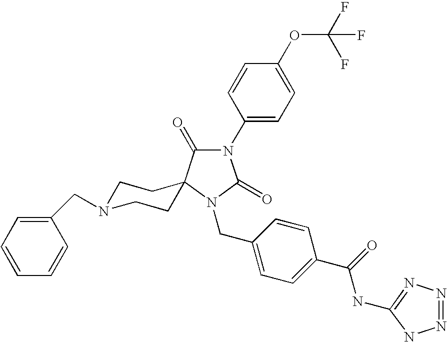 | 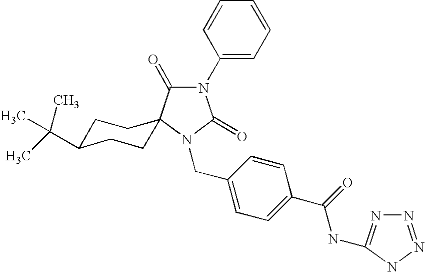 |
| 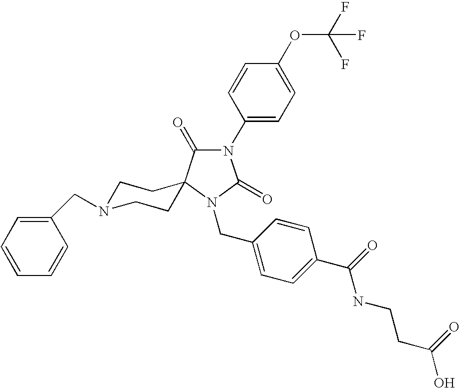 | 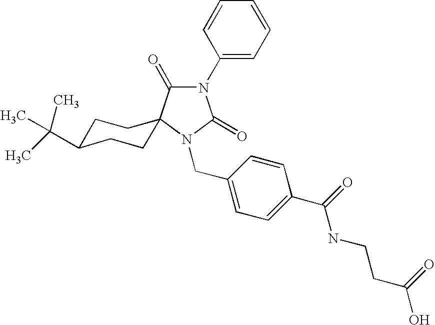 |
| 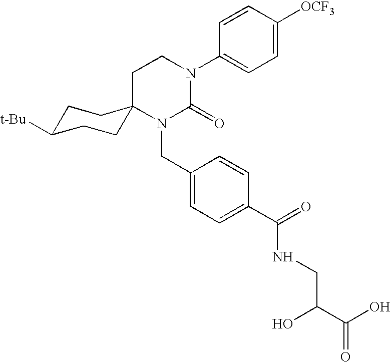 | 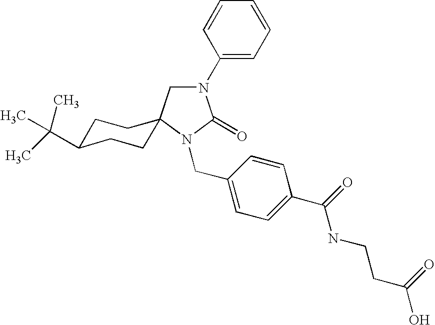 |
| 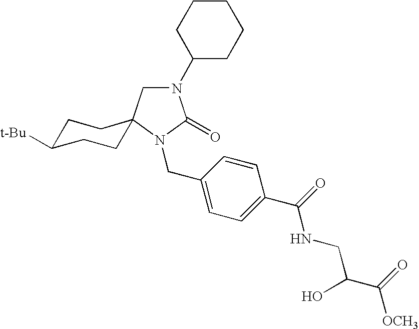 | 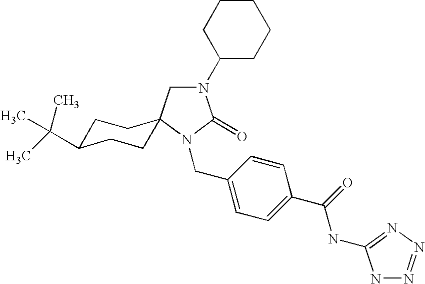 |
| 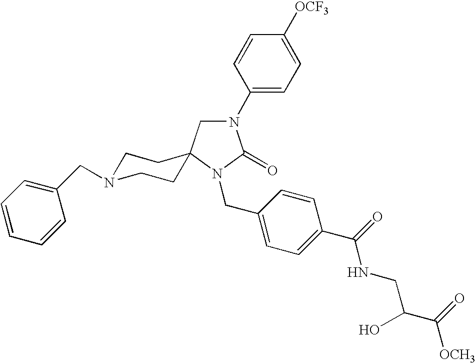 | 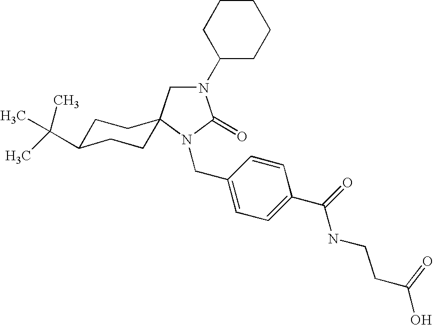 |
| 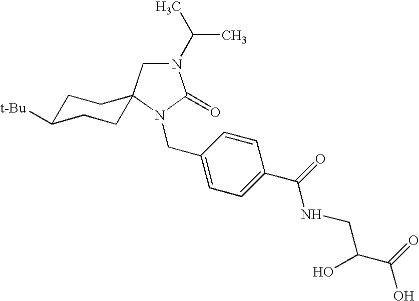 | 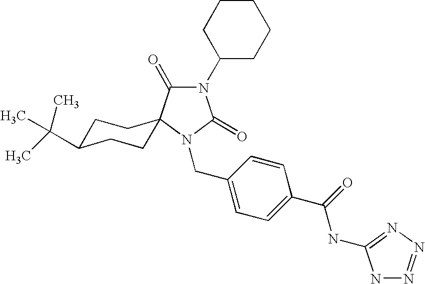 |
| 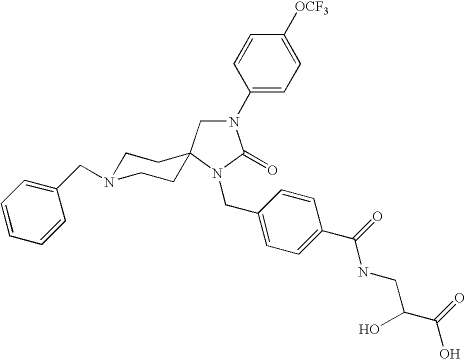 | 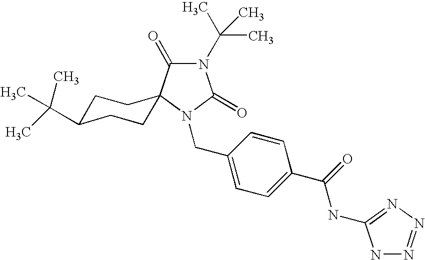 |
| 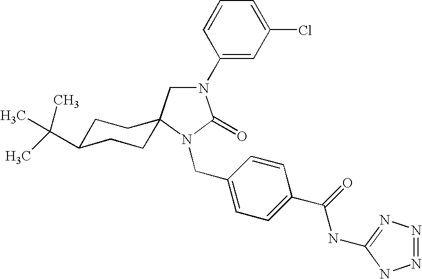 | 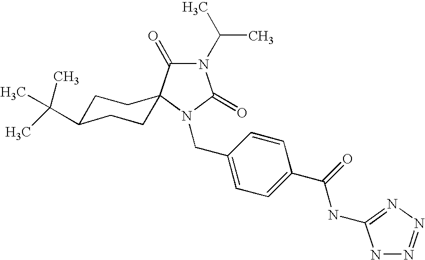 |
| 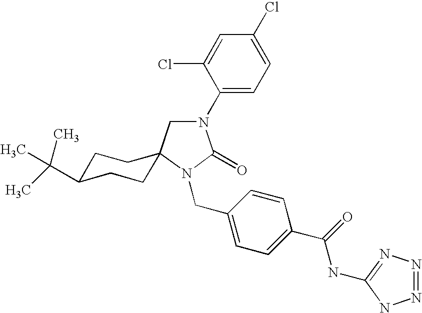 | 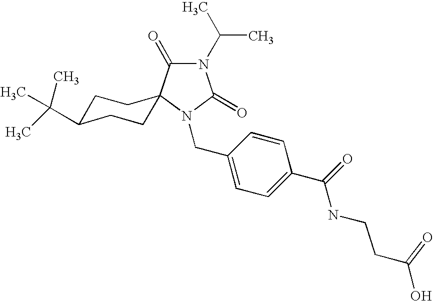 |
| 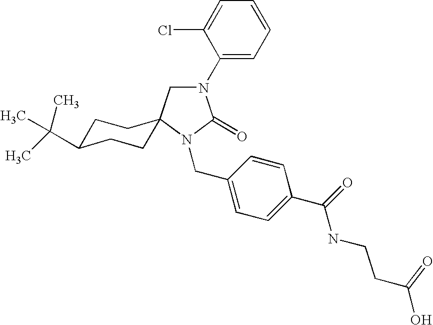 | 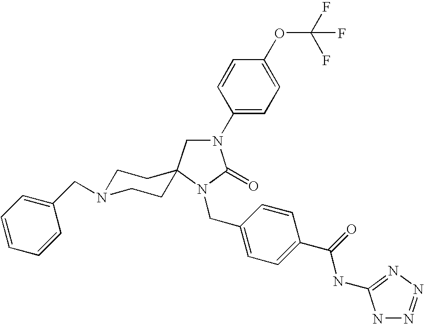 |
| 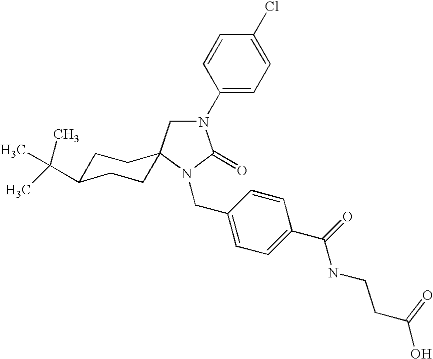 | 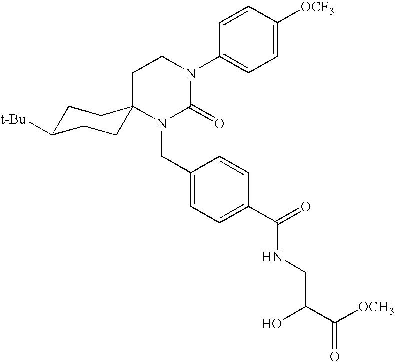 |
| 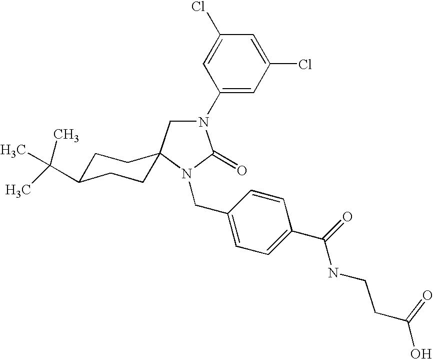 | 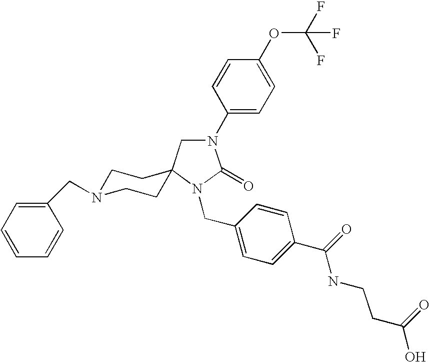 |
| 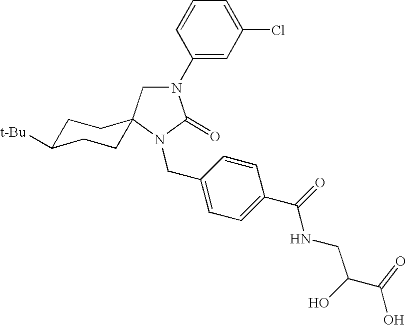 | 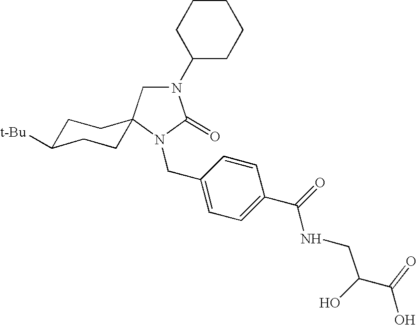 |
| 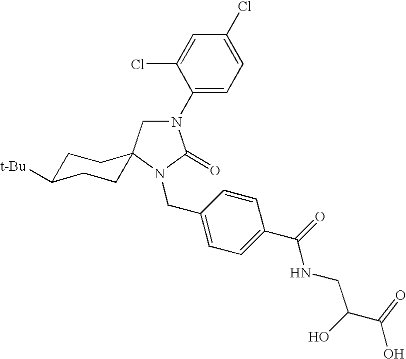 | 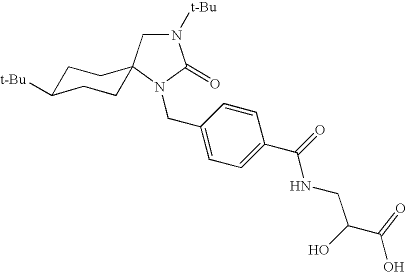 |
| 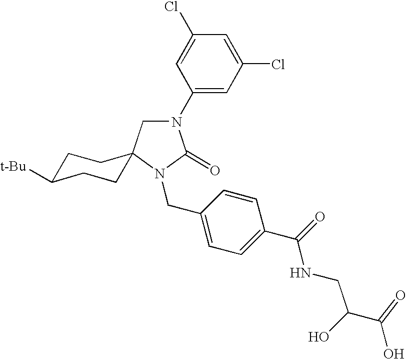 | 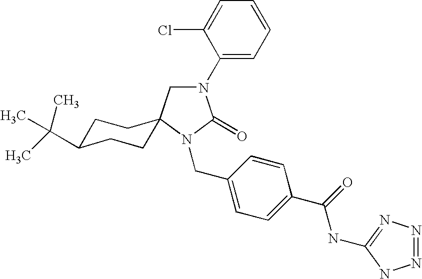 |
| 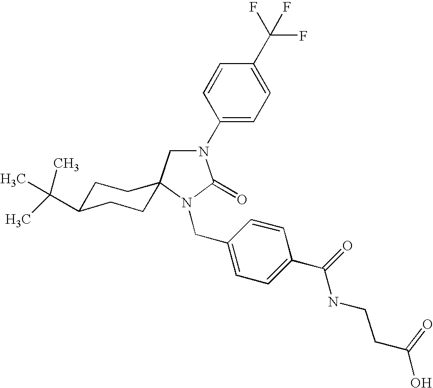 | 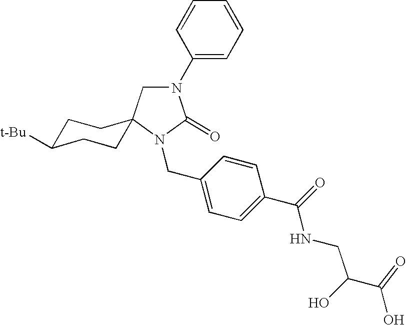 |
| 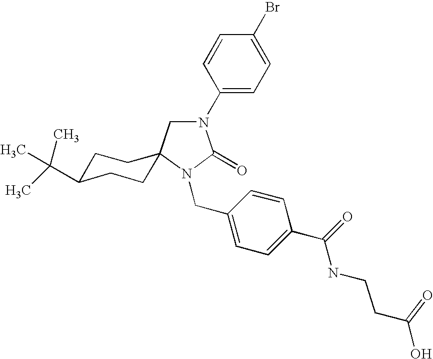 | 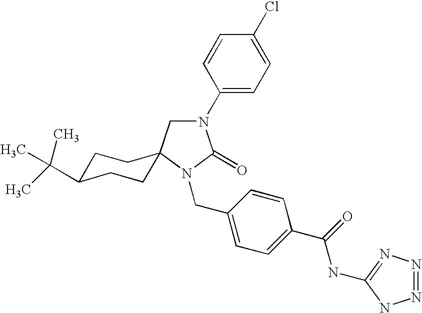 |
| 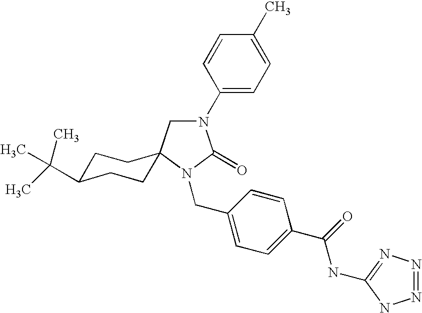 | 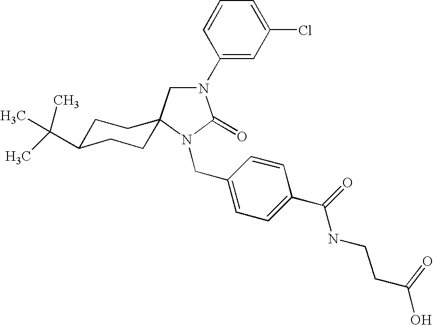 |
| 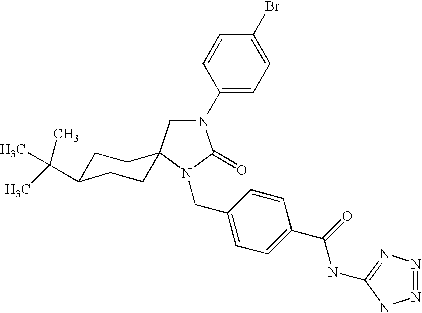 | 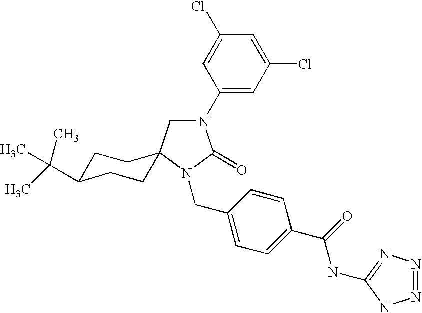 |
| 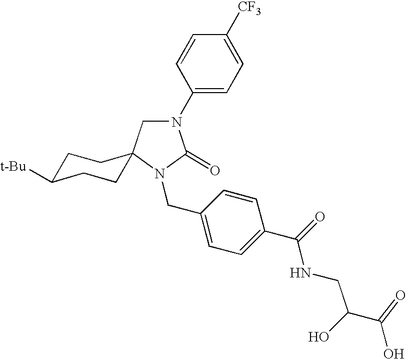 | 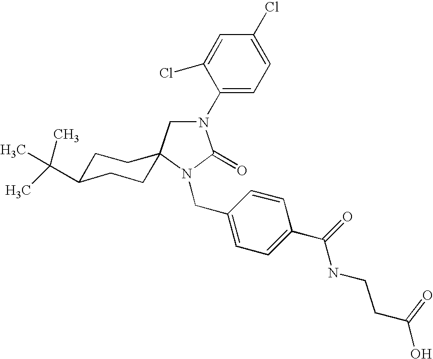 |
| 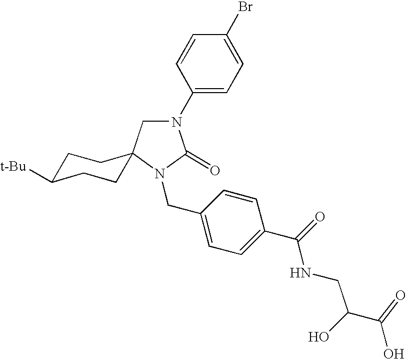 | 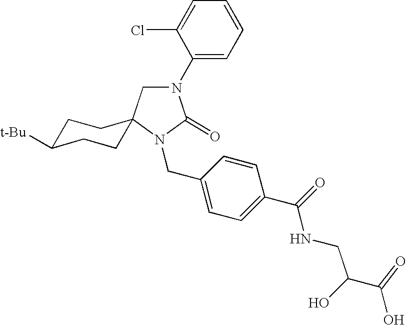 |
| 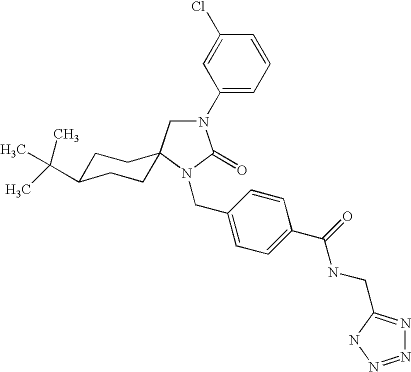 | 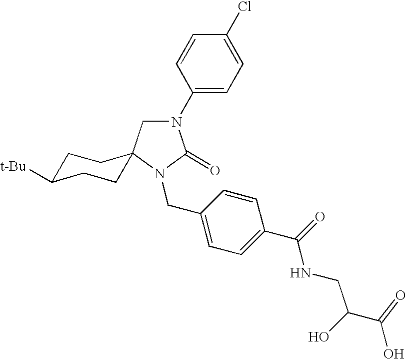 |
| 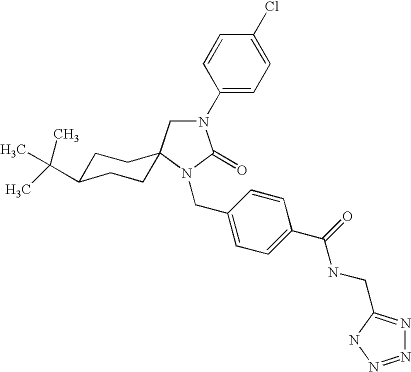 | 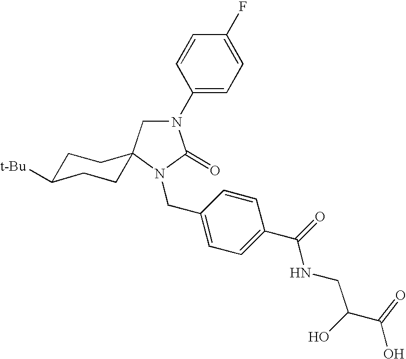 |
| 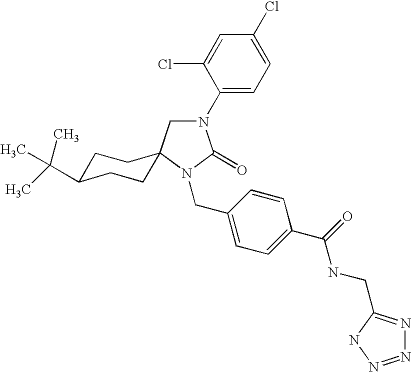 | 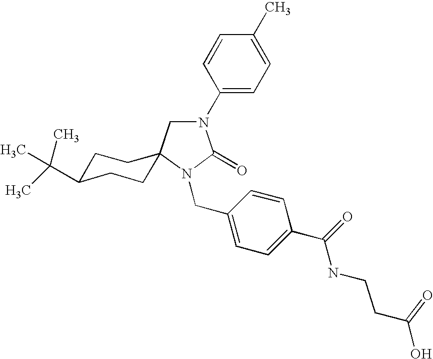 |
| 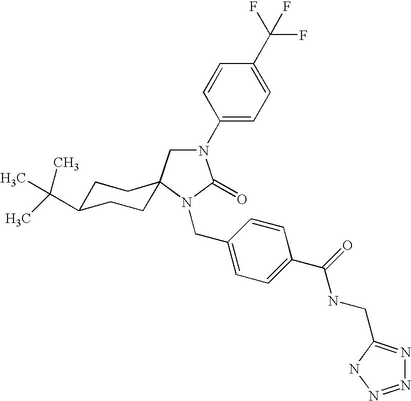 | 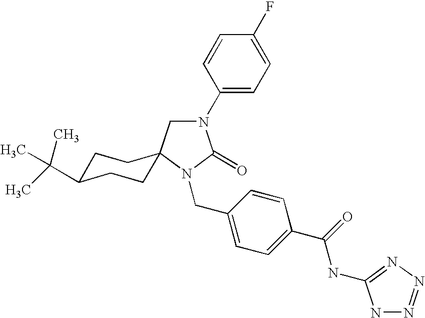 |
| 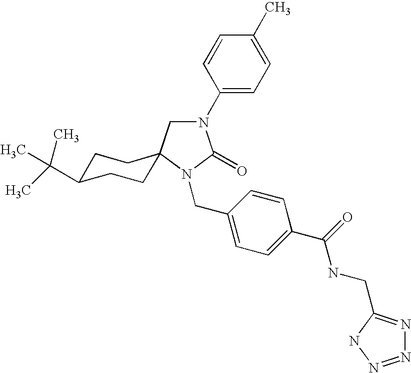 | 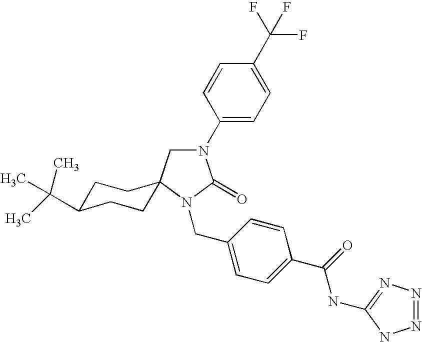 |
| 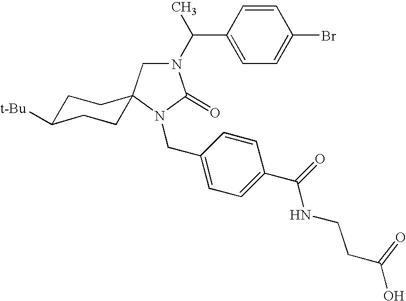 | 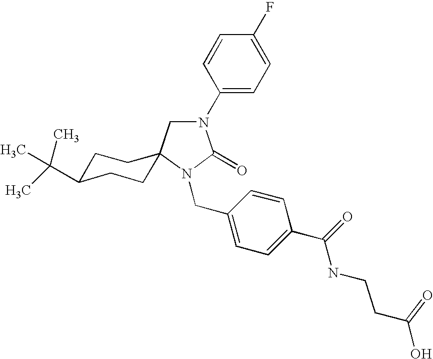 |
| 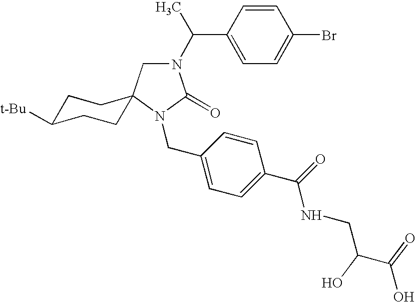 | 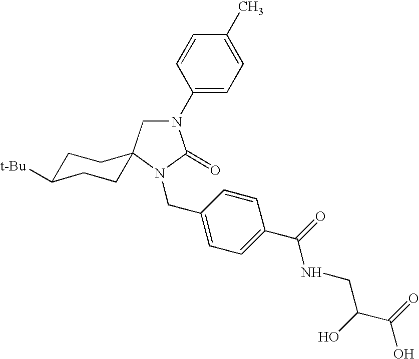 |
| 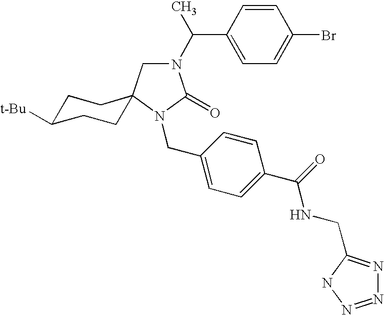 | 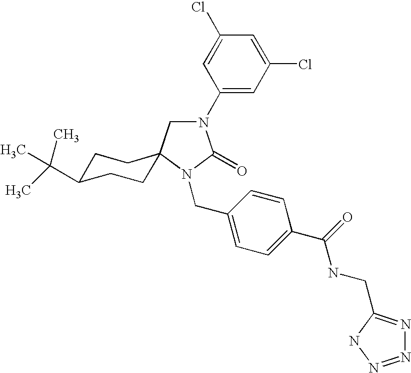 |
| 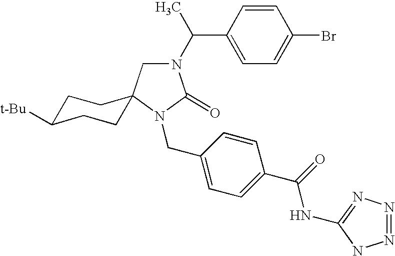 | 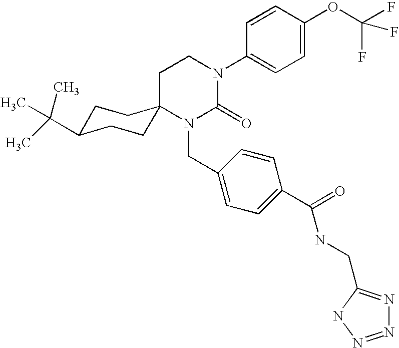 |
| 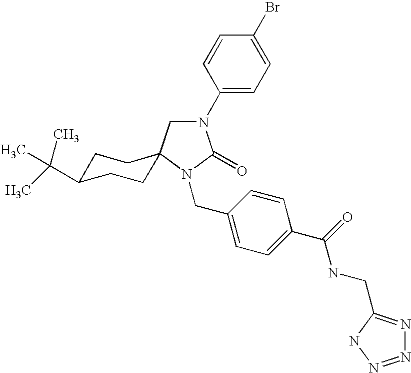 | 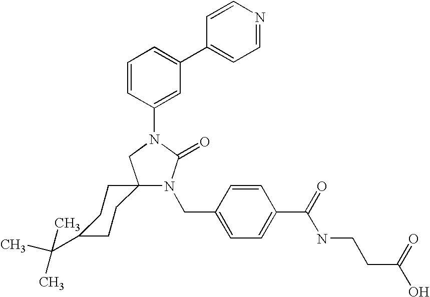 |
| 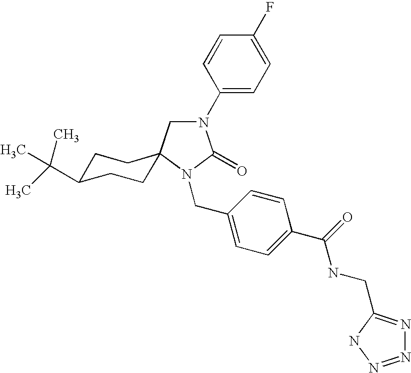 | 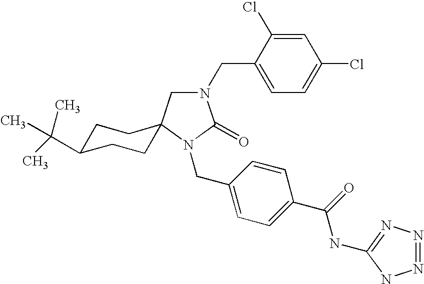 |
| 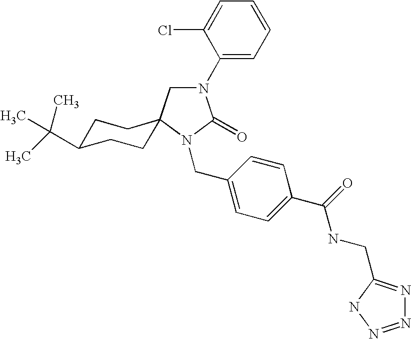 | 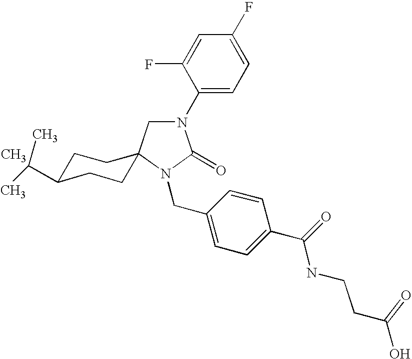 |
| 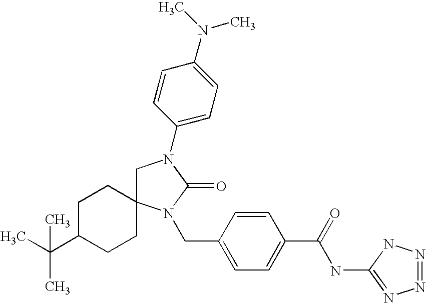 | 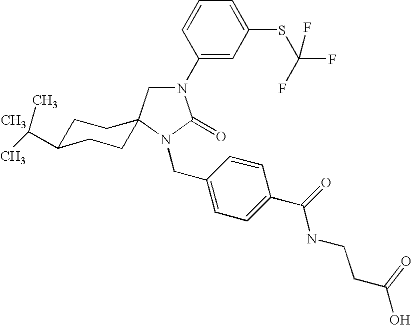 |
| 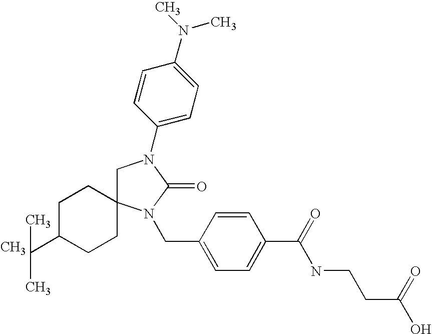 | 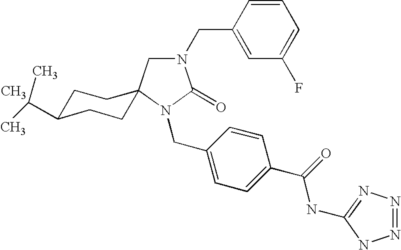 |
| 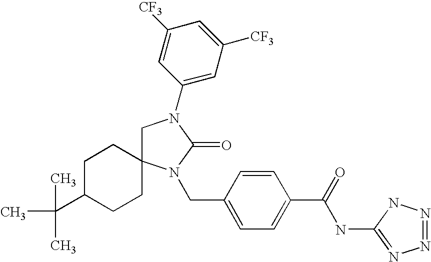 | 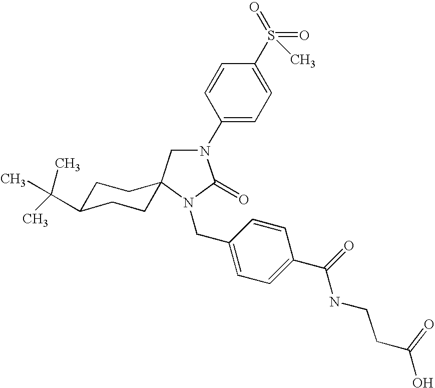 |
| 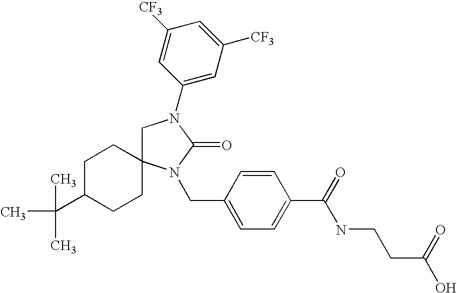 | 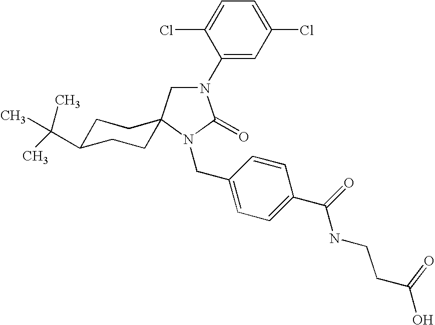 |
| 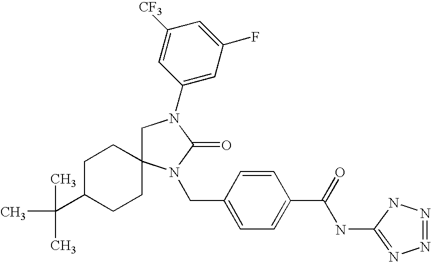 | 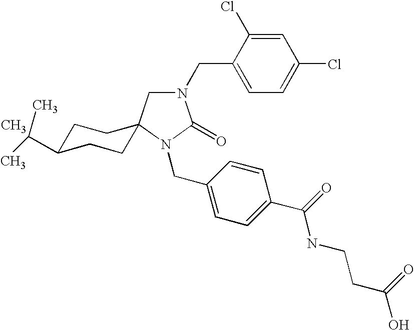 |
| 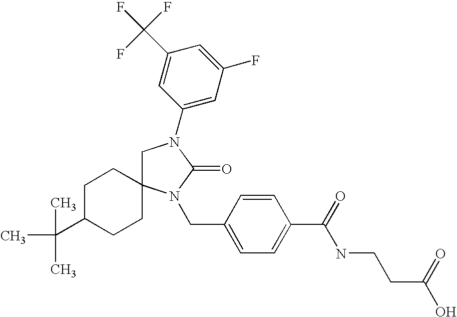 | 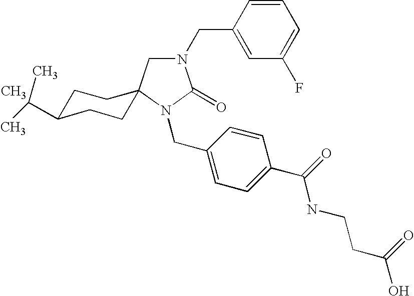 |
| 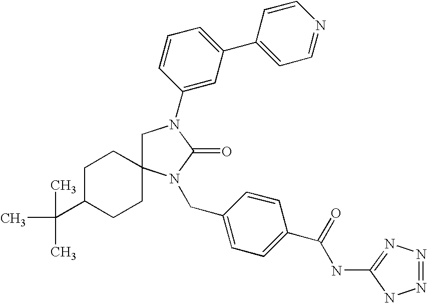 | 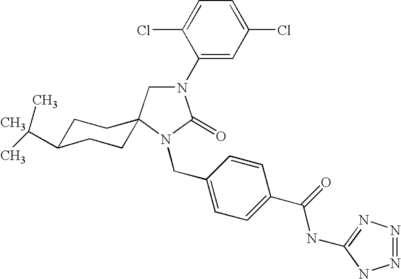 |
| 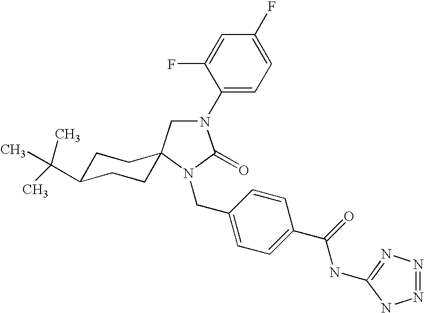 | 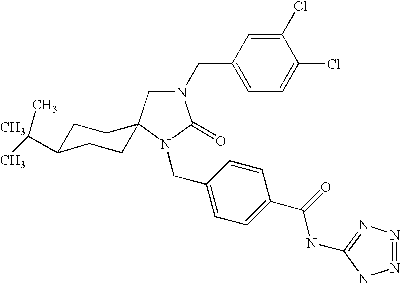 |
| 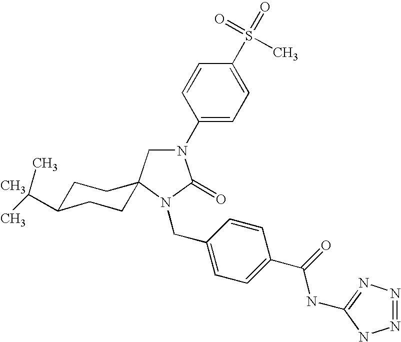 | 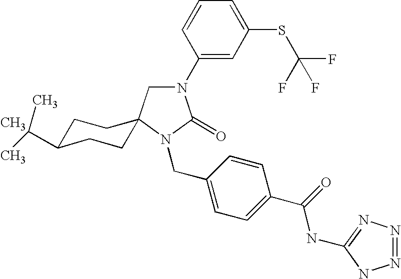 |
| 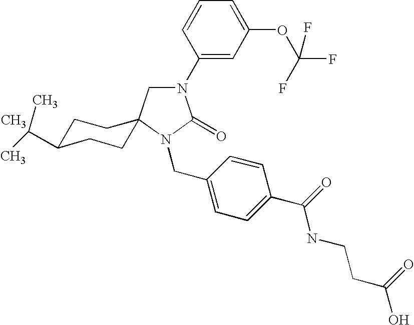 | 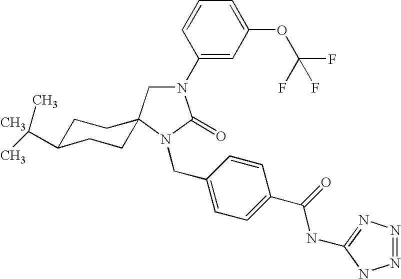 |
| 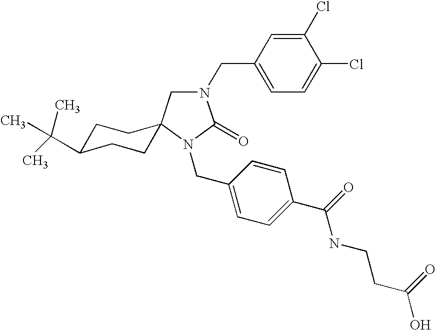 | 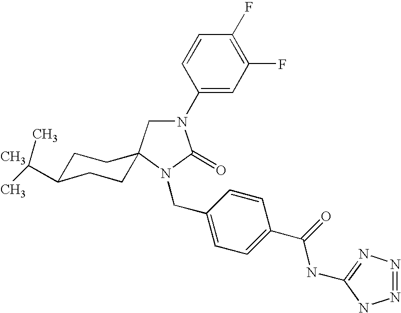 |
| 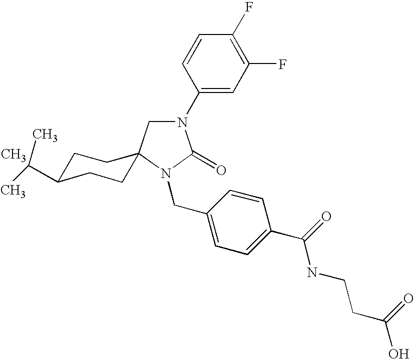 | 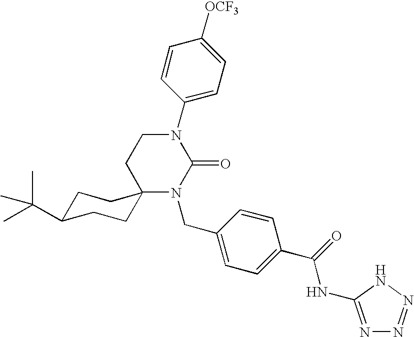 |
| 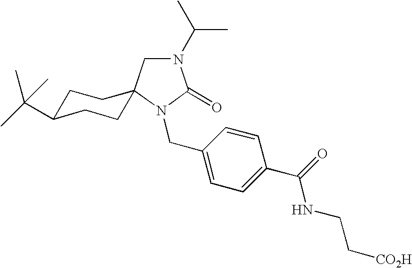 | |

or a pharmaceutically acceptable salt or solvate thereof. In many of the structures above, the hydrogen on one or more nitrogen atoms is implied.

The invention further includes a pharmaceutical composition which is comprised of a compound of formula I in combination with a pharmaceutically acceptable carrier.

Also included is a method of treating type 2 diabetes mellitus in a mammalian patient in need of such treatement, comprising administering to said patient a compound of formula I in an amount that is effective to treat type 2 diabetes mellitus.

Also included is a method of preventing or delaying the onset of type 2 diabetes mellitus in a mammalian patient in need thereof, comprising administering to said patient a compound of formula I in an amount that is effective to prevent or delay the onset of type 2 diabetes mellitus.

Also included is a method of treating, preventing or delaying the onset of diseases or conditions that are associated with type 2 diabetes mellitus. Examples include diseases and conditions selected from the group consisting of: dyslipidemias, such as elevated levels of cholesterol, triglycerides or low density lipoproteins (LDL), low levels of high density lipoprotein (HDL), microvascular or macrovascular changes and the sequellae of such conditions, such as coronary heart disease, stroke, peripheral vascular disease, hypertension, renal hypertension, nephropathy, neuropathy and retinopathy. The method entails administering to a type 2 diabetic patient, e.g., a human patient, an amount of a compound of formula I that is effective for treating, preventing or delaying the onset of such diseases or conditions.

Also included is a method of treating hyperglycemia, diabetes or insulin resistance in a mammalian patient in need of such treatment which comprises administering to said patient an effective amount of a compound in accordance with formula I.

Also included is a method of treating non-insulin dependent diabetes mellitus in a mammalian patient in need of such treatment comprising administering to the patient an anti-diabetic effective amount of a compound in accordance with formula I.

Also included is a method of treating obesity in a mammalian patient in need of such treatment comprising administering to said patient a compound in accordance with formula I in an amount that is effective to treat obesity.

Also included is a method of treating Syndrome X in a mammalian patient in need of such treatment, comprising administering to said patient a compound in accordance with formula I in an amount that is effective to treat Syndrome X.

Also included is a method of treating a lipid disorder selected from the group consisting of dyslipidemia, hyperlipidemia, hypertriglyceridemia, hypercholesterolemia, low HDL and high LDL in a mammalian patient in need of such treatment, comprising administering to said patient a compound in accordance with formula I in an amount that is effective to treat said lipid disorder.

Also included is a method of treating atherosclerosis in a mammalian patient in need of such treatment, comprising administering to said patient a compound in accordance with formula I in an amount effective to treat atherosclerosis.

Also included is a method of treating a condition selected from the group consisting of: (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, and other conditions and disorders where insulin resistance is a component, in a mammalian patient in need of such treatment, comprising administering to the patient a compound in accordance with formula I in an amount that is effective to treat said condition.

Also included is a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, and other conditions and disorders where insulin resistance is a component in a mammalian patient in need of such treatment, comprising administering to the patient a compound in accordance with formula I in an amount that is effective to delay the onset of said condition.

Also included is a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, and other conditions and disorders where insulin resistance is a component in a mammalian patient in need of such treatment, comprising administering to the patient a compound in accordance with formula I in an amount that is effective to reduce the risk of developing said condition.

Also included is a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) Syndrome X, and other conditions and disorders where insulin resistance is a component, in a mammalian patient in need of such treatment, comprising administering to the patient an effective amount of a compound in accordance with formula I, and a compound selected from the group consisting of: (a) DP-IV inhibitors; (b) insulin sensitizers selected from the group consisting of (i) PPAR agonists and (ii) biguanides; (c) insulin and insulin mimetics; (d) sulfonylureas and other insulin secretagogues; (e) α-glucosidase inhibitors; (f) glucagon receptor antagonists; (g) GLP-1, GLP-1 mimetics, and GLP-1 receptor agonists; (h) GIP,GIP mimetics, and GIP receptor agonists; (i) PACAP, PACAP mimetics, and PACAP receptor 3 agonists; (j) cholesterol lowering agents selected from the group consisting of (i) HMG-CoA reductase inhibitors, (ii) sequestrants, (iii) nicotinyl alcohol, nicotinic acid and salts thereof, (iv) PPARα agonists, (v) PPARα/γ dual agonists, (vi) inhibitors of cholesterol absorption, (vii) acyl CoA:cholesterol acyltransferase inhibitors, (viii) anti-oxidants and (ix) LXR modulators; (k) PPARδ agonists; (l) antiobesity compounds; (m) an ileal bile acid transporter inhibitor; (n) anti-inflammatory agents excluding glucocorticoids; and (o) protein tyrosine phosphatase-1B (PIP-1B) inhibitors, said compounds being administered to the patient in an amount that is effective to treat said condition.

Also included is a method of treating a condition selected from the group consisting of hypercholesterolemia, atherosclerosis, low HDL levels, high LDL levels, hyperlipidemia, hypertriglyceridemia and dyslipidemia, in a mammalian patient in need of such treatment, comprising administering to the patient a therapeutically effective amount of a compound in accordance with formula I and an HMG-CoA reductase inhibitor.

More particularly, the present invention includes the above method wherein the HMG-CoA reductase inhibitor is a statin.

Even more particularly, the present invention includes a method wherein the statin is selected from the group consisting of lovastatin, simvastatin, pravastatin, fluvastatin, atorvastatin, itavastatin, ZD-4522 and rivastatin.

Also included is a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions comprising administering to a mammalian patient in need of such treatment a therapeutically effective amount of a compound in accordance with formula I and an HMG-CoA reductase inhibitor.

Also included is a method for delaying the onset or reducing the risk of developing atherosclerosis in a human patient in need of such treatment comprising administering to said patient an effective amount of a compound in accordance with formula I, and an HMG-CoA reductase inhibitor.

In particular, a method is disclosed wherein the HMG-CoA reductase inhibitor is a statin.

Even more particularly, a method is disclosed wherein the statin is selected from the group consisting of: lovastatin, simvastatin, pravastatin, fluvastatin, atorvastatin, itavastatin, ZD-4522 and rivastatin.

Even more particularly, a method is disclosed wherein the statin is simvastatin.

Further, a method is disclosed wherein the invention further comprises administering a cholesterol absorption inhibitor.

More particularly, a method is disclosed wherein the cholesterol absorption inhibitor is ezetimibe.

More particularly, a method for delaying the onset or reducing the risk of developing atherosclerosis in a human patient in need of such treatment is disclosed comprising administering to said patient an effective amount of a compound in accordance with formula I and a cholesterol absorption inhibitor.

More particularly, the cholesterol absorption inhibitor is ezetimibe.

Another aspect of the invention relates to a pharmaceutical composition which is comprised of (1) a compound in accordance with formula I, (2) a compound selected from the group consisting of: (a) DP-IV inhibitors; (b) insulin sensitizers selected from the group consisting of (i) PPAR agonists and (ii) biguanides; (c) insulin and insulin mimetics; (d) sulfonylureas and other insulin secretagogues; (e) α-glucosidase inhibitors; (f) glucagon receptor antagonists; (g) GLP-1, GLP-1 mimetics, and GLP-1 receptor agonists; (h) GIP, GIP mimetics, and GIP receptor agonists; (i) PACAP, PACAP mimetics, and PACAP receptor 3 agonists; (j) cholesterol lowering agents selected from the group consisting of (i) HMG-CoA reductase inhibitors, (ii) sequestrants, (iii) nicotinyl alcohol, nicotinic acid or a salt thereof, (iv) PPARα agonists, (v) PPARα/γ dual agonists, (vi) inhibitors of cholesterol absorption, (vii) acyl CoA:cholesterol acyltransferase inhibitors, (viii) anti-oxidants and (ix) LXR modulators; (k) PPARδ agonists; (l) antiobesity compounds; (m) an ileal bile acid transporter inhibitor; (n) anti-inflammatory agents other than glucocorticoids; and (o) protein tyrosine phosphatase-1B (PIP-1B) inhibitors;

in combination with (3) a pharmaceutically acceptable carrier.

Many of the compounds of formula I contain one or more asymmetric centers and thus occur as racemates and racemic mixtures, single enantiomers, diastereomeric mixtures and individual diastereomers. The present invention includes all such isomeric forms of the compounds, in pure form as well as in mixtures.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

The term “pharmaceutically acceptable salts” refers to salts prepared from pharmaceutically acceptable substantially non-toxic bases or acids including inorganic or organic bases and inorganic or organic acids, as well as salts that can be converted into pharmaceutically acceptable salts. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

Solvates as used herein refers to the compound of formula I or a salt thereof, in association with a solvent, such as water. Representative examples include hydrates, hemihydrates, trihydrates and the like.

References to the compounds of Formula I include the pharmaceutically acceptable salts and solvates.

This invention relates to method of antagonizing or inhibiting the production or activity of glucagon, thereby reducing the rate of gluconeogenesis and glycogenolysis, and the concentration of glucose in plasma.

The compounds of formula I can be used in the manufacture of a medicament for the prophylactic or therapeutic treatment of disease states in mammals caused by elevated levels of glucose.

The prophylactic or therapeutic dose of a compound of formula I will, of course, vary with the nature of the condition to be treated, the particular compound selected and its route of administration. It will also vary according to the age, weight and response of the individual patient. In general, the daily dose range lie within the range of from about 0.001 mg to about 100 mg per kg body weight, preferably about 0.01 mg to about 50 mg per kg, and more preferably 0.1 to 10 mg per kg, in single or divided doses. It may be necessary to use dosages outside of these limits in some cases. The terms “effective amount” “anti-diabetic effective amount” and the other terms appearing throughout the application addressing the amount of the compound to be used refer to the dosage ranges provided, taking into account any necessary variation outside of these ranges, as determined by the skilled physician. Administration as used herein also includes the administration of a prodrug of a compound of formula I.

Representative dosages for adults range from about 0.1 mg to about 1.0 g per day, in single or divided doses.

When intravenous or oral administration is employed, a representative dosage range is from about 0.001 mg to about 100 mg (preferably from 0.01 mg to about 10 mg) of a compound of Formula I per kg of body weight per day, and more preferably, about 0.1 mg to about 10 mg of a compound of Formula I per kg of body weight per day.

As mentioned above, the pharmaceutical composition comprises a compound of Formula I or a pharmaceutically acceptable salt or solvate thereof and a pharmaceutically acceptable carrier. The term “composition” encompasses a product comprising the active and inert ingredient(s), (pharmaceutically acceptable excipients) that make up the carrier, as well as any product which results, directly or indirectly, from the combination, complexation or aggregation of any two or more of the ingredients, or from dissociation of one or more of the ingredients, or from other types of reactions or interactions between ingredients. Preferably the composition is comprised of a compound of formula I in an amount that is effective to treat, prevent or delay the onset of type 2 diabetes mellitus, in combination with the pharmaceutically acceptable carrier.

Any suitable route of administration may be employed for providing a mammal, especially a human with an effective dosage of a compound of the present invention. For example, oral, rectal, topical, parenteral, ocular, pulmonary, nasal, and the like may be employed. Examples of dosage forms include tablets, troches, dispersions, suspensions, solutions, capsules, creams, ointments, aerosols and the like, with oral tablets being preferred.

Thus, one aspect of the invention that is of interest is the use of a compound of formula I for preparing a pharmaceutical composition which is comprised of combining the compound of formula I with the carrier. In preparing oral compositions, any of the usual pharmaceutical media may be employed, such as, for example, water, glycols, oils, alcohols, flavoring agents, preservatives, coloring agents and the like in the case of oral liquids, e.g., suspensions, elixirs and solutions; or carriers such as starches, sugars, microcrystalline cellulose, diluents, granulating agents, lubricants, binders, disintegrating agents and the like in the case of oral solids, e.g., powders, capsules and tablets, with the solid oral preparations being preferred. Because of their ease of administration, tablets and capsules represent the most advantageous oral dosage unit forms. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 1 g of the active ingredient and each cachet or capsule contains from about 1 to about 500 mg of the active ingredient.

The following are examples of pharmaceutical dosage forms for the compounds of Formula I:

| Injectable Suspension (I.M.) | mg/mL | Tablet | mg/tablet |
|---|---|---|---|
| Compound of Formula I | 10 | Compound of Formula I | 25 |
| Methylcellulose | 5.0 | Microcrystalline Cellulose | 415 |
| Tween 80 | 0.5 | Povidone | 14.0 |
| Benzyl alcohol | 9.0 | Pregelatinized Starch | 43.5 |
| Benzalkonium chloride | 1.0 | Magnesium Stearate | 2.5 |
| Water for injection to make | 1.0 mL | Total | 500 mg |
| Capsule | mg/capsule | Aerosol | Per canister |
| Compound of Formula I | 25 | Compound of Formula I | 24 mg |
| Lactose Powder | 573.5 | Lecithin, NF Liq. Conc. | 1.2 mg |
| Magnesium Stearate | 1.5 | Trichlorofluoromethane, NF | 4.025 g |
| Total | 600 mg | Dichlorodifluoromethane, NF | 12.15 g |

Compounds of Formula I may be used in combination with other drugs that are used in the treatment/prevention/delaying the onset of type 2 diabetes mellitus, as well as the diseases and conditions associated with type 2 diabetes mellitus, for which compounds of Formula I are useful. Other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (a) bis-guanides (e.g., buformin, metformin, phenformin), (b) PPAR agonists (e.g., troglitazone, pioglitazone, rosiglitazone), (c) insulin, (d) somatostatin, (e) α-glucosidase inhibitors (e.g., voglibose, miglitol, acarbose), (f) DP-IV inhibitors, (g) LXR modulators and (h) insulin secretagogues (e.g., acetohexamide, carbutamide, chlorpropamide, glibornuride, gliclazide, glimerpiride, glipizide, gliquidine, glisoxepid, glyburide, glyhexamide, glypinamide, phenbutamide, tolazamide, tolbutamide, tolcyclamide, nateglinide and repaglinide).

The weight ratio of the compound of the Formula I to the second active ingredient may be varied within wide limits and depends upon the effective dose of each ingredient. Generally, an effective dose of each will be used. Thus, for example, when a compound of the Formula I is combined with a PPAR agonist the weight ratio of the compound of the Formula I to the PPAR agonist will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

Throughout the instant application, the following abbreviations are used with the following meanings unless otherwise indicated:

| Bu = butyl | Bn = benzyl | eq. = equivalent(s) |
|---|---|---|
| BOC, Boc = t-butyloxycarbonyl | | CBZ, Cbz = Benzyloxycarbonyl |
| DCC = Dicyclohexylcarbodiimide | | DCM = dichioromethane |
| DIEA = diisopropylethylamine | | DMF = N,N-dimethylformamide |
| DMAP = 4-Dimethylaminopyridine | | THF = Tetrahydrofuran |
| EtOAc = ethyl acetate | | EtOH = ethanol |
| HOAc = acetic acid | Me = methyl | FAB-mass spectrum = Fast atom bombardment- |
| Ph = phenyl | Et = ethyl | mass spectroscopy |
| 2CIPh = 2-chlorophenyl | | HPLC = High pressure liquid chromatography |
| HOBT, HOBt = Hydroxybenztriazole | | LAH = Lithium aluminum hydride |
| 2,4-diCIPh = 2,4-dichlorophenyl | | PBS = phosphate buffer saline |
| iPr = isopropyl | C₆H₁₁ = cyclohexyl | TFA = Trifluoroacetic acid |

| | |
|---|---|
| NMe₂ = dimethylamino | TMS = Trimethylsilane |

Compounds of the present invention may be prepared according to the methodology outlined in the following general synthetic schemes.

In one embodiment of the present invention, the compounds (Ia) may be prepared by alkylation of cyclic urea IIa: 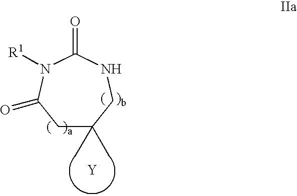
where X from formula I represents a carbonyl group and R¹, Y, a, and b are as defined with respect to formula I.

Many of the intermediates described herein, e.g., compounds of formula IIa and IIb, are generally known in the literature or may be conveniently prepared by a variety of methods familiar to those skilled in the art, such as described in Katritsky et al., Advances in Heterocyclic Chemistry, Vol. 38, 1985, pg 177 and references therein. One such route when a and b are both zero is illustrated below in Scheme 1.

Ketone 1, which may be commercially available or readily prepared from the corresponding alcohol, is subjected to Bucherer-Bergs reaction conditions, i.e. aqueous potassium cyanide and ammonium chloride in a polar solvent such as methanol, to give the corresponding amino nitrile 2 (Edward et. al., Can. J. Chem., 53, 3339 (1975). This is then converted to the cyclic compound 3 by addition of an isocyanate 4 in a solvent such as benzene for 1 to 24 h at ambient temperature, followed by the addition of a base, such as sodium hydride to effect cyclization. Intermediates 4 are commercially available or readily prepared from the corresponding amine by reaction with phosgene or an equivalent reagent and a base, for example triethylamine, in a solvent such as dichloromethane or toluene at 0° C. for 1 to 16 h. Intermediate IIa-1, wherein a and b are 0, is then prepared by hydrolysis of the imino group under acidic conditions, for example using dilute hydrochloric acid in an alcoholic solvent such as ethanol at temperatures of 70 to 100° C. for 1 to 6 h. As will be understood by those skilled in the art, for the preparation of enantiomerically pure compounds, enantiomerically pure starting materials should be used.

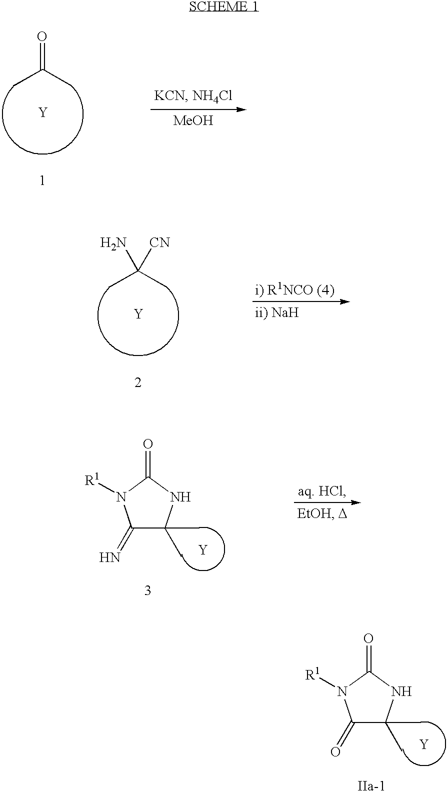

An alternate route to the amino nitrile 5 wherein the resulting intermediate in which a represents 1, is shown in Scheme 2 (see Kumamoto et. al., Chem. Pharm. Bull., 45, 753-755, (1997) and Suzuki et. al., Synth Commun., 28, 701-712, (1998) for related work). Ketone 1 is condensed with the anion of diethyl (cyanomethyl)phosphonate, formed by treatment of the phosphonate with a base such as butyl lithium in a solvent, normally tetrahydrofuran (THF) or toluene. The reaction is stirred at −78° C. for 1 to 6 h to give the unsaturated compound 6. Treatment with ammonia in aqueous methanol in a sealed system at elevated temperatures of 100 to 150° C. for 16 to 48 h yields the amino nitrile 5. In some cases mixtures of isomers will be formed. These are generally separable by recrystallization, trituration, preparative thin layer chromatography, flash chromatography on silica gel as described by W. C. Still et al, J. Org. Chem., 43, 2923, (1978), or HPLC. Compounds purified by HPLC may be isolated as the corresponding salt. The cyclized product is then prepared from 5 as described above.

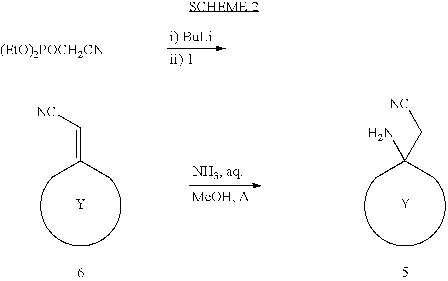

Another embodiment of the invention involves the preparation of intermediates IIb: 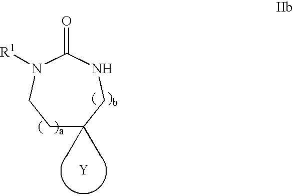
wherein X from formula I represents a CH₂ group, and R¹, Y, a, and b, are as defined with respect to formula I.

Synthesis of compounds of formula IIb may involve the reduction of the dicarbonyl intermediate IIa. This, as described in Knabe et. al., Arch. Pharm., 326, p 79, (1993), can be achieved using, e.g., a hydride reducing agent such as lithium aluminium hydride in the presence of a Lewis acid, e.g., aluminium trichloride in a polar aprotic solvent, such as THF, at a temperature from about 0 to about 25° C. followed by an aqueous work up.

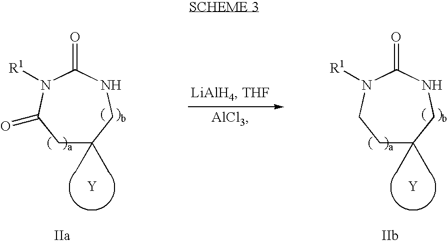

An alternate route to intermediates IIb-1, where a is equal to 0, b is equal to one and X represents CH₂, involves alkylation of a primary urea 7, as disclosed in Scheme 4. Compounds 7 can be readily prepared from the corresponding amine, aqueous sodium cyanate, and an acid, such as acetic acid, as described in J. Chem. Soc., p 1031, (1946). This is then converted to the cyclic urea by coupling to a dibromo or bissulfonate derivative 8. The reaction is generally performed under biphasic conditions, using a solvent such as benzene and an aqueous base, normally sodium hydroxide, in the presence of a phase transfer catalyst such as benzyl triethylammonium bromide at temperatures of 25 to 80° C. for up to 4 days. This reaction is further described in Cram et. al., J. Am. Chem. Soc., 106, p 4987, (1984), and Vol. 112, p 5837, (1990).

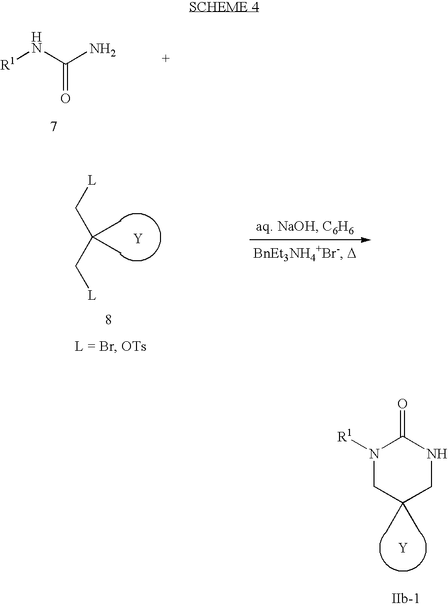

Intermediate II can then be converted to compounds I-10 and I-11 as shown in Scheme 5. Alkylation of cyclic urea II with, for example, 4-carbomethoxybenzylbromide can be achieved following deprotonation of the urea with a base such as sodium hydride or cesium carbonate in a polar solvent, generally dimethyl formamide (DMF), at 0 to 25° C. for 3 to 24 h. Saponification of the methyl ester 9 is then achieved using a base such as aqueous lithium or sodium hydroxide in a polar solvent such as tetrahydrofuran, methanol, ethanol or a mixture of similar solvents. Coupling of the acid with an amine, generally 5-aminotetrazole 10 or a beta alanine derivative 11 which may be substituted at the 2-position, is then achieved using, e.g., 1-ethyl-3-(3-dimethylaminopropyl)-carbodiimide (EDC), 1-hydroxybenzotriazole (HOBt), and a base, generally diisopropylethylamine, in a solvent such as N,N-dimethylformamide (DMF) or methylene chloride for 3 to 48 hours at ambient temperature to yield a compound of formula I, such as the tetrazole I-10 or the carboxylic acid I-11. The product is purified from unwanted side products by recrystallization, trituration, preparative thin layer chromatography, flash chromatography on silica gel as described by W. C. Still et al, J. Org. Chem., 43, 2923, (1978), or HPLC. Purification of intermediates is achieved in the same manner.

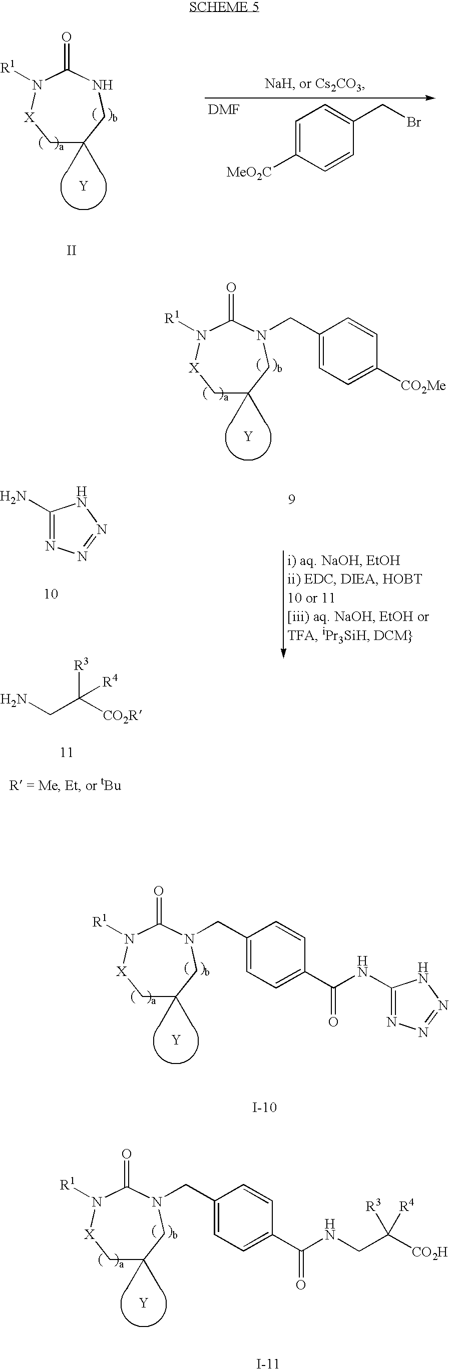

In some cases, the product from the reactions described in Schemes 1-5 is further modified. These manipulations may include, but are not limited to substitution, reduction, oxidation, alkylation, acylation and hydrolysis reactions, which are commonly known to those skilled in the art. One such modification is removal of an ester, as shown. For a methyl ester this is achieved using a base such as aqueous lithium or sodium hydroxide in a polar solvent such as tetrahydrofuran, methanol, ethanol or a mixture of similar solvents, while a tert-butyl ester is generally cleaved using trifluoroactetic acid and triisopropylsilane in dichloromethane or similar solvent.

An alternate route to the products I-10 and I-11 involves cyclization of a hydroxy urea as described in Rapaport et. al., J. Org. Chem., 55, p 3699, (1990) and shown in Scheme 6. Protected amino alcohols are commercially available or may be conveniently prepared by a variety of methods familiar to those skilled in the art. One such method involves reduction of an amino acid by formation of the mixed anhydride, usually with carbonyldiimidazole or isobutyl chloroformate and a base such as triethylamine in a solvent such as THF, followed by addition of a reducing agent, normally aqueous sodium borohydride at 0 to 25° C. Alternatively, amino alcohol 12 can be prepared by direct reduction of the acid using borane.THF complex in a solvent such as THF at 25 to 60° C. for 16 to 24 h.

Removal of the nitrogen protecting group, using trifluoroacetic acid in the case of a tert-butyl protecting group, is then followed by alkylation of the nitrogen. This is normally achieved via a reductive amination sequence using, for example, 4-carbomethoxy benzaldehyde 13 and a reducing agent such as sodium triacetoxycyanoborohydride in a solvent such as dichloroethane at ambient temperatures. Compound 14 can also be prepared from diol 15, by oxidation to the keto alcohol, most conveniently with one equivalent of Dess-Martin reagent (J. Am. Chem. Soc., 113, p 7277, (1991)). This followed by reductive amination using 4-carbomethoxybenzyl-amine and a reducing agent such as sodium triacetoxycyanoborohydride in a solvent such as dichloroethane at ambient temperatures.

Addition of an isocyanate 4 in a solvent such as methylene chloride for 1 to 24 h at ambient temperature, gives the cyclization precursor 16. This is converted to the cyclic urea 17 by treatment with triphenyl phosphine and diethylazodicarboxylate in a polar aprotic solvent such as THF at ambient temperature for 1 to 3 h. In some cases the isomeric oxazolidinone imine will also be formed in this reaction. The compounds are generally separable by preparative thin layer chromatography, flash chromatography on silica gel as described by W. C. Still et al, J. Org. Chem., 43, 2923, (1978), or HPLC. Saponification of the methyl ester 17 is then achieved using a base such as aqueous lithium or sodium hydroxide in a polar solvent such as tetrahydrofuran, methanol, ethanol or a mixture of similar solvents. Coupling of the acid with an amine, generally 5-aminotetrazole 10 or a beta alanine derivative 11 which may be substituted at the 2-position, is then achieved using 1-ethyl-3-(3-dimethylaminopropyl)-carbodiimide (EDC), 1-hydroxybenzotriazole (HOBt), and a base, generally diisopropylethylamine, in a solvent such as N,N-dimethylformamide (DMF) or methylene chloride for 3 to 48 hours at ambient temperature to yield a compound of formula I. The product is purified from unwanted side products by recrystallization, trituration, preparative thin layer chromatography, flash chromatography on silica gel as described by W. C. Still et al., J. Org. Chem., 43, 2923, (1978), or HPLC. Purification of intermediates is achieved in the same manner.

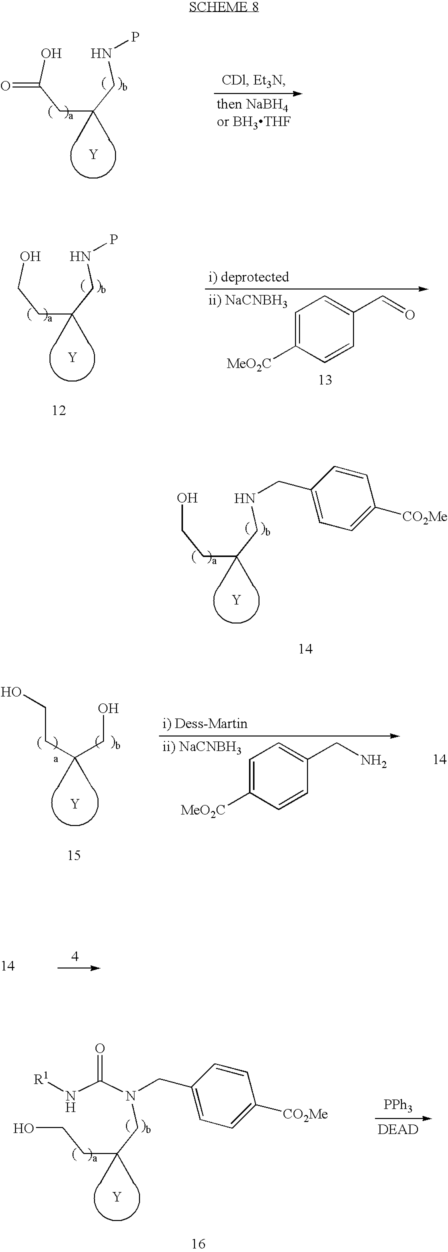

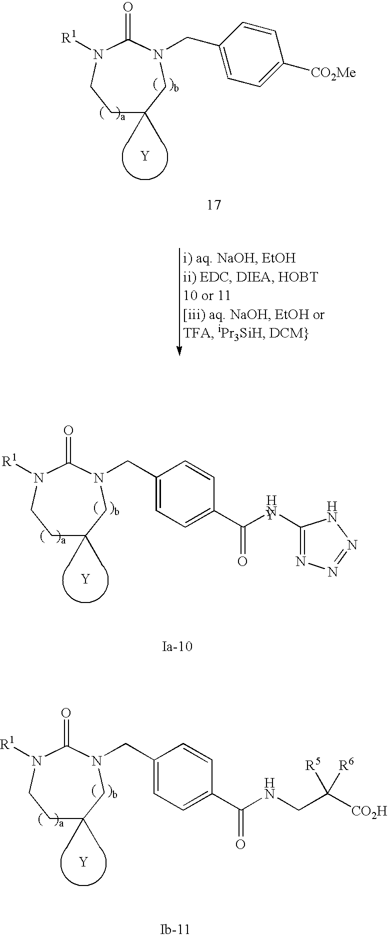

In some cases, the product from the reactions described in Scheme 6 is further modified. These manipulations may include, but are not limited to substitution, reduction, oxidation, alkylation, acylation, and hydrolysis reactions, which are commonly known to those skilled in the art. One such modification is removal of an ester, as shown. For a methyl ester this is achieved using a base such as aqueous lithium or sodium hydroxide in a polar solvent such as tetrahydrofuran, methanol, ethanol or a mixture of similar solvents, while a tert-butyl ester is generally cleaved using trifluoroactetic acid and triisopropylsilane in dichloromethane or similar solvent.

Protected amino acids may be commercially available or readily prepared from the corresponding amino acid by protection using, for example, N-(9-fluorenylmethoxycarbonyloxy)succinimide. In some cases where a beta amino acid is required, acids 18 are prepared by treatment of the alpha amino acid with isobutylchloroformate and diazomethane using a base such as triethylamine, Scheme 7. The resultant diazoketone is then treated with silver benzoate in aqueous dioxane and may be subjected to sonication following the procedure of Sewald et al., Synthesis, 837 (1997) in order to provide the beta amino acid 18. As will be understood by those skilled in the art, for the preparation of enantiomerically pure beta amino acids, enantiomerically pure alpha amino acids may be used. Alternate routes to these compounds can be found in the following reviews: E. Juaristi, Enantioselective Synthesis of β-Amino Acids, Ed., Wiley—VCH, New York: 1997, Juaristi et al., Aldrichimica Acta, 27, 3 (1994), Cole et al., Tetrahedron, 32, 9517 (1994).

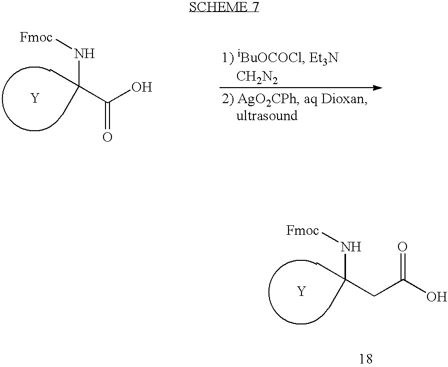

The following are non-limiting examples are provided to illustrate the invention.

### EXAMPLE 1

### 4-({TRANS-8-TERT-BUTYL-2,4-DIOXO-3-[4-(TRIFLUOROMETHOXY)PHENYL]-1,3-DIAZASPIRO[4.5]DEC-1-YL}METHYL)-N-(1H-TETRAZOL-5-YL)BENZAMIDE

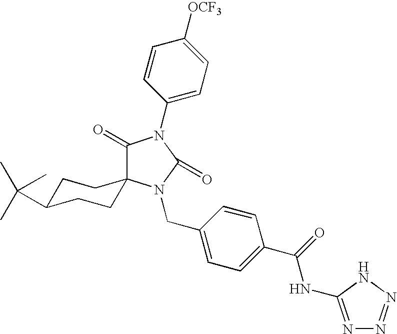

### Step A. Trans-1-amino-4-tert-butylcyclohexanecarbonitrile

After stirring a mixture of 30 g 4-t-butylcyclohexanone, 13 g potassium cyanide, 11 g ammonium chloride and 150 mL each of methanol and water for two days at room temperature, the resulting white precipitate was filtered and washed with water. This crude product was purified on silica gel column using 5 to 50% EtOAc in hexanes to give the title compound as a white solid. ¹H NMR (CDCl₃, 500 MHz) δ 2.06˜2.11 (m, 2H), 1.81˜1.87 (m, 4H), 1.32˜1.49 (m, 4H), 1.02 (tt, J=3 & 12 Hz, 1H), 0.90 (s, 9H).

### Step B. Trans-8-tert-butyl-4-imino-3-[4-(trifluoromethoxy)phenyl]-1,3-diazaspiro[4.5]-decan-2-one

A mixture of 27 g of the product from Step A above and 15.2 g 4-(trifluoromethoxy)phenyl isocyanate in 400 mL benzene was stirred at room temperature for 8 h, when LC-MS showed no more starting material. To this mixture was added 3 g 60% sodium hydride oil dispersion. After stirring 30 minutes at room temperature, the reaction mixture was poured into saturated ammonium chloride and extracted with ethyl acetate. The crude product from the organic phase was purified on silica gel with 2:1 hexanes and ethyl acetate to give the title compound as a white solid. LC-MS: 4.05 min. (M+H=384.2).

### Step C. Trans-8-tert-butyl-3-[4-(trifluoromethoxy)phenyl]-1,3-diazaspiro[4.5]decane-2,4-dione

A mixture of 15 g of the product from Step B above and 500 mL 1.5 M hydrochloric acid in 1 L ethanol was refluxed for 3 h. The organic solvent was removed under reduced pressure. The solid from the residue was filtered and washed with water, 1 N aq. NaOH, and dried to give the title compound as white solid. ¹H NMR (DMSO-d₆, 500 MHz) δ 8.41 (s, 1H), 7.48˜7.52 (m, 2H), 7.43˜7.47 (m, 2H), 2.06 (d, J=13.5 Hz, 2H), 1.52˜1.72 (m, 6H), 0.99 (tt, J=4 & 12 Hz, 1H), 0.84 (s, 9H). LC-MS: 2.31 min. (M+H=385.3).

### Step D. Tert-butyl 4-(bromomethyl)benzoate

N,N-Dimethylformamide di-tert-butylacetal (47.2 g) was added slowly to a refluxing suspension of 12.5 g 4-bromomethylbenzoic acid in 100 mL benzene. The reaction mixture was refluxed for additional 20 minutes after completing the addition. The reaction mixture was concentrated under vacuum and the resulting residue purified on silica gel with 2.5% ethyl acetate in hexanes to give the title compound as colorless oil. ¹H NMR (CDCl₃, 500 MHz) δ 7.96˜7.99 (m, 2H), 7.43˜7.46 (m, 2H), 4.51 (s, 2H), 1.61 (s, 9H).

### Step E. Tert-butyl 4-({trans-8-tert-butyl-2,4-dioxo-3-[4-(trifluoromethoxy)phenyl]-1,3-diazaspiro[4.5]dec-1-yl}methyl)benzoate

To a solution of 0.5 g product from Step C above in 10 mL dimethylformamide (DMF) was added 58 mg 60% sodium hydride oil dispersion. After stirring at room temperature for 15 minutes, 388 mg product from Step D was added. The resulting mixture was stirred at room temperature for 14 hours, poured into saturated ammonium chloride, and extracted with ethyl acetate. The crude product from the organic phase was purified on silica gel using 10 to 20% ethyl acetate in hexanes to give the title compound as a white solid. ¹H NMR (CDCl₃, 500 MHz) δ 7.97˜7.99 (m, 2H), 7.54˜7.57 (m, 2H), 7.41 (d, J=8.5 Hz, 2H), 7.31˜7.34 (m, 2H), 4.65 (s, 2H), 1.88˜1.92 (m, 2H), 1.79˜1.84 (m, 2H), 1.67˜1.73 (m, 4H), 1.61 (s, 9H), 10.97 (tt, J=3 & 12 Hz, 1H), 0.92 (s, 9H). LC-MS: 2.86 min. (M+H=575.3).

### Step F. 4-({Trans-8-tert-butyl-2,4-dioxo-3-[4-(trifluoromethoxy)phenyl]-1,3-diazaspiro[4.5]dec-1-yl}methyl)benzoic acid

To a solution of 0.5 g product from Step E above in 10.5 mL dichloromethane was added 4.5 mL trifluoroacetic acid. The reaction mixture was concentrated under vacuum after 20 minutes. The resulting residue was purified on silica gel using 2:1 ethyl acetate and hexanes to give the title compound as a pale solid.

### Step G. 4-({Trans-8-tert-butyl-2,4-dioxo-3-[4-(trifluoromethoxy)phenyl]-1,3-diazaspiro[4,5]dec-1-yl}methyl)-N-(1H-tetrazol-5-yl)benzamide

A solution of 100 mg product from Step F above, 56 mg 1-(3-dimethylaminopropyl)-3-ethylcarbodiimide hydrochloride (EDC), and 39.2 mg 1-hydroxybenzotriazole hydrate (HOBt) in 5 mL DMF was stirred at room temperature for 20 minutes. To this solution was added 26 mg 5-aminotetrazole monohydrate. The resulting mixture was stirred at room temperature for 8 hours. The reaction product was precipitated by adding 5 mL water and collected by centrifuge. This crude product was purified on reverse-phase HPLC to give the title compound as a white solid. LC-MS: 2.33 min. (M+H=586.2).

### EXAMPLE 2

### 4-({TRANS-8-TERT-BUTYL-2-OXO-3-[4-(TRIFLUOROMETHOXY)PHENYL]-1,3-DIAZASPIRO[4.5]DEC-1-YL}METHYL)-N-(1H-TETRAZOL-5-YL)BENZAMIDE

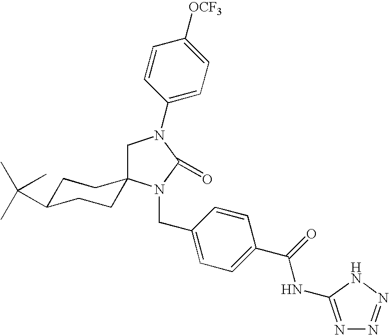

### Step A. Trans-8-tert-butyl-3-[4-(trifluoromethoxy)phenyl]-1,3-diazaspiro[4.5]decan-2-one

To a solution of 1 g product from Step C of Example 1 in 10 mL ether was added 2.6 mL 1 M lithium aluminum hydride (LAH) in ether at room temperature. After 3 hours, it was poured into saturated ammonium chloride and extracted with ether. The organic layer was washed with water and saturated brine. Evaporation under vacuum gave the title compound as a white solid. LC-MS: 2.44 min. (M+H=371.2).

### Step B. Trans-tert-butyl 4-({8-tert-butyl-2-oxo-3-[4-(trifluoromethoxy)phenyl]-1,3-diazaspiro[4.5]dec-1-yl}methyl)benzoate

Using the same procedure from Step E Example 1 and starting with the product from Step A above gave the title compound. ¹H NMR (CDCl₃, 500 MHz) δ 7.94 (d, J=8 Hz, 2H), 7.63˜7.67 (m, 2H), 7.40 (d, J=8 Hz, 2H), 7.22 (d. J=9 Hz, 2H), 4.50 (s, 2H), 3.63 (s, 2H), 1.79 (br d, J=13 Hz, 2H), 1.56˜1.65 (m, 4H), 1.06˜1.15 (m, 2H), 0.93˜0.99 (m, 1H), 0.87 (s, 9H). LC-MS: 2.92 min. (M+H=561.3).

### Step C. 4-({Trans-8-tert-butyl-2-oxo-3-[4-(trifluoromethoxy)phenyl]-1,3-diazaspiro[4.5]dec-1-yl}methyl)benzoic acid

Using the same procedure from Step F Example 1 and starting with the product from Step B above gave the title compound.

### Step D. 4-({Trans-8-tert-butyl-2-oxo-3-[4-(trifluoromethoxy)phenyl]-1,3-diazaspiro[4.5]dec-1-yl}methyl)-N-(1H-tetrazol-5-yl)benzamide

Using the same procedure from Step G Example 1 and starting with the product from Step C above gave the title compound. ¹H NMR (DMSO-d₆, 500 MHz) δ 7.95 (d, J=8 Hz, 2H), 7.76 (d, J=9 Hz, 2H), 7.45 (d, J=8 Hz, 2H), 7.31 (d, J=9 Hz, 2H), 4.46 (s, 2H), 3.73 (s, 2H), 1.59˜1.68 (m, 4H), 1.54 (br d, J=12.5 Hz, 2H), 1.11˜1.21 (m, 2H), 0.95˜1.00 (m, 1H), 0.82 (s, 9H). LC-MS: 4.29 min. (M+H=572.1).

### EXAMPLE 3

### ETHYL N-[4-({8-TERT-BUTYL-2-OXO-3-[4-(TRIFLUOROMETHOXY)PHENYL]-1,3-DIAZASPIRO[4.5]DEC-1-YL}METHYL)BENZOYL]-β-ALANINATE

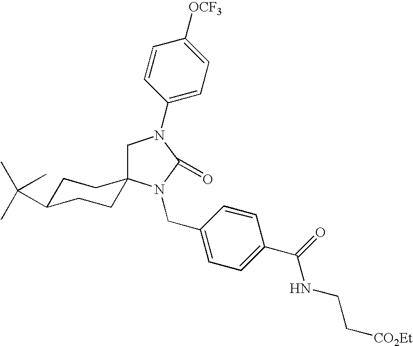

A solution of 100 mg product from Step C Example 2, 56 mg EDC, 66 μL diisopropylethylamine (DIEA) and 40 mg HOBt in 5 mL DMF was stirred at room temperature for 30 minutes. To this solution was added 38.4 mg β-alanine ethyl ester hydrochloride. The resulting mixture was stirred at room temperature for 8 hours. The reaction product was precipitated by adding 5 mL water and collected by centrifuge. This crude product was purified on reverse-phase HPLC to give the title compound as a white solid. ¹H NMR (CDCl₃, 500 MHz) δ 7.71 (d, J=8.5 Hz, 2H), 7.62˜7.65 (m, 2H), 7.42 (d, J=8 Hz, 2H), 7.22 (d, J=9 Hz, 2H), 6.89 (br t, J=6 Hz, 1H), 4.50 (s, 2H), 4.18 (q, J=7.5 Hz, 2H), 3.73 (dt, J=6 & 6 Hz, 2H), 3.64 (s, 2H), 2.65 (t, J=6 Hz, 2H), 1.80 (br d, J=13 Hz, 2H), 1.57˜1.65 (m, 4H), 1.29 (t, J=7.5 Hz, 3H), 1.07˜1.15 (m, 2H), 0.93˜1.00 (m, 1H), 0.88 (s, 9H). LC-MS: 2.55 min. (M+H=604.3).

### EXAMPLE 4

### N-[4-({TRANS-8-TERT-BUTYL-2-OXO-3-[4-(TRIFLUOROMETHOXY)PHENYL]-1,3-DIAZASPIRO[4.5]DEC-1-YL}METHYL)BENZOYL]-β-ALANINE

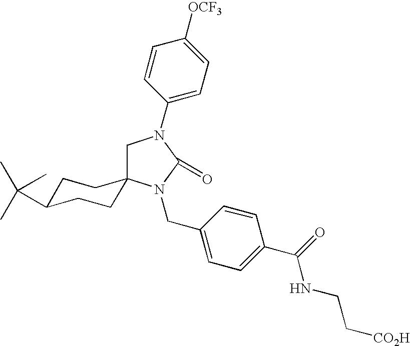

### Step A. Trans-tert-butyl N-[4-({8-tert-butyl-2-oxo-3-[4-(trifluoromethoxy)phenyl]-1,3-diazaspiro[4.5]dec-1-yl}methyl)benzoyl]-β-alaninate

The title compound was prepared from the product of Step C Example 2 and β-alanine tert-butyl ester hydrochloride using the procedure in Example 3. ¹H NMR (CDCl₃, 500 MHz) δ 7.71 (d, J=8.5 Hz, 2H), 7.62˜7.65 (m, 2H), 7.42 (d, J=8.5 Hz, 2H), 7.23 (d, J=9 Hz, 2H), 6.95 (br t, 1H), 4.50 (s, 2H), 3.69 (dt, J=6 & 6 Hz, 2H), 3.64 (s, 2H), 2.57 (t, J=6 Hz, 2H), 1.80 (br d, I=13.5 Hz, 2H), 1.57˜1.66 (m, 4H), 1.48 (s, 9H), 1.07˜1.15 (m, 2H), 0.93˜1.00 (m, 1H), 0.88 (s, 9H). LC-MS: 2.67 min. (M+H=632.3, base peak 576.3).

### Step B. N-[4-({trans-8-tert-butyl-2-oxo-3-[4-(trifluoromethoxy)phenyl]-1,3-diazaspiro[4.5]dec-1-yl}methyl)benzoyl]-β-alanine

The title compound was prepared from the product of Step A above using the same procedure from Step F Example 1. ¹H NMR (CD₃OD, 500 MHz) δ 7.77 (d, J=8 Hz, 2H), 7.68˜7.72 (m, 2H), 7.45 (d, J=8 Hz, 2H), 7.26 (d, J=8.5 Hz, 2H), 4.53 (s, 2H), 3.78 (s, 2H), 3.62 (t, J=7 Hz, 2H), 2.63 (t, J=7 Hz, 2H), 1.79 (br d, J=13 Hz, 2H), 1.64˜1.72 (m, 4H), 1.18˜1.27 (m, 2H), 1.00˜1.05 (m, 1H), 0.88 (s, 9H). LC-MS: 2.38 min. (M+H=576.3).

### EXAMPLE 5

### N-[4-({TRANS-8-TERT-BUTYL-2,4-DIOXO-3-[4-(TRIFLUOROMETHOXY)PHENYL]-1,3-DIAZASPIRO[4.5]DEC-1-YL}METHYL)BENZOYL]-β-ALANINE

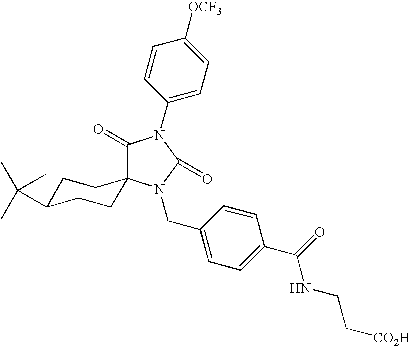

### Step A. Tert-butyl N-[4-({trans-8-tert-butyl-2,4-dioxo-3-[4-(trifluoromethoxy)phenyl]-1,3-diazaspiro[4.5]dec-1-yl}methyl)benzoyl]-β-alaninate

The title compound was prepared from the product from Step F Example 1 using the procedure in Step A Example 4. ¹H NMR (CDCl₃, 500 MHz) δ 7.76 (d, J=8.5 Hz, 2H), 7.54˜7.57 (m, 2H), 7.44 (d, J=8.5 Hz, 2H), 7.33 (d, J=9 Hz, 2H), 6.97 (br s, 1H), 4.64 (s, 2H), 3.71 (dt, J=6 & 6 Hz, 2H), 2.58 (t, J=6 Hz, 2H), 1.81˜1.95 (m, 6H), 1.67˜1.75 (m, 2H), 1.48 (s, 9H), 0.95˜1.02 (m, 1H), 0.89 (s, 9H). LC-MS: 2.57 min. (M+H=646.3, base peak 590.2).

### Step B. N-[4-({trans-8-tert-butyl-2,4-dioxo-3-[4-(trifluoromethoxy)phenyl]-1,3-diazaspiro[4,5]dec-1-yl}methyl)benzoyl]-β-alanine

The title compound was prepared from the product from A above using the procedure in Step B Example 4. ¹H NMR (CD₃OD, 500 MHz) δ 7.80 (d, J=8.5 Hz, 2H), 7.57˜7.60 (m, 2H), 7.49 (d, J=8 Hz, 2H), 7.41 (d, J=9 Hz, 2H), 3.63 (t, J=7 Hz, 2H), 2.64 (t, J=7 Hz, 2H), 1.77˜1.92 (m, 6H), 1.68˜1.73 (m, 2H), 1.04˜1.09 (m, 1H), 0.89 (s, 9H). LC-MS: 2.28 min. (M+H=590.2).

### EXAMPLE 6

### 4-({TRANS-9-TERT-BUTYL-2,4-DIOXO-3-[4-(TRIFLUOROMETHOXY)PHENYL]-1,3-DIAZASPIRO[5.5]UNDEC-1-YL}METHYL)-N-(1H-TETRAZOL-5-YL)BENZAMIDE

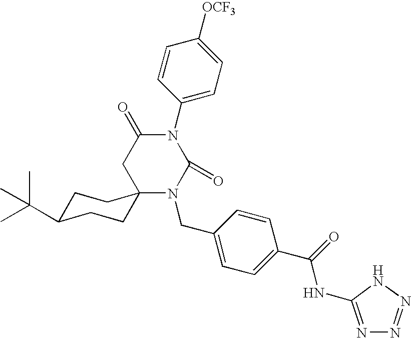

### Step A. (4-Tert-butylcyclohexylidene)acetonitrile

To a solution of 25.1 g diethyl (cyanomethyl)phosphonate in 300 mL anhydrous THF at −78° C. was added 22 mL 10 M n-butyl lithium in hexanes. After stirring for 30 minutes, a solution of 4-tert-butylcyclohexanone in 300 mL anhydrous THF was added. The reaction mixture was kept at −78° C. for 3 hours and then allowed to warm to room temperature slowly. It was poured into a mixture of ethyl acetate and 10% aqueous citric acid solution. The organic layer was separated and washed with saturated aq. NaHCO₃ and brine, concentrated under vacuum, and purified on silica gel with 1% ethyl acetate in hexanes to give the title compound as a white crystalline solid. ¹H NMR (CDCl₃, 500 MHz) δ 5.05 (s, 1H), 2.97˜3.02 (m, 1H), 2.41˜2.45 (m, 1H), 1.95˜2.21 (m, 4H), 1.11˜1.30 (m, 3H), 0.89 (s, 9H).

### Step B. (trans-1-amino-4-tert-butylcyclohexyl)acetonitrile

A mixture of 18 g product from Step A above, 140 mL 29% ammonia, and 100 mL methanol was heated at 100° C. for 25 hours in a pressure reactor. After cooling and venting, the reaction mixture was concentrated under vacuum. The resulting residue was dissolved in ethyl acetate and extracted with 6 N HCl. The aqueous layer was neutralized with KOH and extracted with ethyl acetate. The crude product from ethyl acetate was purified on silica gel using 50 to 100% ethyl acetate in hexanes to give the title compound and the less polar cis isomer, both as white crystalline solids. The isomers were assigned based on homonuclear decoupling and NOE experiments. ¹H NMR of the title compound (CDCl₃, 500 MHz) δ 2.50 (s, 2H), 1.83˜1.88 (m, 2H), 1.73˜1.78 (m, 2H), 1.37˜1.45 (m, 2H), 0.95˜1.05 (m, 3H), 0.88 (s, 9H). LC-MS: 1.22 min. (M+H=195.2).

### Step C. Trans-9-tert-butyl-4-imino-3-[4-(trifluoromethoxy)phenyl]-1,3-diazaspiro[5.5]-undecan-2-one

A mixture of 4.5 g of the product from Step B above and 4.7 g 4-(trifluoromethoxy)-phenyl isocyanate in 200 mL benzene was stirred at room temperature for 1 day. To this mixture was added 926 mg 60% sodium hydride oil dispersion. After stirring 8 hours at room temperature, the reaction mixture was poured into saturated ammonium chloride and extracted with ethyl acetate. The crude product from the organic phase was purified on silica gel with 2:1 hexanes and ethyl acetate to give the title compound as a white solid. ¹H NMR (CDCl₃, 500 MHz) δ 7.31˜7.34 (m, 2H), 7.12 (d, J=8.5 Hz, 2H), 6.79 (br s, 1H), 5.08 (br s, 1H), 3.22 (s, 2H), 2.41 (br d, J=13 Hz, 2H), 1.71˜1.75 (m, 2H), 1.40˜1.47 (m, 2H), 0.99˜1.07 (m, 3H), 0.86 (s, 9H).

### Step D. Trans-9-tert-butyl-3-[4-(trifluoromethoxy)phenyl]-1,3-diazaspiro[5.5]undecane-2,4-dione

A mixture of 4.0 g of the product from Step C above and 300 mL 1.5 M hydrochloric acid in 200 mL ethanol was refluxed for 10 hours. The organic solvent was removed under reduced pressure. The solid from the residue was filtered and washed with water, 1 N aq. NaOH, dried, and purified on silica gel using 2:1 hexanes and ethyl acetate to give the title compound as white solid.

### Step E. Tert-butyl 4-({trans-9-tert-butyl-2,4-dioxo-3-[4-(trifluoromethoxy)phenyl]-1,3-diazaspiro[5.5]undec-1-yl}methyl)benzoate

The title compound was prepared from the product of Step D above using the same procedure described in Step E Example 1 except 2:1 hexanes and ethyl acetate was used for column purification. ¹H NMR (CDCl₃, 500 MHz) δ 7.98 (d, J=8.5 Hz, 2H), 7.38 (d, J=8 Hz, 2H), 7.32 (d, J=8.5 Hz, 2H), 7.25˜7.28 (m, 2H), 4.83 (s, 2H), 2.97 (s, 2H), 1.73˜1.84 (m, 6H), 1.61 (s, 9H), 1.16˜1.25 (m 2H), 0.96˜1.02 (m, 1H), 0.87 (s, 9H). LC-MS: 2.68 min. (M+H=589.2).

### Step F. 4-({trans-9-tert-butyl-2,4-dioxo-3-[4-(trifluoromethoxy)phenyl]-1,3-diazaspiro-[5.5]undec-1-yl}methyl)benzoic acid

To a solution of 1.02 g product from Step E above in 16 mL dichloromethane was added 4 mL trifluoroacetic acid. The reaction mixture was concentrated under vacuum after one hour to give the title compound as a white solid. LC-MS: 2.28 min. (M+H=533.2).

### Step G. 4-({Trans-9-tert-butyl-2,4-dioxo-3-[4-(trifluoromethoxy)phenyl]-1,3-diazaspiro-[5.5]undec-1-yl}methyl)-N-(1H-tetrazol-5-yl)benzamide

A solution of 100 mg product from Step F above, 54 mg EDC, 38 mg HOBt, 65.5 μL DIEA in 5 mL DMF was stirred at room temperature for 30 minutes. 5-Aminotetrazole monohydrate (25 mg) was added and the mixture stirred for additional 12 hours. It was purified on RP-HPLC using acetonitrile and water mixture with 0.1% (v/v) TFA. The pure product fractions were pooled, neutralized with ammonia in methanol, purified on silica gel, concentrated under vacuum, and lyophilized to give the pure title compound. ¹H NMR (CD₃OD, 500 MHz) δ 8.03 (d, J=8.5 Hz, 2H), 7.57 (d, J=8 Hz, 2H), 7.35˜7.39 (m, 4H), 4.92 (s, 2H), 3.08 (s, 2H), 1.83˜1.93 (m, 4H), 1.77 (br d, J=12.5 Hz, 2H), 1.21˜1.30 (m, 2H), 1.06˜1.13 (m, 1H), 0.88 (s, 9H). LC-MS: 2.18 min. (M+H=600.2).

### EXAMPLE 7

### 4-({TRANS-9-TERT-BUTYL-2-OXO-3-[4-(TRIFLUOROMETHOXY)PHENYL]-1,3-DIAZASPIRO[5.5]UNDEC-1-YL}METHYL)-N-(1H-TETRAZOL-5-YL)BENZAMIDE

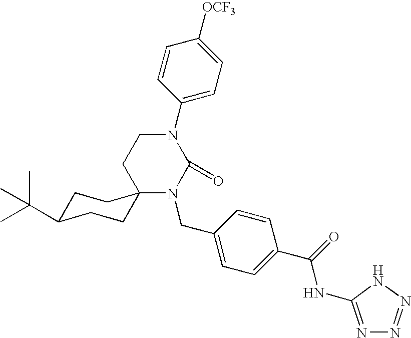

### Step A. Trans-9-tert-butyl-3-[4-(trifluoromethoxy)phenyl]-1,3-diazaspiro[5.5]undecan-2-one

To a suspension of 2.0 g product from Step D Example 6 and 1.0 g anhydrous aluminum chloride in 20 mL ether was added 5.02 mL 1 M LAH in ether. After 4 hours, it was poured into saturated ammonium chloride and extracted with ethyl acetate. The organic layer was washed with water and saturated brine, evaporated under vacuum, and purified on silica gel using 2:1 hexanes and ethyl acetate to give the title compound as a white solid. ¹H NMR (CDCl₃, 500 MHz) δ 7.34˜7.37 (m, 2H), 7.20 (d, J=8.5 Hz, 2H), 4.57 (br s, 1H), 3.64˜3.67 (m, 2H), 1.99˜2.01 (m, 2H), 1.94˜1.98 (m, 2H), 1.72˜1.77 (m, 2H), 1.39˜1.45 (m, 2H), 1.13˜1.22 (m, 2H), 1.06 (tt, J=3 & 12 Hz, 1H), 0.89 (s, 9H). LC-MS: 2.35 min. (M+H=385.2).

### Step B. Tert-butyl 4-({trans-9-tert-butyl-2-oxo-3-[4-(trifluoromethoxy)phenyl]-1,3-diazaspiro[5.5]undec-1-yl}methyl)benzoate

The title compound was prepared from the product of the Step A above using the same procedure from Step E Example 1. ¹H NMR (CDCl₃, 500 MHz) δ 7.94 (d, 8 Hz, 2H), 7.34˜7.40 (m, 4H), 7.20 (d, 8.5 Hz, 2H), 4.73 (br s, 2H), 3.70 (t, 6 Hz, 2H), 2.17 (t, J=7 Hz, 2H), 1.87˜1.98 (m, 6H), 1.76 (s, 9H), 1.13˜1.21 (m, 2H), 0.97˜1.03 (m, 1H), 0.86 (s, 9H).

### Step C. 4-({Trans-9-tert-butyl-2-oxo-3-[4-(trifluoromethoxy)phenyl]-1,3-diazaspiro[5.5]undec-1-yl}methyl)benzoic acid

The title compound was prepared from the product of the Step B above using the same procedure from Step F Example 1. LC-MS: 2.47 min. (M+H=519.2).

### Step D. 4-({Trans-9-tert-butyl-2-oxo-3-[4-(trifluoromethoxy)phenyl]-1,3-diazaspiro[5.5]undec-1-yl}methyl)-N-(1H-tetrazol-5-yl)benzamide

The title compound was prepared from the product of the Step C above using the same procedure from Step G Example 6. ¹H NMR (CD₃OD, 500 MHz), 7.99 (d, J=8 Hz, 2H), 7.475 (d, J=8 Hz, 2H), 7.42˜7.45 (m, 2H), 7.28 (d, J=8.5 Hz, 2H), 4.79 (s, 2H), 3.76 (t, J=6 Hz, 2H), 2.25 (t, J=6 Hz, 2H), 1.69˜1.87 (m, 6H), 1.24˜1.33 (m, 2H), 1.05˜1.11 (m, 1H), 0.88 (s, 9H). LC-MS: 2.36 min. (M+H=586.2).

### EXAMPLE 8

### N-[4-({TRANS-9-TERT-BUTYL-2,4-DIOXO-3-[4-(TRIFLUOROMETHOXY)-PHENYL]-1,3-DIAZASPIRO[5.5]UNDEC-1-YL}METHYL)BENZOYL]-β-ALANINE

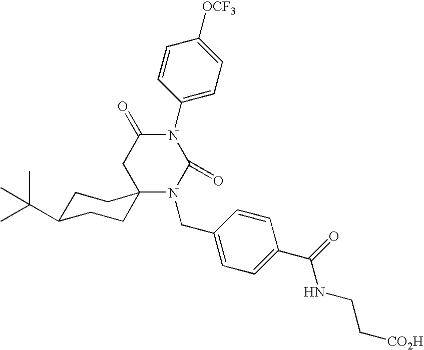

### Step A. Tert-butyl N-[4-({trans-9-tert-butyl-2,4-dioxo-3-[4-(trifluoromethoxy)phenyl]-1,3-diazaspiro[5.5]undec-1-yl}methyl)benzoyl]-β-alaninate

The title compound was prepared from the intermediate obtained from Step F Example 6 using procedure in Step A Example 4. LC-MS: 2.43 min. (M+H=660.2, base peak 604.1).

### Step B. N-[4-({trans-9-tert-butyl-2,4-dioxo-3-[4-(trifluoromethoxy)phenyl]-1,3-diazaspiro[5.5]undec-1-yl}methyl)benzoyl]-β-alanine

The title compound was prepared from the intermediate from Step A above using procedure in Step B Example 4. ¹H NMR (CD₃OD, 500 MHz) δ 7.79 (d, J=8.5 Hz, 2H), 7.47 (d, J=8.5 Hz, 2H), 7.34˜7.39 (m, 4H), 4.88 (s, 2H), 3.63 (t, J=7 Hz, 2H), 3.06 (s, 2H), 2.64 (t, J=7 Hz, 2H), 1.81˜1.91 (m, 4H), 1.76 (d, J=13 Hz, 2H), 1.19˜1.28 (m, 2H), 1.03˜1.10 (m, 1H), 0.99 (s, 9H). LC-MS: 2.13 min. (M+H=604.2).

### EXAMPLE 9

### N-[4-({TRANS-9-TERT-BUTYL-2-OXO-3-[4-(TRIFLUOROMETHOXY)PHENYL]-1,3-DIAZASPIRO[5.5]UNDEC-1-YL}METHYL)BENZOYL]-β-ALANINE

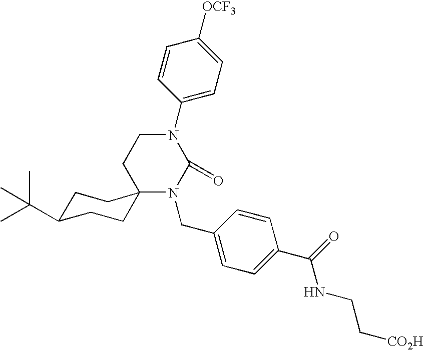

### Step A. Tert-butyl N-[4-({trans-9-tert-butyl-2-oxo-3-[4-(trifluoromethoxy)phenyl]-1,3-diazaspiro[5.5]undec-1-yl}methyl)benzoyl]-(3-alaninate

The title compound was prepared from the intermediate obtained from Step C Example 7 using procedure in Step A, Example 4. LC-MS: 2.59 min. (M+H=646.3, base peak 590.3).

### Step B. N-[4-({trans-9-tert-butyl-2-oxo-3-[4-(trifluoromethoxy)phenyl]-1,3-diazaspiro[5.5]undec-1-yl}methyl)benzoyl]-β-alanine

The title compound was prepared from the intermediate from Step A above using procedure in Step B Example 4. ¹H NMR (CD₃OD, 500 MHz) & 7.76 (d, J=8.5 Hz, 2H), 7.41˜7.44 (m, 2H), 7.38 (d, J=8.5 Hz, 2H), 7.28 (d, J=8.5 Hz, 2H), 4.75 (s, 2H), 3.74 (t, J=6 Hz, 2H), 3.62 (t, J=7 Hz, 2H), 2.63 (t, J=7 Hz, 2H), 2.23 (t, J=6 Hz, 2H), 1.67˜1.85 (m, 6H), 1.22˜1.31 (m, 2H), 1.03˜1.10 (m, 1H), 0.87 (s, 9H). LC-MS: 2.31 min. (M+H=590.3).

### EXAMPLE 10

### METHYL (2R)-3-{[4-({TRANS-9-TERT-BUTYL-2-OXO-3-[4-(TRIFLUOROMETHOXY)PHENYL]-1,3-DIAZASPIRO[5.5]UNDEC-1-YL)METHYL)BENZOYL]AMINO}-2-HYDROXYPROPANOATE

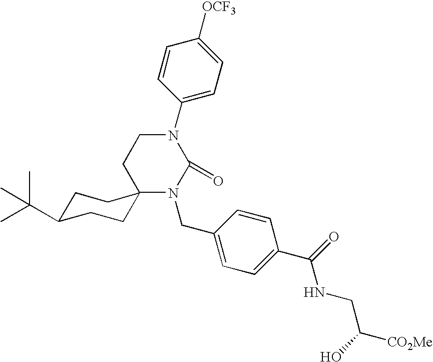

### Step A. [(4R)-2,2-Dimethyl-5-oxo-1,3-dioxolan-4-yl]acetic acid

A solution of 25.05 g D-malic acid and 68.1 g 2,2-dimethoxypropane in 200 mL toluene was refluxed for 2 hours under nitrogen. The solvent was removed under reduced pressure to give the title compound as a white solid. ¹H NMR (CDCl₃, 500 MHz) δ 4.76 (dd, 3.9 & 6.6 Hz, 1H), 3.02 (dd, 3.9 & 17.2 Hz, 1H), 2.88 (dd, 6.6 & 17.2 Hz, 1H), 1.64 (s, 3H), 1.59 (s, 3H).

### Step B. Benzyl [(4R)-2,2-dimethyl-5-oxo-1,3-dioxolan-4-yl]methylcarbamate

A solution of 5.25 g intermediate from Step A above, 8.88 g diphenylphosphoryl azide, and 3.34 g triethyl amine in 100 mL toluene was refluxed under nitrogen for 75 minutes. Benzyl alcohol (2.92 g) was added and reflux continued for additional 15 hours. The reaction mixture was cooled, diluted with ethyl acetate, washed with 5% aq. NaHCO₃ and saturated brine, dried over anhydrous Na₂SO₄, and evaporated under vacuum to give a crude product. It was purified on silica gel with 20˜45% ethyl acetate in hexanes to give the title compound as a yellow oil. ¹H NMR (CDCl₃, 500 MHz) δ 7.33˜7.40 (m, 5H), 5.175 (d, 12.1 Hz, 1H), 5.11 (d, 11.9 Hz, 1H), 4.50˜4.52 (m, 1H), 3.69˜3.75 (m, 1H), 3.60˜3.66 (m, 1H), 1.59 (s, 3H), 1.57 (s, 3H).

### Step C. Methyl (2R)-3-amino-2-hydroxypropanoate hydrochloride

Benzyl [(4R)-2,2-dimethyl-5-oxo-1,3-dioxolan-4-yl]methylcarbamate (7.76 g) prepared by the method described in Step B above was dissolve in methanol (70 mL) with 0.62 g 10% Pd/C. A 1 M HCl in ether solution was added (25 mL). This mixture was hydrogenated using a hydrogen balloon for 22 hours. The reaction mixture was purged with nitrogen, filtered though a pad of Celite, and evaporated under vacuum to give the title compound as a yellowish solid. ¹H NMR (CD₃OD, 500 MHz) δ 4.45 (dd, J=4 and 8 Hz, 1H), 3.82 (s, 3H), 3.31 (dd, 1H), 3.15 (dd, J=8 and 13 Hz, 1H).

### Step D. Methyl (2R)-3-[4-({trans-9-tert-butyl-2-oxo-3-[4-(trifluoromethoxy)phenyl]-1,3-diazaspiro[5.5]undec-1-yl}methyl)benzoyl]amino}-2-hydroxypropanoate

The title compound was prepared from the intermediates from Step C above and Step C Example 7 using procedure described in Step A Example 4. ¹H NMR (CDCl₃, 500 MHz) δ 7.70 (d, J=8 Hz, 2H), 7.36˜7.39 (m, 4H), 7.20 (d, J=8.5 Hz, 2H), 6.51 (t, J=6 Hz, 1H), 4.72 (s, 2H), 4.40˜4.42 (m, 1H), 3.80˜3.89 (m, 2H), 3.70 (t, J=6 Hz, 2H), 2.18 (t, J=6 Hz, 2H), 1.70˜1.78 (m, 6H), 1.15˜1.24 (m, 2H), 0.98˜1.04 (m, 1H), 0.87 (s, 9H). LC-MS: 2.63 min. (M+H=620.3).

### EXAMPLE 11

### (2R)-3-{[4-({TRANS-9-TERT-BUTYL-2-OXO-3-[4-(TRIFLUOROMETHOXY)-PHENYL]-1,3-DIAZASPIRO[5.5]UNDEC-1-YL}METHYL)BENZOYL]AMINO}-2-HYDROXYPROPANOIC ACID

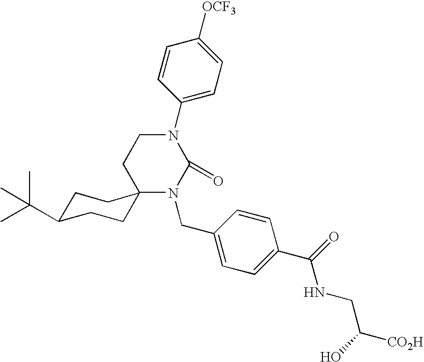

The intermediate from Step D Example 10 (19 mg) was dissolved in 3 mL methanol and treated with 65 μL 5 N aqueous sodium hydroxide at room temperature for 4 hours. Solvents were removed under reduced pressure and the residue was purified on reverse-phase HPLC to give the title compound as a white solid after lyophilization. LC-MS: 2.54 min. (M+H=606.4).

### EXAMPLE 12

### 4-({TRANS-9-TERT-BUTYL-2-OXO-3-[4-(TRIFLUOROMETHOXY)PHENYL]-1,3-DIAZASPIRO[5.5]UNDEC-1-YL}METHYL)-N-(1H-TETRAZOL-5-YLMETHYL)BENZAMIDE

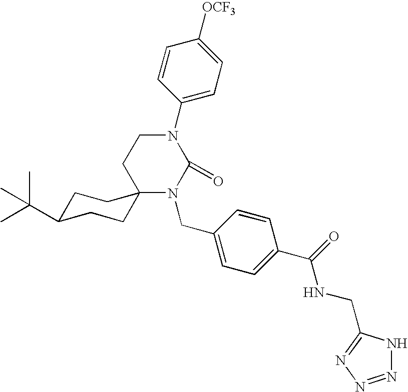

The title compound was prepared from the intermediate from Step C Example 7 and 5-aminomethyltetrazole using procedure in Step D Example 7. ¹H NMR (DMSO-d₆, 600 MHz) δ 9.12 (t, J=5.7 Hz, 1H), 7.79 (d, J=8.4 Hz, 2H), 7.41˜7.43 (m, 2H), 7.33 (d, J=8.4 Hz, 2H), 7.28 (d, J=8.4 Hz, 2H), 4.71 (d, J=5.4 Hz, 2H), 4.62 (br s, 2H), 3.65 (t, J=6 Hz, 2H), 2.09 (t, J=5.4 Hz, 2H), 1.66˜1.71 (m, 2H), 1.53˜1.61 (m, 4H), 1.09˜1.16 (m, 2H), 0.97˜1.02 (m, 1H), 0.79 (s, 9H). LC-MS: 2.53 min. (M+H=600.4).

Following the procedures outlined for Examples 1-12 the compounds listed in Tables 2-4 were prepared.

TABLE 2

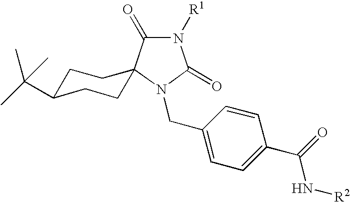

| Example | R¹ | R² | LC-MS, min. (M + H) |
|---|---|---|---|
| 13 | Ph | 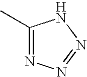 | 1.92 (502.3) |
| 14 | Ph | 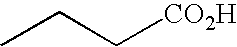 | 1.84 (506.3) |
| 15 | C₆H₁₁ | 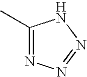 | 2.30 (508.3) |
| 16 | C₆H₁₁ | 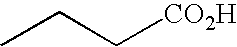 | 2.25 (512.2) 31758-245 |
| 17 | t-Bu | 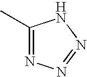 | 2.22 (482.2) 31758-246 |
| 18 | t-Bu | 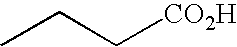 | 2.15 (486.2) 31758-248 |
| 19 | i-Pr | 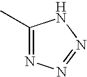 | 2.07 (468.2) 31758-249 |
| 20 | i-Pr | 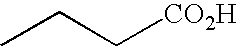 | 2.02 (472.2) 31758-251 |

TABLE 3

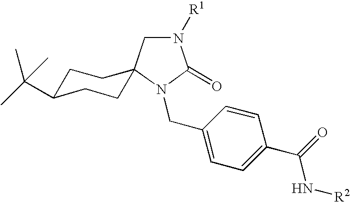

| Example | R¹ | R² | LC-MS, min. (M + H) |
|---|---|---|---|
| 21 | Ph | 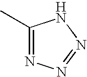 | 2.00 (488.3) |
| 22 | Ph | 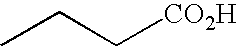 | 1.93 (492.3) |
| 23 | C₆H₁₁ | 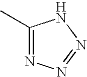 | 3.96 (494.4) |
| 24 | C₆H₁₁ | 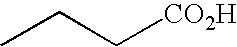 | 3.84 (498.4) |
| 25 | t-Bu | 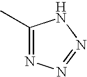 | 2.03 (468.3) 31758-234 |
| 26 | t-Bu | 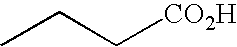 | 3.67 (472.4) |
| 27 | i-Pr | 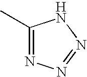 | 2.06 (454.2) |
| 28 | i-Pr | 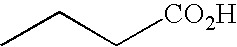 | 2.01 (458.3) |
| 29 | Ph | 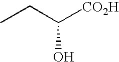 | 2.36 (508.2) |
| 30 | C₆H₁₁ | 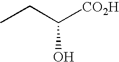 | 2.47 (514.3) |
| 31 | t-Bu | 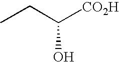 | 2.35 (488.2) |
| 32 | i-Pr | 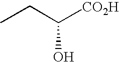 | 2.20 (474.2) |
| 33 | 2-ClPh | 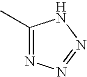 | 3.89 (522.1) |
| 34 | 3-ClPh | 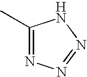 | 4.16 (522.1) |
| 35 | 4-ClPh | 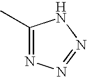 | 2.61 (522.3) |
| 36 | 2,4-diClPh | 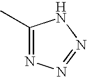 | 2.67 (556.3/558.1) |
| 37 | 3,5-diClPh | 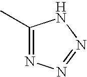 | 4.51 (556.1/558.1) |
| 38 | 2-ClPh | 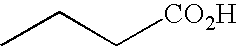 | 2.46 (526.3) |
| 39 | 3-ClPh | 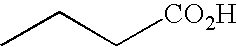 | 2.59 (526.3) |
| 40 | 4-ClPh | 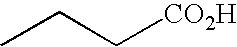 | 2.59 (526.3) |
| 41 | 2,4-diClPh | 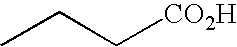 | 2.62 (560.3/562.3) |
| 42 | 3,5-diClPh | 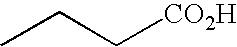 | 2.77 (560.3/562.3) |
| 43 | 2-ClPh | 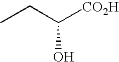 | 2.38 (542.4) |
| 44 | 3-ClPh | 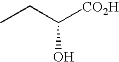 | 2.38 (542.3) |
| 45 | 4-ClPh | 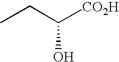 | 2.51 (542.4) |
| 46 | 2,4-diClPh | 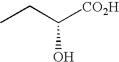 | 2.55 (576.3/578.3) |
| 47 | 3,5-diClPh | 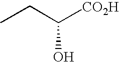 | 2.69 (576.3/578.3) |
| 48 | 2-ClPh | 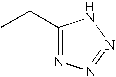 | 2.40 (536.3) |
| 49 | 3-ClPh | 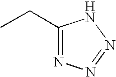 | 2.52 (536.4) |
| 50 | 4-ClPh | 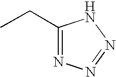 | 2.63 (536.4) |
| 51 | 2,4-diClPh | 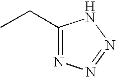 | 2.40 (570.3/572.3) |
| 52 | 3,5-diClPh | 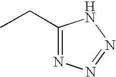 | 2.70 (570.3/572.3) |
| 53 | 4-FPh | 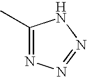 | 2.52 (506.3) |
| 54 | 4-CF₃Ph | 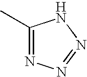 | 2.65 (556.3) |
| 55 | 4-MePh | 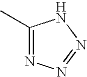 | 2.60 (502.4) |
| 56 | 4-BrPh | 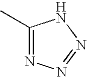 | 2.64 (566/568.2) |
| 57 | 4-FPh | 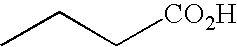 | 2.46 (510.4) |
| 58 | 4-CF₃Ph | 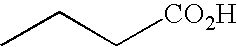 | 2.60 (560.3) |
| 59 | 4-MePh | 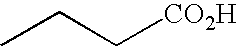 | 2.55 (506.4) |
| 60 | 4-BrPh | 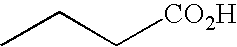 | 2.61 (570/572.3) |
| 61 | 4-FPh | 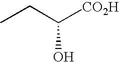 | 3.65 (526.3) |
| 62 | 4-CF₃Ph | 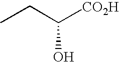 | 4.00 (576.3) |
| 63 | 4-MePh | 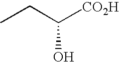 | 2.42 (522.4) |
| 64 | 4-BrPh | 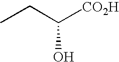 | 3.97 (586.3/588) |
| 65 | 4-FPh | 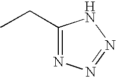 | 2.38 (520.4) |
| 66 | 4-CF₃Ph | 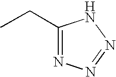 | 2.55 (570.4) |
| 67 | 4-MePh | 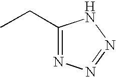 | 2.47 (516.4) |
| 68 | 4-BrPh | 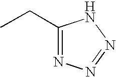 | 2.56 (580/582.3) |
| 69 | 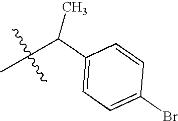 | 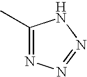 | 4.01 (594/596.3) |
| 70 | 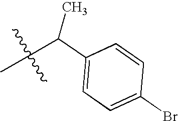 | 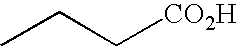 | 3.97 (598/600.3) |
| 71 | 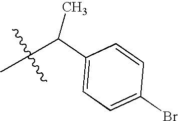 | 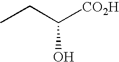 | 3.84 (614/616.3) |
| 72 | 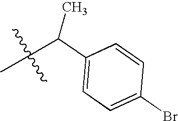 | 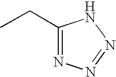 | 3.94 (608/610.3) |
| 73 | 4-CH₃SO₂Ph | 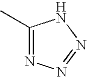 | 3.49 (566.5, base peak 365.3) |
| 74 | 3-CF₃OPh | 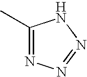 | 4.14 (572.5) |
| 75 | 3-CF₃SPh | 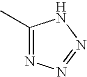 | 4.16 (588.3) |
| 76 | 3,4-F₂Ph | 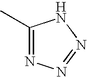 | 4.00 (524.4) |
| 77 | 2,5-Cl₂Ph | 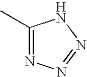 | 4.05 (556.4/558.3) |
| 78 | 2,4-Cl₂PhCH₂ | 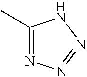 | 4.18 (570.4/572.4) |
| 79 | 3,4-Cl₂PhCH₂ | 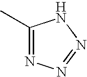 | 4.10 (570.3/572.3) |
| 80 | 3-FPhCH₂ | 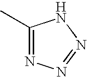 | 3.79 (520.4) |
| 81 | 2,4-F₂Ph | 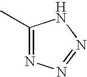 | 4.18 (524.4) |
| 82 | 4-CH₃SO₂Ph | 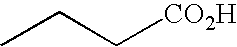 | 3.92 (570.3) |
| 83 | 3-CF₃OPh | 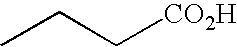 | 4.52 (576.4) |
| 84 | 3-CF₃SPh | 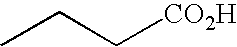 | 4.16 (592.3) |
| 85 | 3,4-F₂Ph | 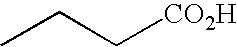 | 4.35 (528.3, base peak 569.4) |
| 86 | 2,5-Cl₂Ph | 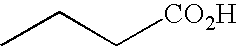 | 4.48 (560.3/562, base peak 601.3/603) |
| 87 | 2,4-Cl₂PhCH₂ | 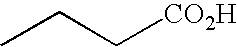 | 4.52 (574.2/576.3) |
| 88 | 3,4-Cl₂PhCH₂ | 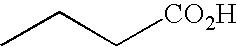 | 4.59 (574.3/576.3) |
| 89 | 3-FPhCH₂ | 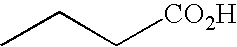 | 4.25 (524.3) |
| 90 | 2,4-F₂Ph | 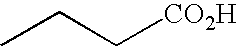 | 4.26 (528.3) |

TABLE 4

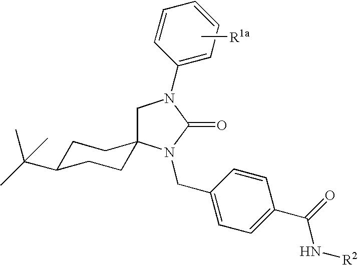

| Example | R^{1a} | R² | LC-MS, min. (M + H) |
|---|---|---|---|
| 91 | 4-NMe₂ | 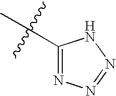 | 2.93(531.2) |
| 92 | 4-NMe₂ | 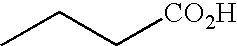 | 3.29(535.2) |
| 93 | 3,5-bisCF₃ | 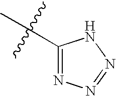 | 4.28(624.2) |
| 94 | 3,5-bisCF₃ | 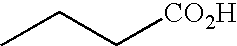 | 4.61(628.2) |
| 95 | 3-F,5-CF₃ | 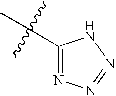 | 4.15(574.2) |
| 96 | 3-F,5-CF₃ | 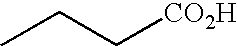 | 4.52(578.2) |
| 97 | 4-Py | 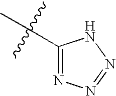 | 1.97(565.2) |
| 98 | 4-Py | 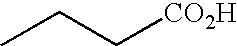 | 1.93(569.2) |

### EXAMPLE 99

### 4-({8-BENZYL-2,4-DIOXO-3-[4-(TRIFLUOROMETHOXY)PHENYL]-1,3,8-TRIAZASPIRO[4.5]DEC-1-YL}METHYL)-N-(1H-TETRAZOL-5-YL)BENZAMIDE

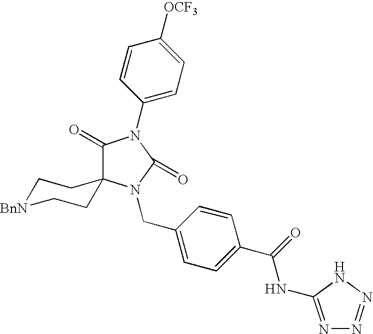

### Step A. 4-Amino-1-benzylpiperidine-4-carbonitrile

A mixture of 100 g 1-benzyl-4-piperidone, 36.2 g potassium cyanide, 29.7 g ammonium chloride and 500 mL each of methanol and water was stirred for two days at room temperature. The reaction mixture was concentrated under vacuum. This crude product from ethyl acetate workup was purified on silica gel column to give the title compounds as a yellow solid. ¹H NMR (CDCl₃, 500 MHz) δ 7.31˜7.36 (m, 4H), 7.26˜7.31 (m, 1H), 3.56 (s, 2H), 2.82˜2.86 (m, 2H), 2.34˜2.39 (m, 2H), 1.98˜2.03 (m, 2H), 1.76˜1.82 (m, 2H). LC-MS: 0.50 min. (M+H=216.3).

### Step B. N-(1-benzyl-4-cyanopiperidin-4-yl)-N′-[4-(trifluoromethoxy)phenyl]urea

A solution of 9.6 g 4-amino-1-benzylpiperidine-4-carbonitrile and 10 g 4-(trifluoromethoxy)phenyl isocyanate in 400 mL benzene was stirred at room temperature for 10 hours. The resulting white precipitate was filtered, washed with hexanes, and dried to give the title compound as a white solid. ¹H NMR (CDCl₃, 500 MHz) δ 8.25 (br s, 1H), 7.27˜7.35 (m, 7H), 7.04 (d, J=8.5 Hz, 2H), 6.10 (br s, 1H), 3.55 (s, 2H), 2.75 (br s, 2H), 2.43˜2.48 (m, 2H), 2.34 (br d, J=13 Hz, 2H), 1.90˜1.95 (m, 2H). LC-MS: 1.52 min. (M+H=419.1).

### Step C. 8-Benzyl-4-imino-3-[4-(trifluoromethoxy)phenyl]-1,3,8-triazaspiro[4.5]decan-2-one

To a suspension of 16.5 g N-(1-benzyl-4-cyanopiperidin-4-yl)-N′-[4-(trifluoromethoxy)phenyl]urea in 300 mL toluene was added 1.60 g 60% sodium hydride oil dispersion. The reaction mixture was stirred at room temperature for 10 hours and worked up with saturated ammonium chloride and ethyl acetate. The organic layer was concentrated under vacuum to give the title compound as a white solid. ¹H NMR (CDCl₃, 500 MHz) δ 7.56 (d, J=8 Hz, 2H), 7.28˜7.40 (m, 7H), 6.58 (br s, 1H), 6.29 (br s, 1H), 3.57 (s, 2H), 3.00 (d, J=12 Hz, 2H), 2.29˜2.36 (m, 1H), 2.14˜2.20 (m, 2H), 2.04˜2.10 (m, 1H), 1.81 (d, J=12 Hz, 2H). LC-MS: 1.13 min. (M+H=419.2).

### Step D. 8-Benzyl-3-[4-(trifluoromethoxy)phenyl]-1,3,8-triazaspiro[4.5]decane-2,4-dione

A suspension of 14.5 g of the product from Step C above and 300 mL each of 6 M hydrochloric acid and ethanol was refluxed for 5 hours. The reaction mixture was cooled to room temperature and a fine needle collected by filtration. The solid was partitioned between methylene chloride and 1 N aq. potassium hydroxide. The organic layer was separated and concentrated under vacuum to give the title compound as white solid. ¹H NMR (CDCl₃, 500 MHz) δ 7.53˜7.56 (m, 2H), 7.33˜7.37 (m, 6H), 7.27˜7.31 (m, 1H), 7.05 (br s, 1H), 3.57 (s, 2H), 2.93˜3.00 (m, 2H), 2.24˜2.31 (m, 4H), 1.74˜1.81 (m, 21).

### Step E. Tert-butyl 4-({8-benzyl-2,4-dioxo-3-[4-(trifluoromethoxy)phenyl]-1,3,8-triazaspiro[4.5]dec-1-yl}methyl)benzoate

A solution of 0.7 g product from Step D above in 30 mL DMF was evacuated under high vacuum for 10 minutes. Sodium hydride (60% oil dispersion, 74 mg) was added and the mixture stirred for 20 minutes. Tert-butyl 4-(bromomethyl)benzoate (0.5 g) was then added and the resulting mixture stirred at room temperature for 8 hours. After working up with saturated aqueous ammonium chloride and ethyl acetate, the crude product was purified on silica gel with 10 to 50% ethyl acetate in hexanes to give the title compound as a white solid. ¹H NMR (CDCl₃, 500 MHz) δ 7.98 (d, J=8 Hz, 2H), 7.54˜7.57 (m, 2H), 7.41 (d, J=8 Hz, 2H), 7.30˜7.35 (m, 6H), 4.68 (s, 2H), 3.59 (s, 2H), 2.75˜2.84 (m, 4H), 2.01˜2.07 (m, 2H), 1.72 (d, J=13.5 Hz, 2H), 1.60 (s, 9H).

### Step F. 4-({8-Benzyl-2,4-dioxo-3-[4-(trifluoromethoxy)phenyl]-1,3,8-triazaspiro[4.5]dec-1-yl}methyl)benzoic acid

A solution of 0.5 g product from Step E above in 20 mL 30% (v/v) trifluoroacetic acid in dichloromethane was stirred at room temperature for 4 hours and concentrated under vacuum to give the title compound as a solid. ¹H NMR (CDCl₃, 500 MHz) δ 7.96 (d, J=8 Hz, 2H), 7.53˜7.56 (m, 2H), 7.37˜7.46 (m, 9H), 4.72 (s, 2H), 4.25 (s, 2H), 3.56˜3.69 (m, 4H), 2.68˜2.74 (m, 2H), 1.86 (d, J=14.5 Hz, 2H). LC-MS: 1.91 min. (M+H=554.1).

### Step G. 4-({8-Benzyl-2,4-dioxo-3-[4-(trifluoromethoxy)phenyl]-1,3,8-triazaspiro[4.5]-dec-1-yl}methyl)-N-(1H-tetrazol-5-yl)benzamide

The title compound (236 mg) was prepared from 200 mg 4-({8-benzyl-2,4-dioxo-3-[4-(trifluoromethoxy)phenyl]-1,3,8-triazaspiro[4.5]dec-1-yl}methyl)benzoic acid using procedure described in Step G Example 6. ¹H NMR (DMSO-d₆, 500 MHz) δ 8.07 (d, J=8 Hz, 2H), 7.62˜7.65 (m, 2H), 7.57 (d, J=8 Hz, 2H), 7.53 (d, J=8.5 Hz, 2H), 7.47˜7.49 (m, 2H), 7.41˜7.45 (m, 3H), 4.67 (s, 2H), 4.23 (br s, 2H), 3.31 (br s, 4H plus water peak), 2.15˜2.25 (m, 4H). LC-MS: 1.89 min. (M+H=621.2).

### EXAMPLE 100

### 4-({8-BENZYL-2-OXO-3-[4-(TRIFLUOROMETHOXY)PHENYL]-1,3,8-TRIAZASPIRO[4.5]DEC-1-YL}METHYL)-N-(1H-TETRAZOL-5-YL)BENZAMIDE

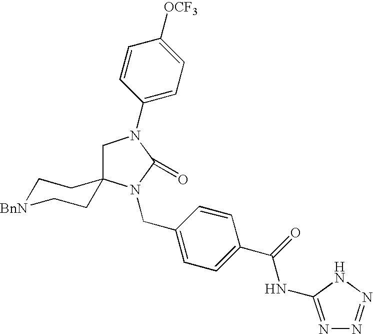

### Step A. 8-Benzyl-3-[4-(trifluoromethoxy)phenyl]-1,3,8-triazaspiro[4.5]decan-2-one

To a suspension of 1 g product from Step D of the previous Example and 480 mg anhydrous aluminum chloride in 100 mL ether was added slowly 2.4 mL 1 M LAH solution in ether. After stirring at room temperature for 10 hours, LC-MS indicated there was about 4:1 ratio of product and starting material. More AlCl₃ and LAH (1.5 equiv. each) were added and the mixture stirred at room temperature for additional 10 hours. The reaction mixture was worked up using 1 N aq. KOH and ethyl acetate. The crude product was combined with that from another run from 10 g of 8-benzyl-3-[4-(trifluoromethoxy)phenyl]-1,3,8-triazaspiro[4.5]decane-2,4-dione and purified on silica gel using 0˜2% methanol in ethyl acetate to give the title compound. ¹H NMR (CDCl₃, 500 MHz) δ 7.58˜7.61 (m, 2H), 7.32˜7.37 (m, 4H), 7.27˜7.31 (m, 1H), 7.21 (d, J=9 Hz, 2H), 5.6 (br s, 1H), 3.67 (s, 2H), 3.56 (s, 2H), 2.52 (br s, 4H), 1.85 (t, J=5.5 Hz, 4H).

Step B. Methyl 4-({8-benzyl-2-oxo-3-[4-(trifluoromethoxy)phenyl]-1,3,8-triazaspiro-[4.5]dec-1-yl}methyl)benzoate The title compound (2.65 g white solid) was prepared from 2 g 8-benzyl-3-[4-(trifluoromethoxy)phenyl]-1,3,8-triazaspiro[4.5]decan-2-one, 1.25 g methyl 4-bromomethylbenzoate, 217 mg 60% NaH, and 100 mL DMF using the procedure in Step E of the previous Example. ¹H NMR (CDCl₃, 500 MHz) δ 7.99 (d, J=8.0 Hz, 2H), 7.62˜7.66 (m, 2H), 7.43 (d, J=8.5 Hz, 2H), 7.26˜7.35 (m, 5H), 7.23 (d, J=9 Hz, 2H), 4.54 (s, 2H), 3.91 (s, 3H), 3.69 (s, 2H), 3.52 (s, 2H), 2.89 (br d, J=12.0 Hz, 2H), 2.04˜2.08 (m, 2H), 1.92˜1.98 (m, 2H), 1.52 (d, J=12.0 Hz, 2H).

### Step C. 4-({8-Benzyl-2-oxo-3-[4-(trifluoromethoxy)phenyl]-1,3,8-triazaspiro[4.5]dec-1-yl}methyl)benzoic acid

A solution of 2.3 g methyl 4-({8-benzyl-2-oxo-3-[4-(trifluoromethoxy)phenyl]-1,3,8-triazaspiro-[4.5]dec-1-yl}methyl)benzoate in 40 mL methanol and 20 mL water was treated with 830 mg sodium hydroxide at 55° C. for 4 hours. After removing solvents, the residue was acidified with aq. HCl and the resulting precipitate collected by filtration to give the title compound as a white solid. ¹H NMR (CDCl₃, 500 MHz) 57.85 (d, J=8.5 Hz, 2H), 7.57˜7.59 (m, 4H), 7.53 (d, J=8.5 Hz, 2H), 7.43˜7.47 (m, 3H), 7.23 (d, J=8.5 Hz, 2H), 4.66 (s, 2H), 4.23 (s, 2H), 3.68 (br s, 4H), 3.01˜3.06 (m, 2H), 2.68˜2.76 (m, 2H), 1.65 (d, J=13.5 Hz, 2H). LC-MS: 1.88 min. (M+H=540.1).

### Step D. 4-({8-Benzyl-2-oxo-3-[4-(trifluoromethoxy)phenyl]-1,3,8-triazaspiro[4.5]dec-1-yl}methyl)-N-(1H-tetrazol-5-yl)benzamide

The title compound was prepared from 4-({8-benzyl-2-oxo-3-[4-(trifluoromethoxy)-phenyl]-1,3,8-triazaspiro[4.5]dec-1-yl}methyl)benzoic acid using the procedure in Step G of the previous Example. ¹H NMR (DMSO-d₆, 500 MHz) δ 8.07 (d, J=8.0 Hz, 2H), 7.62˜7.65 (m, 2H), 7.57 (d, J=8.0 Hz, 2H), 7.53 (d, J=8.5 Hz, 2H), 7.47˜7.49 (m, 2H), 7.41˜7.45 (m, 3H), 4.67 (s, 2H), 4.23 (br s, 2H), 3.31 (br s, 6H), 2.15˜2.25 (m, 4H). LC-MS: 1.87 min. (M+H=621.2).

### EXAMPLE 101

### N-[4-({8-BENZYL-2-OXO-3-[4-(TRIFLUOROMETHOXY)PHENYL]-1,3,8-TRIAZASPIRO[4.5]DEC-1-YL}METHYL)BENZOYL]-β-ALANINE

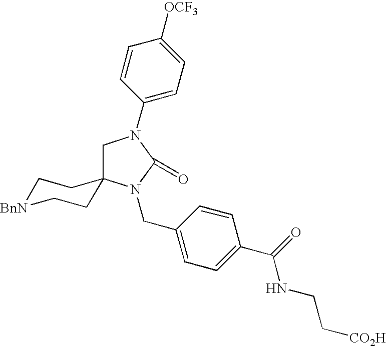

### Step A. Tert-butyl N-[4-({8-benzyl-2-oxo-3-[4-(trifluoromethoxy)phenyl]-1,3,8-triazaspiro[4.5]dec-1-yl}methyl)benzoyl]-β-alaninate

The title compound was prepared from the product of Step C Example 92 using procedure in Step A Example 4. ¹H NMR (CD₃OD, 500 MHz) δ 7.77 (d, J=8.0 Hz, 2H), 7.69˜7.72 (m, 2H), 7.43˜7.46 (m, 7H), 7.29 (d, J=8.5 Hz, 2H), 4.86 (s, 2H), 4.13 (br s, 2H), 3.96 (s, 2H), 3.58 (t, J=7.0 Hz, 2H), 3.33 (br s, 2H), 2.99 (br s, 2H), 2.55 (t, J=7.0 Hz, 2H), 2.08˜2.15 (m, 2H), 1.81 (br d, J=14 Hz, 2H). LC-MS: 2.08 min. (M+H=667.3).

### Step B. N-[4-({8-Benzyl-2-oxo-3-[4-(trifluoromethoxy)phenyl]-1,3,8-triazaspiro[4.5]dec-1-yl}methyl)benzoyl]-β-alanine

The title compound was prepared from tert-butyl N-[4-({8-benzyl-2-oxo-3-[4-(trifluoromethoxy)phenyl]-1,3,8-triazaspiro[4.5]dec-1-yl}methyl)benzoyl]-β-alaninate using procedure in Step B Example 4. ¹H NMR (DMSO-d₆, 500 MHz) δ 8.48 (t, J=5 Hz, 1H), 7.77 (d, J=8 Hz, 2H), 7.67˜7.73 (m, 2H), 7.35˜7.50 (m, 9H), 4.43 (s, 2H), 4.27 (s, 2H), 3.92 (s, 2H), 3.8 (br, 2H), 3.43 (dt, J=5 & 7 Hz, 2H), 3.15˜3.23 (m, 2H), 2.10˜2.17 (m, 2H), 1.72˜1.76 (m, 2H). LC-MS: 1.80 min. (M+H=611.2).

### EXAMPLE 102

### (2R)-3-{[4-({8-BENZYL-2-OXO-3-[4-(TRIFLUOROMETHOXY)PHENYL]-1,3,8-TRIAZASPIRO[4.5]DEC-1-YL}METHYL)BENZOYL]AMINO}-2-HYDROXYPROPANOIC ACID

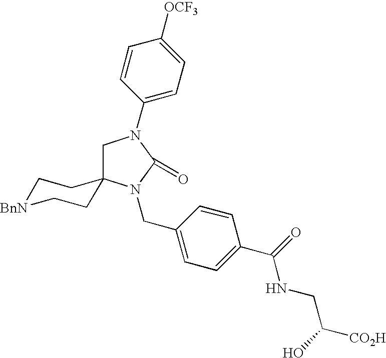

### Step A. Methyl (2R)-3-{[4-({8-benzyl-2-oxo-3-[4-(trifluoromethoxy)phenyl]-1,3,8-triazaspiro[4.5]dec-1-yl}methyl)benzoyl]amino}-2-hydroxypropanoate

The title compound was prepared from the product of Step C, Example 92 using procedure in Step D Example 10. ¹H NMR (CD₃OD, 500 MHz) δ 7.77 (d, J=8.5 Hz, 2H), 7.69˜7.72 (m, 2H), 7.47˜7.51 (m, 5H), 7.43 (d, J=8.0H, 2H), 7.29 (d, J=8.5 Hz, 2H), 4.53 (s, 2H), 4.36˜4.38 (m, 11H), 4.32 (s, 2H), 4.00 (s, 2H), 3.72 (s, 3H), 3.65˜3.71 (m, 2H), 3.49 (br d, J=12.5 Hz, 2H), 3.22˜3.31 (m, 2H), 2.18˜2.23 (m, 2H), 1.87 (d, J=14.0 Hz, 2H). LC-MS: 1.81 min. (M+H=641.2).

Step B. (2R)-3-{[4-({8-benzyl-2-oxo-3-[4-(trifluoromethoxy)phenyl]-1,3,8-triazaspiro[4.5]dec-1-yl}methyl)benzoyl]amino}-2-hydroxypropanoic acid The title compound was prepared from the product of the previous step using procedure in Example 11. ¹H NMR (CD₃OD, 500 MHz) δ 7.78 (d, J=8.5 Hz, 2H), 7.69˜7.72 (m, 2H), 7.42˜7.51 (m, 7H), 7.28 (m, J=9 Hz, 2H), 4.54 (s, 2H), 4.35˜4.37 (m, 1H), 4.31 (s, 2H), 4.00 (s, 2H), 3.76 (br d, J=13 Hz, 1H), 3.63 (dd, J=7 & 13 Hz, 1H), 3.46 (br d, J=12 Hz, 2H), 3.22˜3.27 (m, 2H), 2.23˜2.30 (m, 2H), 1.83 (d, J=14 Hz, 2H). LC-MS: 2.75 min. (M+H=627.4).

### EXAMPLE 103

### CIS/TRANS-4-({9-TERT-BUTYL-3-OXO-4-[4-(TRIFLUOROMETHOXY)PHENYL]-2,4-DIAZASPIRO[5.5]UNDEC-2-YL}METHYL)-N-(1H-TETRAZOL-5-YL)BENZAMIDE

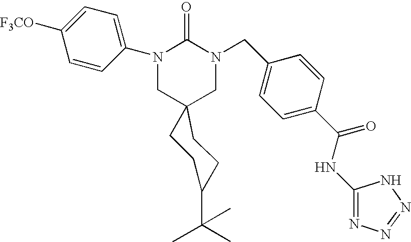

### Step A: Methyl 4-tert-butylcyclohexanecarboxylate

To a solution of 5 g (27.1 mmol) of 4-tert-butylcyclohexanecarboxylic acid in 100 mL of dichloromethane was added 16.3 mL (32.6 mmol) of a 2M solution of oxalyl chloride in dichloromethane, followed by 100 mL of N,N-dimethylformamide. The resultant mixture was stirred at ambient temperature for 3 hours, concentrated in vacuo and 100 mL of methanol added. After concentration in vacuo the residue was filtered through a short silica gel plug, eluting with 10% ethyl acetate/hexane to give the title compound as a mixture of cis and trans isomers. HPLC/MS: calcd for (C12H22O2) 198, found 199 (M+H).

### Step B: Dimethyl 4-tert-butylcyclohexane-1,1-dicarboxylate

To a solution of 2.17 mL (15.5 mmol) of diethylamine in 15 mL of tetrahydrofuran at −20 C was added 9.7 mL (15.5 mmol) of n-butyl lithium as a 1.6 M solution in hexanes. The resultant mixture was stirred at 0° C. for 45 minutes then was cooled to −20° C. and a solution of 2.56 g (12.9 mmol) of the product from Step A in 10 mL of tetrahydrofuran was added. After stirring for 1.5 hours at −15 to −20° C., 1.5 mL (19.4 mmol) of methyl chloroformate was added and the reaction mixture warmed to ambient temperature. The mixture was diluted with ethyl acetate and the organic layer washed sequentially with one portion of water and one portion of brine. The organic layer was dried over magnesium sulfate, concentrated in vacuo and the residue purified by column chromatography (silica gel, 2.5% ethyl acetate/hexane to 20% ethyl acetate hexane) to give the title compound. ¹H NMR (500 MHz, CDCl₃) & 3.76 (s, 3H), 3.70 (s, 3H), 2.42 (d, J=13 Hz, 2H), 1.76-1.58 (m, 4H), 1.15-0.98 (m, 3H), 0.83 (s, 9H).

### Step, C: 4-tert-Butyl-1-(hydroxymethyl)cyclohexylmethanol

To a suspension of 660 mg (16.5 mmol) of lithium aluminum hydride in 10 mL of tetrahydrofuran at 0° C. was added a solution of 2.12 g (8.25 mmol) of the product from step B in 20 mL of tetrahydrofuran. The resultant mixture was stirred at ambient temperature for 16 hours. The reaction was quenched by sequential addition of 0.66 mL water, 0.66 mL 2N aqueous sodium hydroxide solution, and 1.98 mL water. The mixture was filtered through celite, the filter pad washed well with tetrahydrofuran, and the filtrate concentrated in vacuo. The residue was purified by column chromatography (silica gel, 40% ethyl acetate hexane) to give the title compound. ¹H NMR (500 MHz, CDCl₃) δ 3.76 (s, 2H), 3.52 (s, 2H), 2.0 (broad s, 2H), 1.81 (d, J=12.2 Hz, 2H), 1.66-1.61 (m, 2H), 1.17-0.97 (m, 5H), 0.88 (s, 9H).

### Step D: Methyl 4-{[{[4-tert-butyl-1-(hydroxymethyl)cyclohexyl]methyl}({[4-(trifluoromethoxy)phenyl]amino}carbonyl)amino]methyl}benzoate

To a solution of 300 mg (1.5 mmol) of the product from Step C in 6 mL of dichloromethane was added 284 mg (0.67 mmol) of Dess-Martin periodinane. The resultant mixture was stirred at ambient temperature for 2.5 hours, filtered through celite, and concentrated in vacuo. This unpurified aldehyde was added to a solution of 132 mg (0.80 mmol) methyl 4-(aminomethyl)-benzoate and 227 mg (1.07 mmol) of sodium triacetoxyborohydride in 6 mL of 1,2-dichloroethane. The resultant mixture was stirred at ambient temperature for 16 hours, diluted with dichloromethane and washed sequentially with one portion of saturated aqueous sodium bicarbonate solution, one portion of water, and one portion of brine. The organic layer was dried over magnesium sulfate, concentrated in vacuo and added to a solution of 163 mg (0.80 mmol) of 4-(trifluoromethoxy)phenyl isocyanate in 6 mL of chloroform. After stirring at ambient temperature for 4 hours, the reaction mixture was concentrated in vacuo and purified by column chromatography (silica gel, 20% ethyl acetate/hexane) to provide the title compound as a mixture of isomers. HPLC/MS: calcd for (C29H37F3N2O5) 550, found 551 (M+H).

### Step E: Cis and trans Methyl 4-({9-tert-butyl-3-oxo-4-[4-(trifluoromethoxy)phenyl]-2,4-diazaspiro[5.5]undec-2-yl}methyl)benzoate

To a solution of 132 mg (0.24 mmol) of the product from Step D in 5 mL of tetrahydrofuran was added 126 mg (0.48 mmol) of triphenylphosphine. A solution of 76 μL (0.48 mmol) of diethyl azodicarboxylate in 1.5 mL of tetrahydrofuran was added dropwise and the resultant mixture stirred at ambient temperature for 1 hour. The mixture was diluted with ethyl acetate and the organic layer washed sequentially with one portion each of water and brine. The organic layer was dried over magnesium sulfate, concentrated in vacuo, and the residue purified by preparative TLC (silica gel, 30% ethyl acetate/hexane) to provide both isomers of the title compound. HPLC/MS: calcd for (C29H35F3N2O4) 532, found 533 (M+H). ¹H NMR (500 MHz, CDCl₃) Cis isomer: δ 8.04 (d, J=8.2 Hz, 2H), 7.51 (d, J=8 Hz, 2H), 7.41 (d, J=8.9 Hz, 2H), 7.30 (d, J=8.5 Hz, 2H), 4.66 (s, 2H), 3.93 (s, 3H), 3.40 (s, 2H), 3.23 (s, 2H), 1.77 (d, J=16.7 Hz, 2H), 1.52-150 (m, 2H), 1.27-1.18 (m, 2H), 1.01-0.95 (m, 1H), 0.76 (s, 9H), 0.76-0.65 (m, 2H). Trans isomer: δ 8.02 (d, J=8.2 Hz, 2H), 7.46 (d, J=8.3 Hz, 2H), 7.42-7.40 (m, 2H), 7.32 (d, J=8.4 Hz, 2H), 4.64 (s, 2H), 3.92 (s, 3H), 3.61 (s, 2H), 3.06 (s, 2H), 1.90-1.84 (m, 2H), 1.73-1.66 (m, 2H), 1.24-1.18 (m, 2H), 1.13-1.02 (m, 3H), 0.84 (s, 9H).

### Step F: Cis and trans 4-({9-tert-butyl-3-oxo-4-[4-(trifluoromethoxy)phenyl]-2,4-diazaspiro[5.5]undec-2-yl}methyl)benzoic acids

To a solution of 51 mg (0.096 mmol) of the cis isomer from step E in 2 mL of dioxane and 1 mL of water was added 20 mg (0.48 mmol) of lithium hydroxide monohydrate. The reaction mixture was heated to 40° C. for 2 hours then concentrated in vacuo. The residue was suspended in water, acidified with concentrated HCl and extracted into ethyl acetate. The organic layer was dried over magnesium sulfate and concentrated in vacuo to give the title compound. HPLC/MS: calcd for (C28H33F3N2O4) 518, found 519 (M+H). In a similar manner the trans isomer was converted to the carboxylic acid.

### Step G: Trans-4-({9-tert-Butyl-3-oxo-4-[4-(trifluoromethoxy)phenyl]-2,4-diazaspiro-[5.5]undec-2-yl}methyl)-N-(1H-tetraazol-5-yl)benzamide

To a solution of 21.1 mg (0.041 mmol) of the product from Step F in 2 mL of dimethylformamide was added 5.5 mg (0.053 mmol) of 5-aminotetrazole monohydrate, 21 μL (0.123 mmol) of N,N-diisopropylethylamine, and 24.7 mg (0.053 mmol) of bromo-tris-pyrrolidino-phosphonium hexafluorophosphate (PyBroP). After stirring at ambient temperature for 16 hours, the mixture was diluted with ethyl acetate and the organic layer washed with three portions of 1 N aq. HCl. The organic layer was dried over magnesium sulfate, concentrated in vacuo, and the residue purified by reverse phase preparative HPLC using a gradient elution of acetonitrile-water containing 0.1% trifluoroacetic acid to give the title compound after lyophilization. HPLC/MS: calcd for (C29H34F3N7O3) 585, found 586 (M+H). ¹H NMR (500 MHz, CD₃OD) δ 8.06 (d, J=8.2 Hz, 2H), 7.55 (d, J=8.2 Hz, 2H), 7.42 (d, J=8.9 Hz, 2H), 7.30 (d, J=8.7 Hz, 2H), 4.69 (s, 2H), 3.42 (s, 2H), 3.28, (s, 2H), 1.81 (d, J=13 Hz, 2H), 1.6-1.52 (m, 2H), 1.32-1.20 (m, 2H), 1.02-0.93 (m, 1H), 0.82-0.75 (m, 2H), 0.78 (s, 9H).

### Cis-4-({9-ten-Butyl-3-oxo-4-[4-(trifluoromethoxy)phenyl]-2,4-diazaspiro[5.5]undec-2-yl}methyl)-N-(1H-tetraazol-5-yl)benzamide

The product from Step F was converted to the title compound using the procedure outlined above. HPLC/MS: calcd for (C29H34F3N7O3) 585, found 586 (M+H). ¹H NMR (500 MHz, CD₃OD) δ 8.05 (d, J=8.2 Hz, 2H), 7.54 (d, J=8.1 Hz, 2H), 7.42 (d, J=8.9 Hz, 2H), 7.33 (d, J=8.6 Hz, 2H), 4.67 (s, 2H), 3.09 (s, 2H), 1.90 (d, J=14.4 Hz, 2H), 1.72-1.66 (m, 2H), 1.26-1.18 (m, 2H), 1.17-1.01 (m 3H), 0.84 (s, 9H).

### EXAMPLE 104

### CIS-N-[4-({-TERT-BUTYL-3-OXO-4-[4-(TRIFLUOROETHOXY)PHENYL]-2,4-DIAZASPIRO[5.5]-UNDEC-2-YL}METHYL)BENZOYL]-β-ALANINE

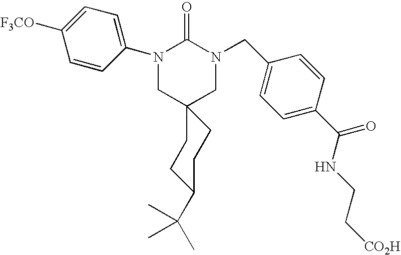

To a solution of 25.0 mg (0.048 mmol) of the Cis product from Example 103, step F in 2 mL of dimethylformamide was added 12.0 mg (0.066 mmol) of β-alanine t-butyl ester hydrochloride, 10.0 mg (0.074 mmol) of 1-hydoxybenzotriazole (HOBT), 27 mL (0.16 mmol) of N,N-diisopropylethylamine, and 12.5 mg (0.065 mmol) of 1-[3-(Dimethylamino)propyl]-2-ethylcarbodiimide hydrochloride (EDC). After stirring at ambient temperature for 16 hours, the mixture was diluted with ethyl acetate and the organic layer washed sequentially with one portion of saturated aqueous sodium bicarbonate solution, three portions of water, and one portion of brine. The organic layer was dried over magnesium sulfate and concentrated in vacuo. The residue was treated with 2 mL each of dichloromethane and trifluoroacetic acid at ambient temperature for 2 hours. The mixture was concentrated in vacuo and purified by reverse phase preparative HPLC using a gradient elution of acetonitrile-water containing 0.1% trifluoroacetic acid to give the title compound after lyophilization. HPLC/MS: calcd for (C31H38F3N3O5) 589, found 590 (M+H). ¹H NMR (500 MHz, CD₃OD) δ 7.80 (d, J=8.2 Hz, 2H), 7.43-7.39 (m, 4H), 7.31 (d, J=8.5 Hz, 211), 4.61 (s, 2H), 3.63 (t, J=6.9 Hz, 2H), 3.59 (s, 2H), 3.04 (s, 2H), 2.65 (t, J=6.9 Hz, 2H), 1.85 (d, J=13.5 Hz, 2H), 1.71-1.64 (m, 2H), 1.22-1.15 (m, 2H), 1.10-0.99 (m, 3H), 0.83 (s, 9H).

### EXAMPLE 105

### CIS-(2R)-3-{[4-({9-TERT-BUTYL-3-OXO-4-[4-(TRIFLUOROMETHOXY)PHENYL]-2,4-DIAZASPIRO[5.5]-UNDEC-2-YL}METHYL)BENZOYL]AMINO}-2-HYDROXYPROPANOIC ACID

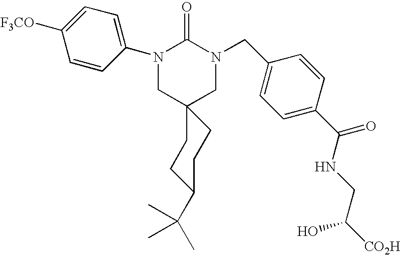

To a solution of 25.0 mg (0.048 mmol) of the cis product from Example 103, Step F in 2 mL of dimethylformamide was added 7.0 mg (0.053 mmol) of methyl (2R)-3-amino-2-hydroxypropanoate, 10.0 mg (0.074 mmol) of 1-hydoxybenzotriazole (HOBT), 27 μL (0.16 mmol) of N,N-diisopropylethylamine, and 12.5 mg (0.065 mmol) of 1-[3-(Dimethylamino)propyl]-2-ethylcarbodiimide hydrochloride (EDC). After stirring at ambient temperature for 16 hours, the mixture was diluted with ethyl acetate and the organic layer washed sequentially with one portion of saturated aqueous sodium bicarbonate solution, three portions of water, and one portion of brine. The organic layer was dried over magnesium sulfate, concentrated, and the residue purified by preparative TLC (silica gel, 50% ethyl acetate/hexane) to give the title compound as its methyl ester. HPLC/MS: calcd for (C32H40F3N3O6) 619, found 620 (M+H). ¹H NMR (500 MHz, CD₃OD) δ 7.82 (d, J=8.3 Hz, 2H), 7.44 (d, J=8 Hz, 2H), 7.41 (d, J=8.9 Hz, 2H), 7.32 (d, J=8.7 Hz, 2H), 4.63 (s, 2H), 4.41 (t, J=5.6 Hz, 1H), 3.77 (s, 3H), 3.77-3.65 (m, 2H), 3.60 (s, 2H), 3.05 (s, 2H), 1.87 (d, J=13.5 Hz, 2H), 1.72-1.63 (m, 2H), 1.23-1.16 (m, 2H), 1.13-0.98 (m, 3H), 0.84 (s, 9H).

To 15.9 mg (0.025 mmol) of the methyl ester in 1 mL each of tetrahydrofuran and water, was added 6 mg (0.14 mmol) of lithium hydroxide hydrate and the resultant mixture was stirred at ambient temperature for 16 hours. The tetrahydrofuran was removed in vacuo and the aqueous residue acidified to pH 2 with concentrated hydrochloric acid. The aqueous layer was extracted with three portions of ethyl acetate and the organic layer dried over magnesium sulfate and concentrated in vacuo. Purification by reverse phase preparative HPLC using a gradient elution of acetonitrile-water containing 0.1% trifluoroacetic acid provided the title compound after lyophilization. HPLC/MS: calcd for (C31H38F3N3O6) 605, found 606 (M+H). ¹H NMR (500 MHz, CD₃OD) δ 7.83 (d, J=8.3 Hz, 2H), 7.43 (d, J=8.4 Hz, 2H), 7.39 (d, J=8.9 Hz, 2H), 7.31 (d, J=8.5 Hz, 2H), 4.61 (s, 2H), 4.39-4.37 (m, 1H), 3.81-3.62 (m, 2H), 3.59 (s, 2H), 3.04 (s, 2H), 1.86 (d, J=13.5 Hz, 2H), 1.70-1.63 (m, 2H), 1.21 (m, 2H), 1.10-1.00 (m, 3H), 0.83 (s, 9H).

Biological Assays

The ability of the compounds of the present invention to inhibit the binding of glucagon and their utility in treating or preventing type 2 diabetes mellitus and the related conditions can be demonstrated by the following in vitro assays.

Glucagon Receptor Binding Assay

A stable CHO (Chinese hamster ovary) cell line expressing cloned human glucagon receptor was maintained as described (Chicchi et al. J Biol Chem 272, 7765-9(1997); Cascieri et al. J Biol Chem 274, 8694-7(1999)). To determine antagonistic binding affinity of compounds 0.002 mg of cell membranes from these cells were incubated with ¹²⁵I-Glucagon (New England Nuclear, MA) in a buffer containing 50 mM Tris-HCl (pH 7.5), 5 mM MgCl₂, 2 mM EDTA, 12% Glycerol, and 0.200 mg WGA coated PVT SPA beads (Amersham), +/−compounds or 0.001 mM unlabeled glucagon. After 4-12 hours incubation at room temperature, the radioactivity bound to the cell membranes was determined in a radioactive emission detection counter (Wallac-Microbeta). Data was analyzed using the software program Prism® from GraphPad. The IC₅₀ were calculated using non-linear regression analysis assuming single site competition.

Inhibition of Glucagon-Stimulated Intracellular cAMP Formation

Exponentially growing CHO cells expressing human glucagon receptor were harvested with the aid of enzyme-free dissociation media (Specialty Media), pelleted at low speed, and re-suspended in cell suspension buffer [75 mM Tris-HCl pH7.5, 250 mM Sucrose, 25 mM MgCl₂, 1.5 mM EDTA, 0.1 mM Ro-20-1724 (Biomol, Inc.), 0.2% bovine serum albumin and one tablet of complete™ (Boehringer), which contains a cocktail of protease inhibitors, for each 50 ml of buffer]. An adenylate cyclase assay was setup using an Adenylate Cyclase Assay kit (SMP-004B) from New England Nuclear (NEN) as per manufacturer instructions. Briefly, compounds were diluted from stocks in a cell stimulation buffer supplied with the kit. Cells prepared as above were preincubated in flash plates coated with anti-cAMP antibodies (NEN) in presence of compounds or DMSO controls for 40 minutes, and then stimulated with glucagon (250 μM) for an additional 40 minutes. The cell stimulation was stopped by addition of equal amount of a detection buffer containing lysis buffer as well as ¹²⁵I-labeled cAMP tracer (NEN). After 3-6 h of incubation at room temperature the bound radioactivity was determined in a liquid scintillation counter TopCount-Packard Instruments). Activity of test compounds was calculated by comparing to the total scintillation signal (CPM) of control samples with no compound and with 0.001 mM unlabeled-glucagon.

Certain embodiments of the invention has been described in detail; however, numerous other embodiments are contemplated as falling within the invention.

What is claimed is:

1. A compound represented by formula I: 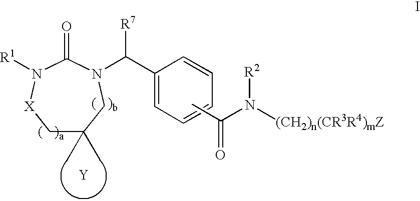
or a pharmaceutically acceptable salt or solvate thereof, wherein:
a and b are independently selected from the integers 0 and 1, such that the sum of a and b is 0 or 1;
X is selected from CH₂ and C(O);
R¹ is selected from the group consisting of:
(1) C₁₋₁₅ alkyl optionally substituted with up to five groups as follows: (a) 1-3 OH groups; (b) 1 oxo group; (c) 1-5 halo groups, up to a perhaloalkyl group; (d) 1-3 C₁₋₆ alkoxy groups optionally substituted with up to five halo or a perhaloalkoxy, or up to 2 hydroxy or CO₂R⁶ groups; (e) 1-2 CO₂R⁶ groups and (f) 1-2 phenyl groups, each optionally substituted as follows: 1-5 halo groups, (2) 1-2 OH, CO₂R⁶, CN or S(O)ₚR⁵ groups, and (3) 1-2 C₁₋₆ alkyl or alkoxy groups, each optionally substituted with 1-5 halo, up to perhaloalkyl, and 1-2 OH or CO₂R⁶ groups; and
(2) aryl or heteroaryl, optionally substituted as set forth below:
(a) 1-3 hydroxy groups; (b) 1-5 halo groups; (c) 1-3 C₁₋₁₅ alkyl or alkoxy groups, each optionally substituted with up to five halo and 1-2 hydroxy or CO₂R⁶ groups; (d) 1-2 CO₂R⁶, CN, S(O)ₚR⁵ or CONR⁹R¹⁰ groups; (e) NR⁹R¹⁰; (f) SCF₃; (g) phenyl, heteroaryl or O-phenyl, said group being optionally substituted with 1-5 halo groups, 1-2 OH, CO₂R⁶, CN or S(O)ₙR⁵ groups, and 1-2 C₁₋₆ alkyl or alkoxy groups, each optionally substituted with 1-5 halo, up to perhaloalkyl, and 1-2 OH or CO₂R⁶ groups;
R² represents H or C₁₋₆alkyl;
R³ represents H or F;
R⁴ is selected from the group consisting of H, F and OH;
or R³ and R⁴ are taken in combination and represent an oxo group;
R⁵ represents a C₁₋₁₀alkyl group;
R⁶ represents H or C₁₋₁₀alkyl, optionally substituted with OH, OC₁₋₆alkyl, CO₂H, CO₂C₁₋₆alkyl, and 1-3 halo groups;
R⁷ represents H, CO₂R⁶, C₁₋₆alkyl optionally substituted with OH, OC₁₋₆alkyl, CO₂R⁶ or 1-3 halo groups;
R⁸ and R⁹ are independently selected from H and C₁₋₆alkyl;
R¹⁰ is H or is independently selected from:
(a) C₁₋₁₀alkyl, optionally substituted with OH, OC₁₋₆alkyl, CO₂H, CO₂C₁₋₆alkyl, and 1-3 halo groups; (b) aryl or C₁₋₆ alkaryl, each optionally substituted with 1-5 halos and 1-3 members selected from the group consisting of: CN, OH, C₁₋₁₀alkyl and OC₁₋₁₀ alkyl, said alkyl and alkoxy being further optionally substituted with 1-5 halo groups up to perhalo; (c) heterocycle, or C₁₋₆alkyl-heterocycle, optionally substituted with 1-5 halo groups and 1-3 groups selected from: oxo, C₁₋₁₀alkyl and OC₁₋₁₀alkyl, said alkyl and alkoxy being further optionally substituted with 1-5 halo groups up to perhalo; and (d) heteroaryl or C₁₋₆alkyl-heteroaryl, optionally substituted with 1-5 halo groups and 1-3 groups selected from: C₁₋₁₀alkyl and OC₁₋₁₀ alkyl, said alkyl and alkoxy being further optionally substituted with 1-5 halo groups up to perhalo;
R¹¹ is independently selected from the group consisting of:
(a) C₁₋₁₀alkyl, optionally substituted with OH, OC₁₋₆alkyl, CO₂H, CO₂C₁₋₆alkyl, and 1-3 halo groups; (b) aryl or C₁₋₆ alkaryl, each optionally substituted with 1-5 halos and 1-3 members selected from the group consisting of: CN, OH, C₁₋₁₀alkyl and OC₁₋₁₀ alkyl, said alkyl and alkoxy being further optionally substituted with 1-5 halo groups up to perhalo; (c) heterocycle, or C-alkyl-heterocycle, optionally substituted with 1-5 halo groups and 1-3 groups selected from: oxo, C₁₋₁₀alkyl and OC₁₋₁₀ alkyl, said alkyl and alkoxy being further optionally substituted with 1-5 halo groups up to perhalo; and (d) heteroaryl or C₁₋₆alkyl-heteroaryl, optionally substituted with 1-5 halo groups and 1-3 groups selected from: C₁₋₁₀alkyl and OC₁₋₁₀ alkyl, said alkyl and alkoxy being further optionally substituted with 1-5 halo groups up to perhalo;
Y represents a 4 to 8 membered spirocarbocyclic ring or a spiroheterocyclic ring containing up to three heteroatoms, 0-1 of which are selected from O and S and 0-3 of which are N,
said spirocarbocyclic or spiroheterocyclic ring being optionally substituted on either carbon or nitrogen atoms with up to three groups independently selected as follows:
(a) 1-2 phenyl groups, each being optionally substituted with one to five groups independently selected from the group consisting of: (1) 1-3 hydroxy groups; (2) 1-5 halo groups; (3) 1-3 C₁₋₈alkyl or alkoxy groups, each being further optionally substituted with 1-5 halo or 1-2 OH or CO₂R⁶ groups, and (4) 1-2 CO₂R⁶, CN, S(O)ₚR⁵, CONR⁹R¹⁰ or NO₂ groups;
(b) C₁₋₁₀ alkyl optionally substituted with 1-5 groups selected as follows: (i) 1-3 hydroxy groups; (ii) 1 oxo group; (iii) 1-5 halo groups up to perhalo; (iv) 1-3 C₁₋₁₀ alkoxy groups, optionally substituted with 1-5 halo groups up to perhalo, or 1-2 hydroxy or CO₂R⁶ groups; (v) 1-2 CO₂R⁶ groups; (vi) phenyl, optionally substituted with one to five groups independently selected from the group consisting of: (a) 1-3 hydroxy groups; (b) 1-5 halo groups; (c) 1-3 C₁₋₆ alkyl or alkoxy groups, optionally substituted with 1-5 halo groups up to perhalo, or 1-2 hydroxy or CO₂R⁶ groups; (d) 1-2 CO₂R⁶, CN, S(O)ₚR⁵, CONR⁹R¹⁰ or NO₂ groups; (e) 1-2 phenyl rings, each of which is optionally substituted as follows: 1-3 C₋₁₀ alkyl or alkoxy groups, each being further optionally substituted with 1-5 halo up to perhalo, or 1-2 hydroxy or CO₂R⁶ groups;
said spirocarbocyclic or spiroheterocyclic ring being further optionally substituted on a carbon atom with a member selected from the group consisting of:
(a) —NR⁸—C(O)—NR⁹R¹⁰; (b) —NR⁸—CO₂R¹¹; (c) —NR⁸—C(O)R¹¹; (d) —NR⁹R¹⁰;
(e) —NR⁸SO₂R¹¹; (f) —SO₂—NR⁹R¹⁰; (g) —C(O)NR⁹R¹⁰ and (h) —OC(O)—NR⁹R¹⁰;
and when said ring contains a nitrogen atom, said ring being further optionally substituted on the nitrogen atom with a member selected from the group consisting of:
(a) —C(O)NR⁹R¹⁰; (b) —CO₂R¹¹; (c) C(O)R¹¹; and (d) —SO₂R¹¹;
m and p are independently selected from 0, 1 and 2, and n is an integer from 0 to 6,
when both m and n are zero, Z is selected from 5-tetrazolyl and 5-(2-oxo-1,3,4-oxadiazolyl) and when one of m and n is other than zero, Z is selected from the group consisting of: CO₂R⁶, with R⁶ as defined above, 5-tetrazolyl and 5-(2-oxo-1,3,4-oxadiazolyl).

2. A compound in accordance with claim 1 wherein:
R¹ is selected from the group consisting of:
(1) C₁₋₆ alkyl optionally substituted with 1-3 groups selected from: OH, halo, C₁₋₃ alkoxy, halo-C₁₋₃alkoxy and phenyl, said phenyl being optionally substituted with 1-3 halo groups, SO₂R⁵, and 1-2 C₁₋₃alkyl or alkoxy groups optionally substituted with 1-3 halo groups, and
(2) aryl optionally substituted with 1-3 halo groups; 1-2 C₁₋₃alkyl or alkoxy groups, each optionally substituted with 1-3 halo groups; —NR⁹R¹⁰ wherein R⁹ and R¹⁰ are H or methyl; SCF₃ and heteroaryl.

3. A compound in accordance with claim 2 wherein:
R¹ represents phenyl optionally substituted with 1-2 groups selected from Br, Cl; trifluoromethyl and trifluoromethoxy.

4. A compound in accordance with claim 1 wherein: X represents CH₂.

5. A compound in accordance with claim 1 wherein a and b represent 0 or a represents 1 and b represents 0.

6. A compound in accordance with claim 1 wherein:
Y represents a spiroC₄₋₈cycloalkyl group or a 5-6 membered spiroheterocyclic group containing 1 N atom,
said ring being optionally substituted with a C₁₋₆ alkyl group, which is optionally substituted with 1-3 halo groups or 1 Phenyl ring that is optionally substituted with 1-2 halo, 1-2 C₁₋₃ alkyl or alkoxy groups, said alkyl and alkoxy substituents being further optionally substituted with 1-3 halo groups.

7. A compound in accordance with claim 6 wherein:
Y represents a spirocyclohexyl or spiropiperidinyl group that is substituted with a C₁₋₄ alkyl group that is optionally substituted with a phenyl ring.

8. A compound in accordance with claim 7 wherein:
Y represents a spirocyclohexyl group substituted with a t-butyl group at the 4 position.

9. A compound in accordance with claim 1 wherein: R² is H or C₁₋₃alkyl.

10. A compound in accordance with claim 9 wherein: R² represents H.

11. A compound in accordance with claim 1 wherein: R⁷ represents H or methyl.

12. A compound in accordance with claim 11 wherein R⁷ represents H.

13. A compound in accordance with claim 1 wherein:
n and m represent 0, and Z represents a 5-tetrazolyl group.

14. A compound in accordance with claim 1 wherein:
m represents 0, n represents 2, and Z represents a CO₂R⁶ group.

15. A compound in accordance with claim 1 wherein:
m and n each represent 1, R³ represents OH, R⁴ represents H and Z represents a CO₂R⁶ group.

16. A compound in accordance with claim 1 wherein:
R¹ is selected from the group consisting of:
(1) C₁₋₆ alkyl optionally substituted with 1-3 groups selected from: OH, halo, C₁₋₃ alkoxy, halo-C₁₋₃alkoxy and phenyl, said phenyl being optionally substituted with 1-3 halo groups, SO₂R⁵, and 1-2 C₁₋₃alkyl or alkoxy groups optionally substituted with 1-3 halo groups, and
(2) aryl optionally substituted with 1-3 halo groups; 1-2 C₁₋₃alkyl or alkoxy groups, each optionally substituted with 1-3 halo groups; —NR⁹R¹⁰ wherein R⁹ and R¹⁰ are H or methyl; SCF₃ and heteroaryl;
X represents CH₂;
a and b represent 0 or a represents 1 and b represents 0;
Y represents a spiroC₄₋₈cycloalkyl group or a 5-6 membered spiroheterocyclic group containing 1 N atom,
said ring being optionally substituted with a C₁₋₆ alkyl group, which is optionally substituted with 1-3 halo groups or 1 Phenyl ring that is optionally substituted with 1-2 halo, 1-2 C₁₋₃ alkyl or alkoxy groups, said alkyl and alkoxy substituents being further optionally substituted with 1-3 halo groups;
R² is H or C₁₋₃alkyl;
R⁷ represents H or methyl;
m and n represent 0, and Z represents a 5-tetrazolyl group.

17. A compound in accordance with claim 1 wherein:
R¹ is selected from the group consisting of:
(1) C₁₋₆ alkyl optionally substituted with 1-3 groups selected from: OH, halo, C₁₋₃ alkoxy, halo-C₁₋₃alkoxy and phenyl, said phenyl being optionally substituted with 1-3 halo groups, SO₂R⁵, and 1-2 C₁₋₃alkyl or alkoxy groups optionally substituted with 1-3 halo groups, and
(2) aryl optionally substituted with 1-3 halo groups; 1-2 C₁₋₃alkyl or alkoxy groups, each optionally substituted with 1-3 halo groups; —NR⁹R¹⁰ wherein R⁹ and R¹⁰ are H or methyl; SCF₃ and heteroaryl;
X represents CH₂;
a and b represent 0 or a represents 1 and b represents 0;
Y represents a spiroC₄₋₈cycloalkyl group or a 5-6 membered spiroheterocyclic group containing 1 N atom,
said ring being optionally substituted with a C₁₋₆ alkyl group, which is optionally substituted with 1-3 halo groups or 1 Phenyl ring that is optionally substituted with 1-2 halo, 1-2 C₁₋₃ alkyl or alkoxy groups, said alkyl and alkoxy substituents being further optionally substituted with 1-3 halo groups;
R² is H or C₁₋₃alkyl;
R⁷ represents H or methyl;
m represents 0, n represents 2, and Z represents a CO₂R⁶ group.

18. A compound in accordance with claim 1 wherein:
R¹ is selected from the group consisting of:
(1) C₁₋₆ alkyl optionally substituted with 1-3 groups selected from: OH, halo, C₁₋₃ alkoxy, halo-C₁₋₃alkoxy and phenyl, said phenyl being optionally substituted with 1-3 halo groups, SO₂R⁵, and 1-2 C₁₋₃alkyl or alkoxy groups optionally substituted with 1-3 halo groups, and
(2) aryl optionally substituted with 1-3 halo groups; 1-2 C₁₋₃alkyl or alkoxy groups, each optionally substituted with 1-3 halo groups; —NR⁹R¹⁰ wherein R⁹ and R¹⁰ are H or methyl; SCF₃ and heteroaryl;
X represents CH₂;
a and b represent 0 or a represents 1 and b represents 0;
Y represents a spiroC₄₋₈cycloalkyl group or a 5-6 membered spiroheterocyclic group containing 1 N atom,
said ring being optionally substituted with a C₁₋₆ alkyl group, which is optionally substituted with 1-3 halo groups or 1 Phenyl ring that is optionally substituted with 1-2 halo, 1-2 C₁₋₃ alkyl or alkoxy groups, said alkyl and alkoxy substituents being further optionally substituted with 1-3 halo groups;
R² is H or C₁₋₃alkyl;
R⁷ represents H or methyl;
m and n each represent 1, R³ represents OH, R⁴ represents H and Z represents a CO₂R⁶ group.

19. A compound in accordance with claim 1 selected from the following table:
TABLE 1

| Compound | Compound |
|---|---|
| 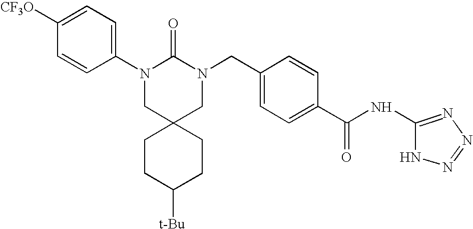 | 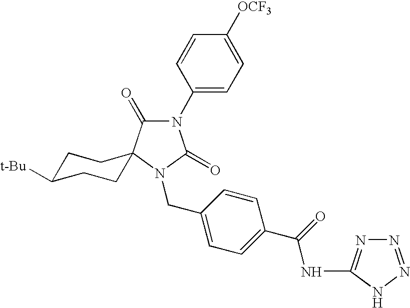 |
| 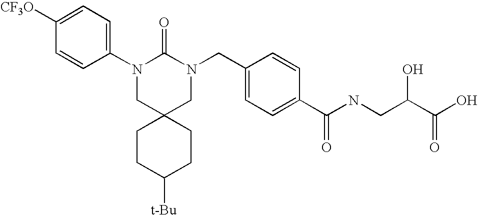 | 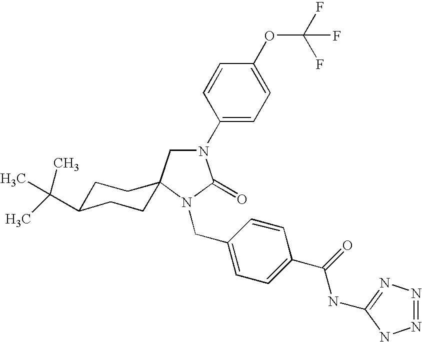 |
| 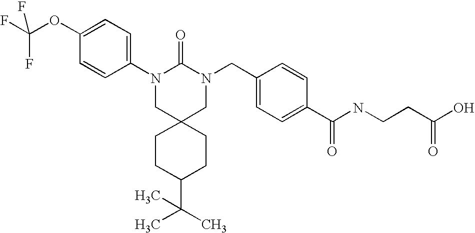 | 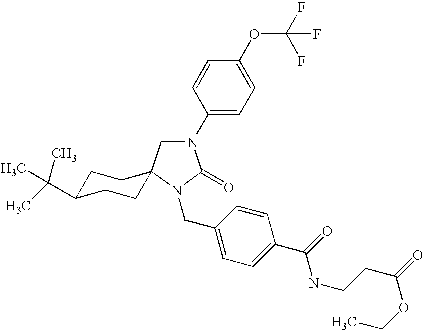 |
| 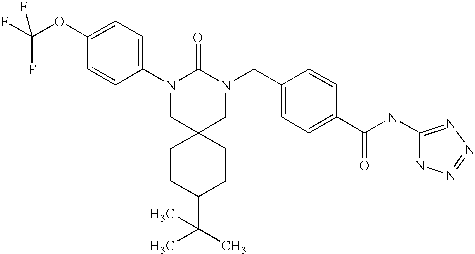 | 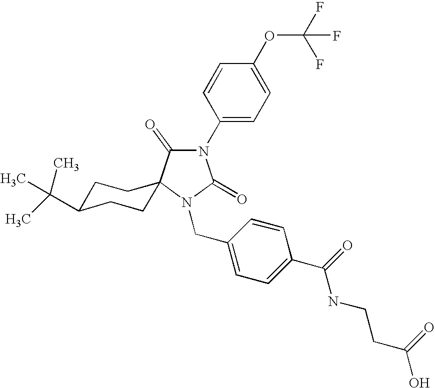 |
| 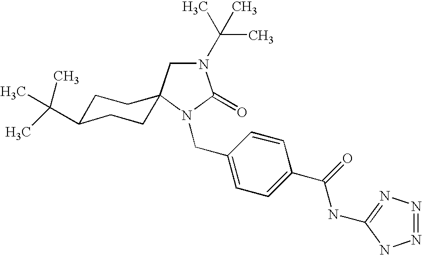 | 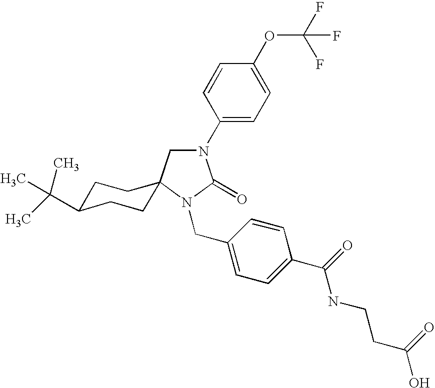 |
| 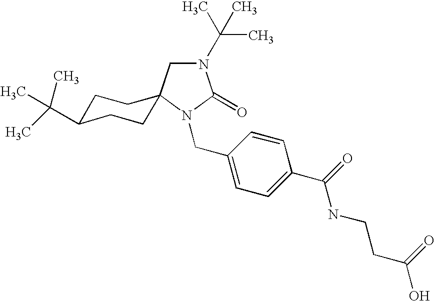 | 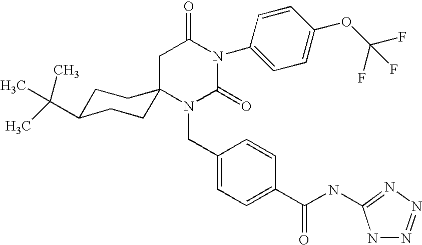 |
| 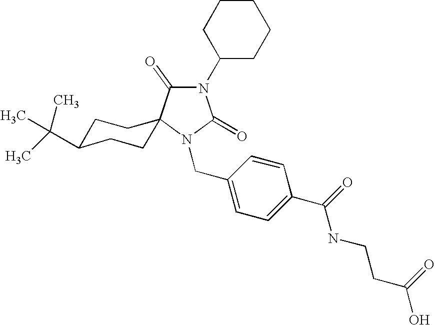 | 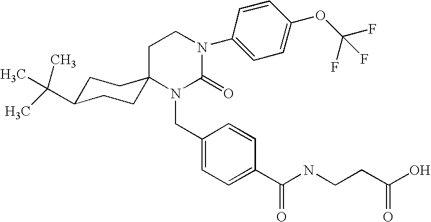 |
| 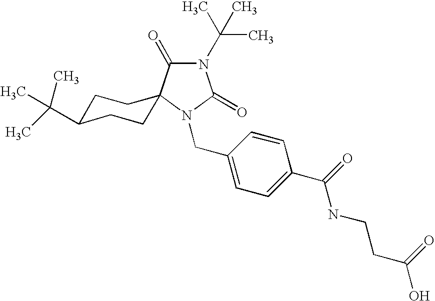 | 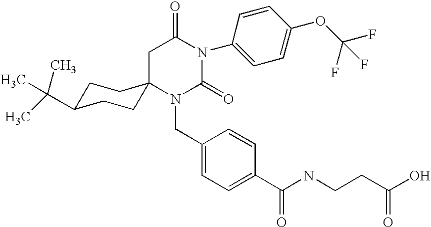 |
| 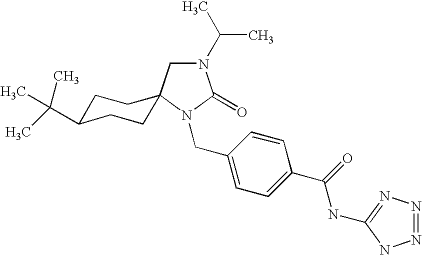 | 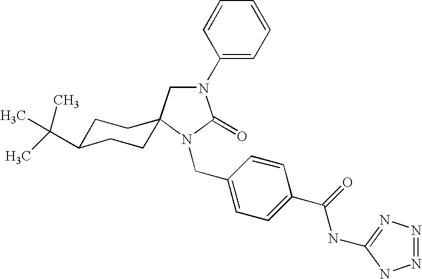 |
| 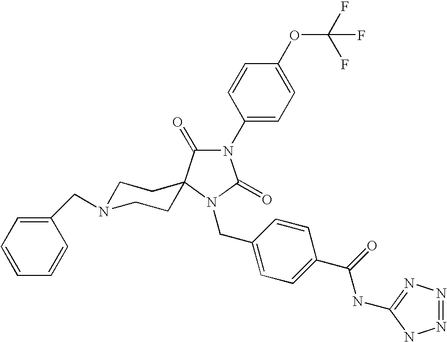 | 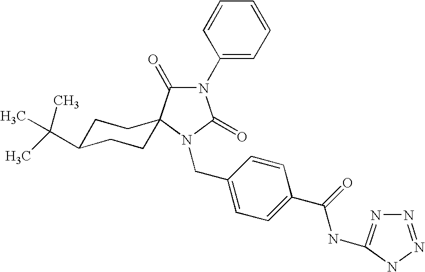 |
| 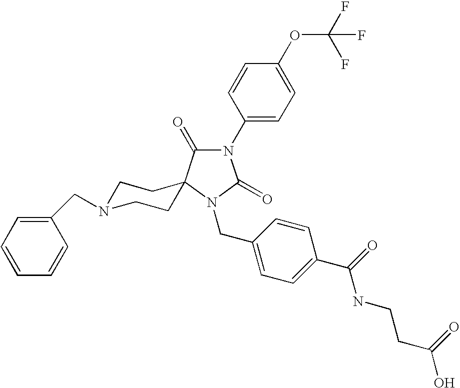 | 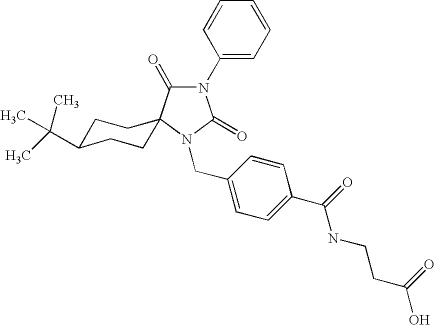 |
| 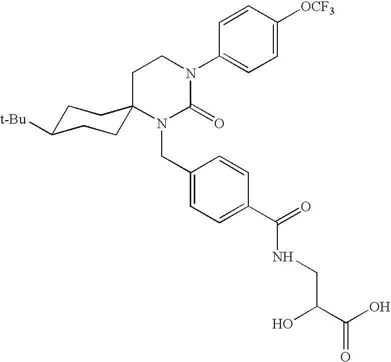 | 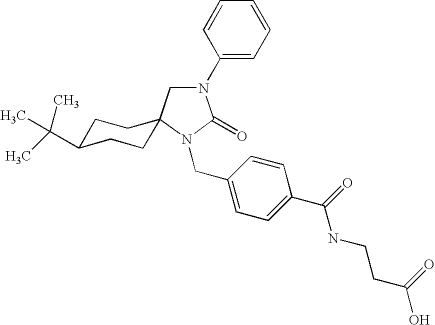 |
| 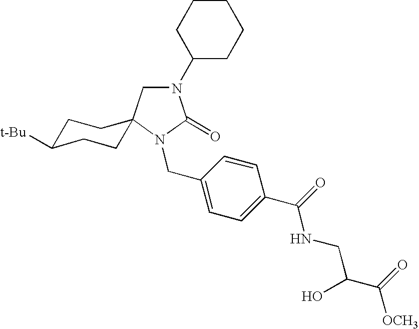 | 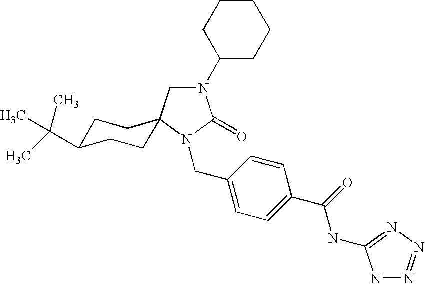 |
| 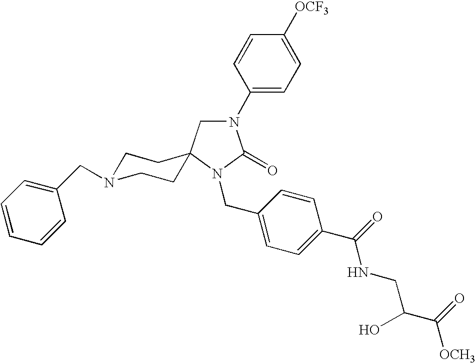 | 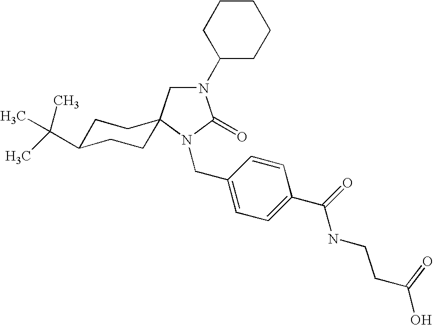 |
| 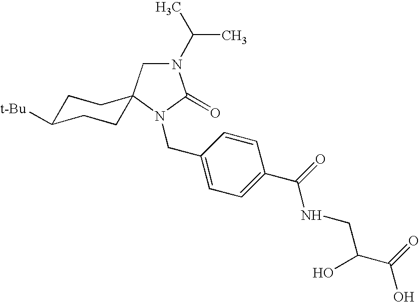 | 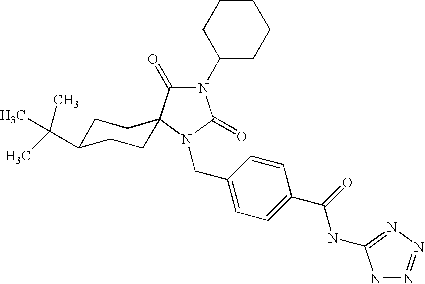 |
| 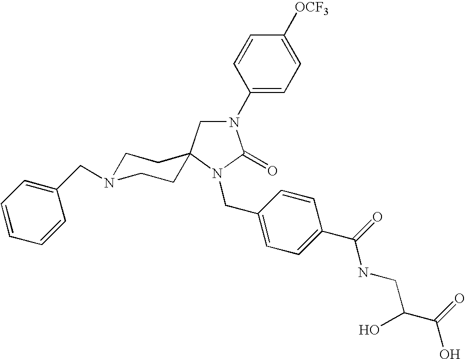 | 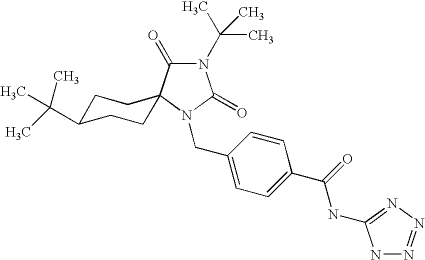 |
| 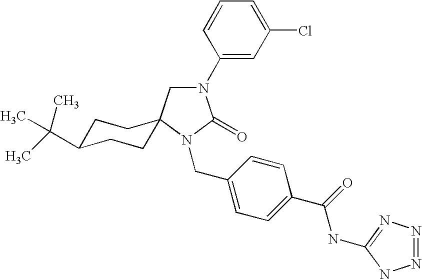 | 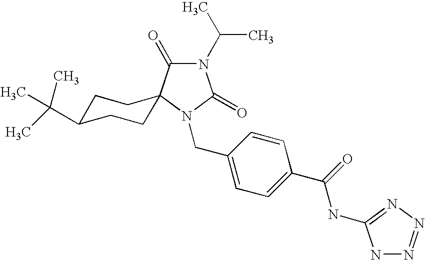 |
| 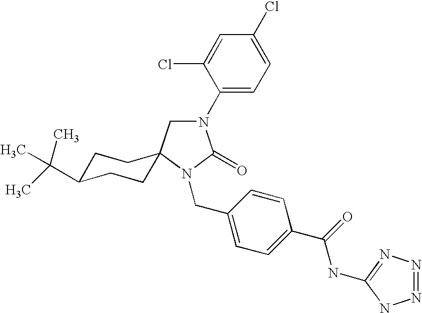 | 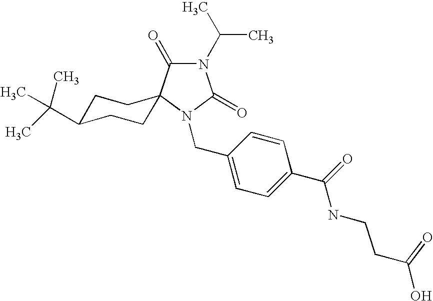 |
| 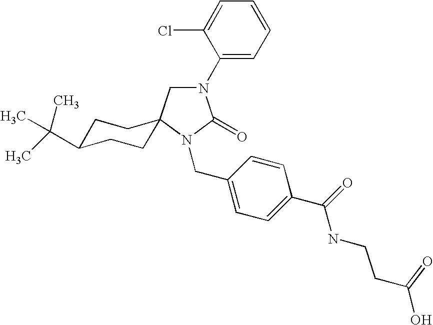 | 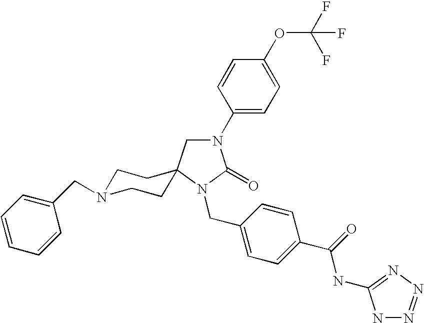 |
| 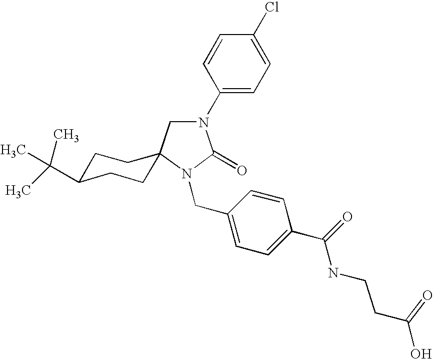 | 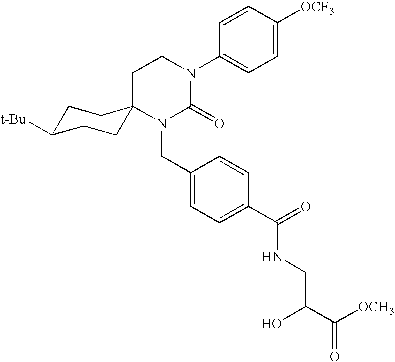 |
| 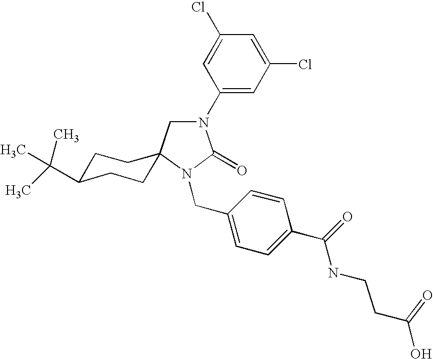 | 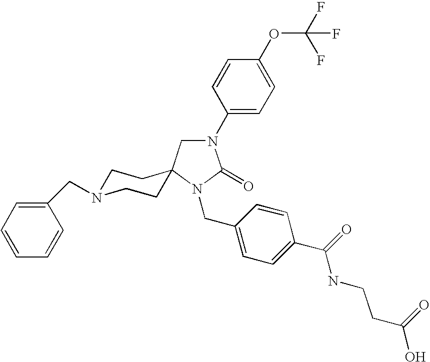 |
| 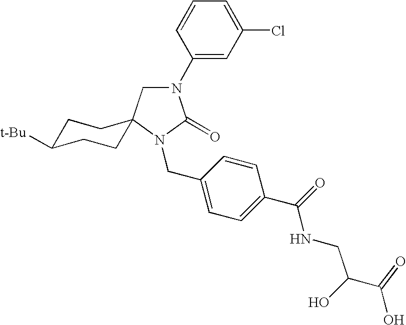 | 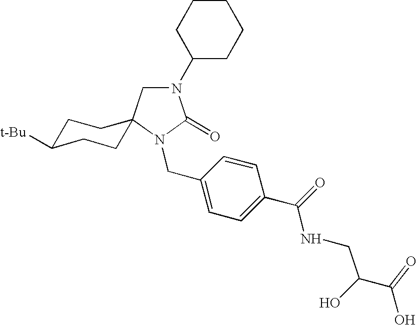 |
| 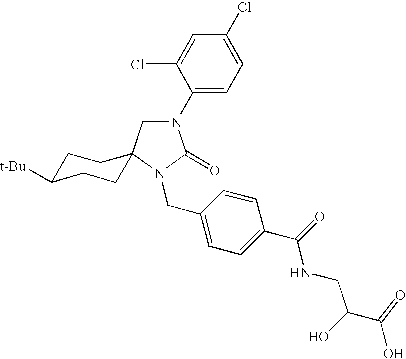 | 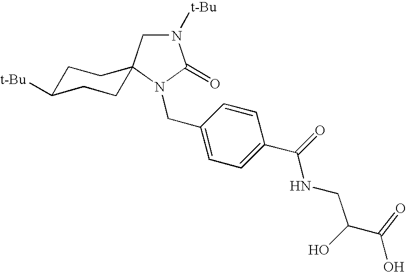 |
| 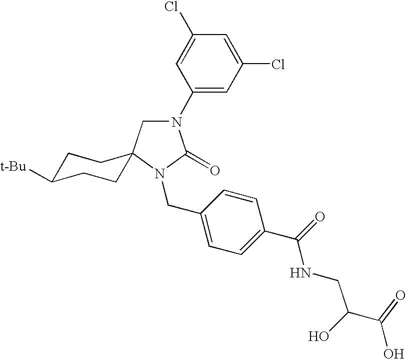 | 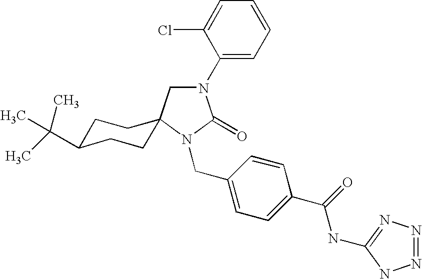 |
| 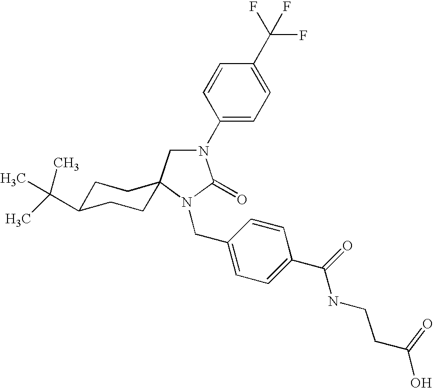 | 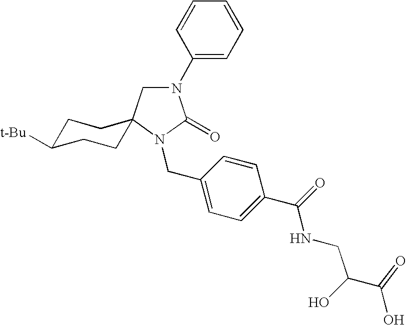 |
| 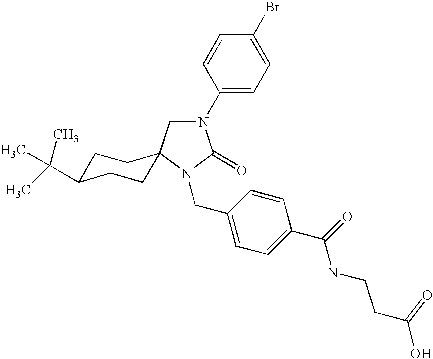 | 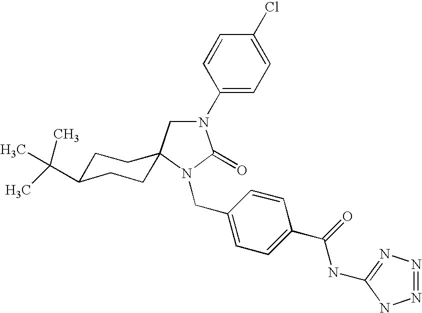 |
| 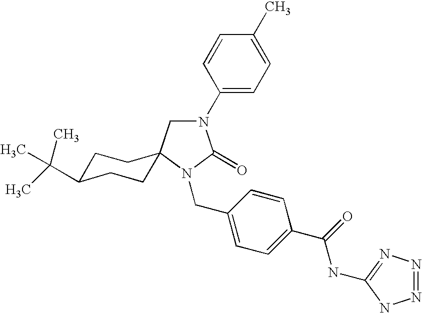 | 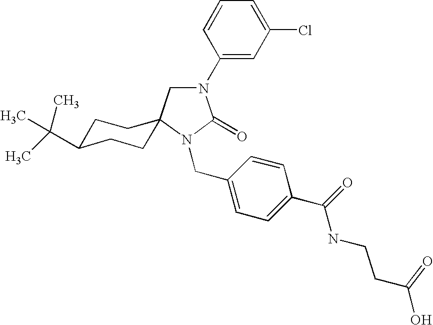 |
| 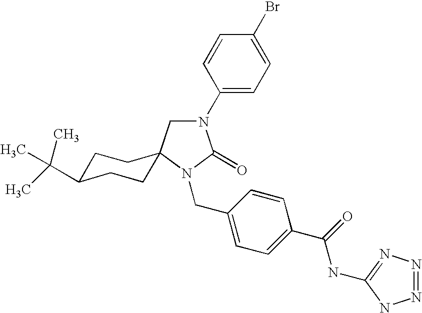 | 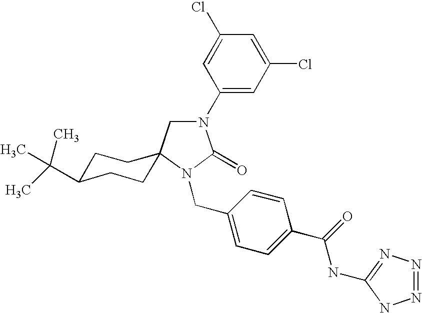 |
| 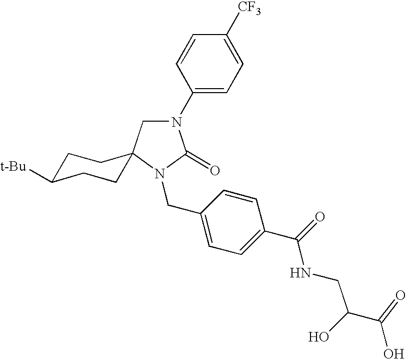 | 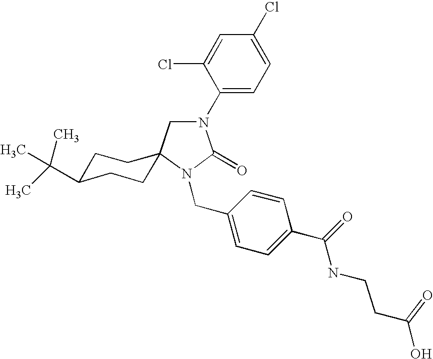 |
| 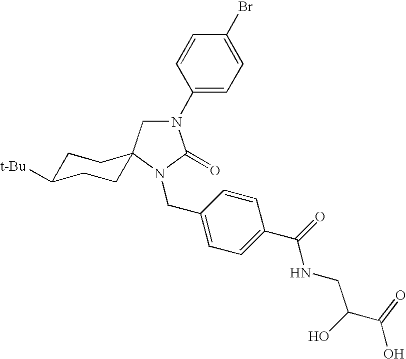 | 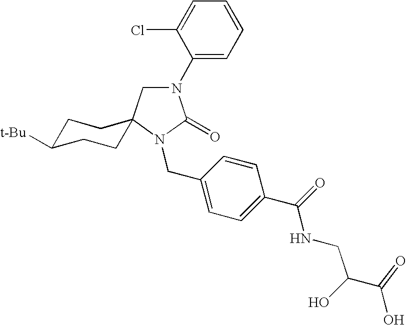 |
| 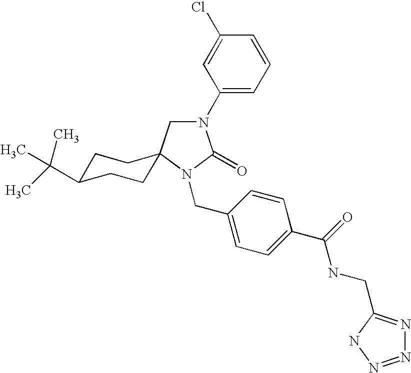 | 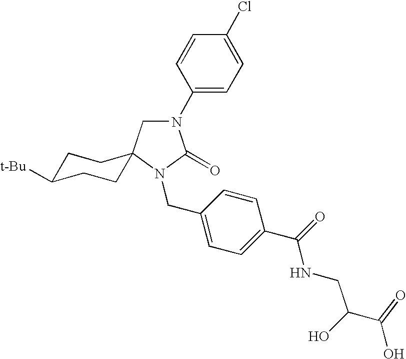 |
| 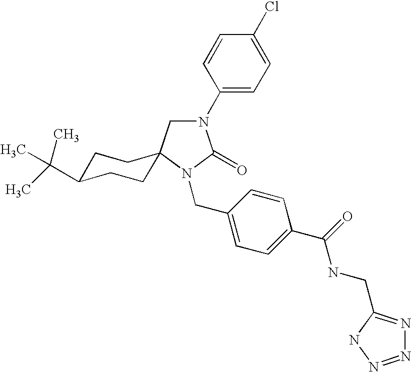 | 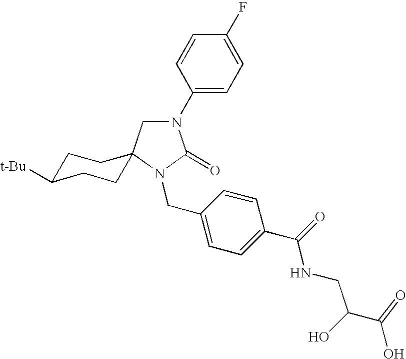 |
| 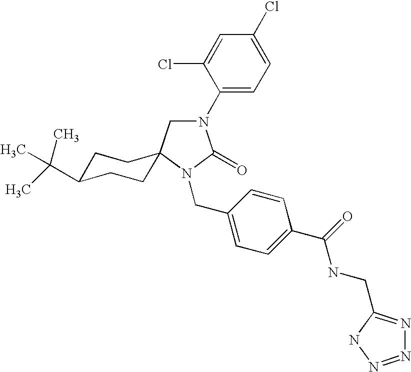 | 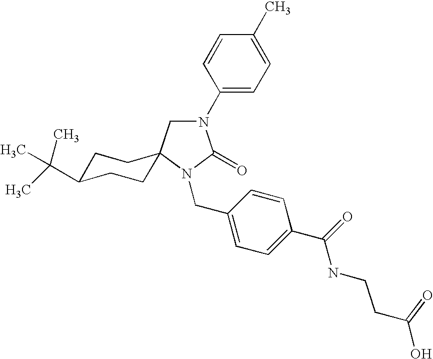 |
| 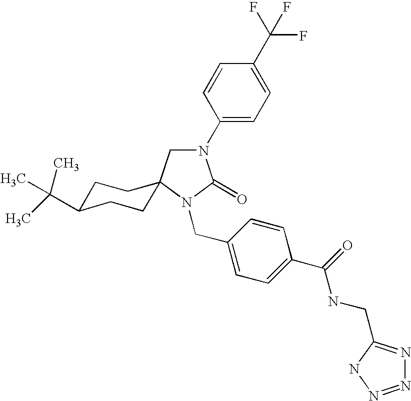 | 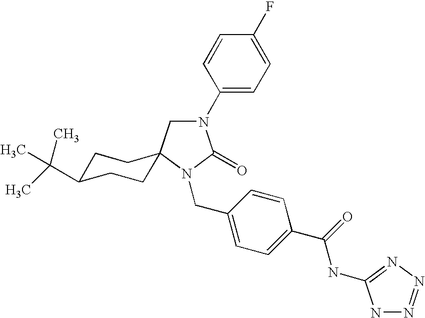 |
| 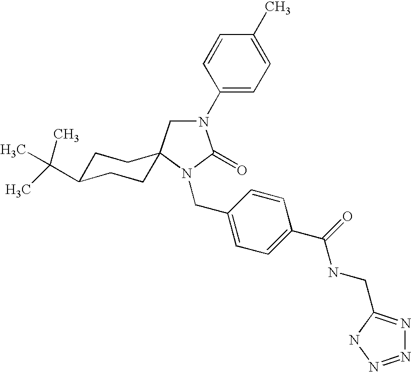 | 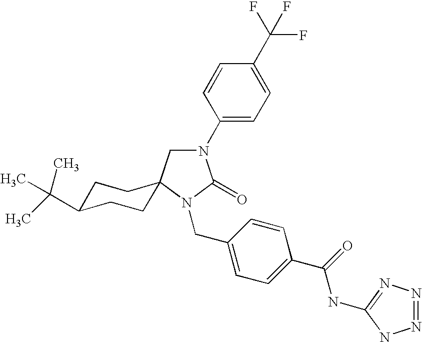 |
| 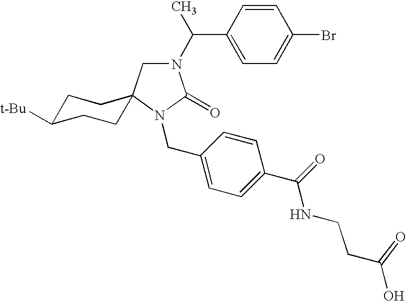 | 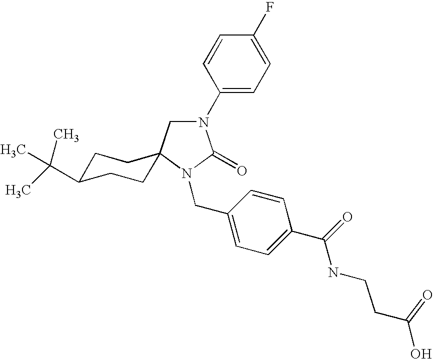 |
| 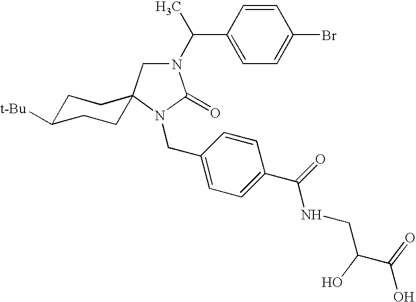 | 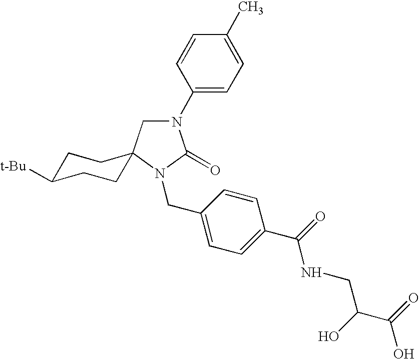 |
| 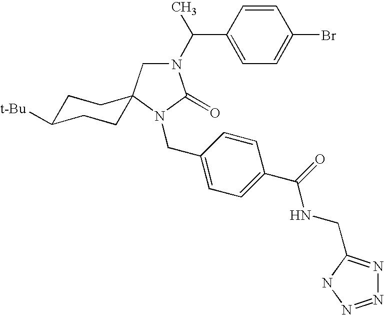 | 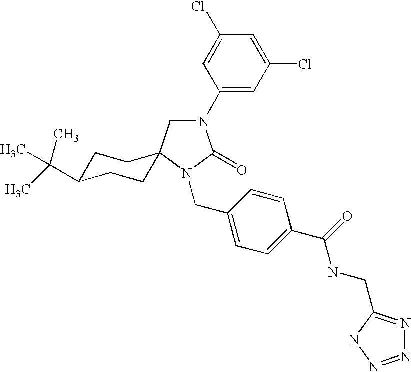 |
| 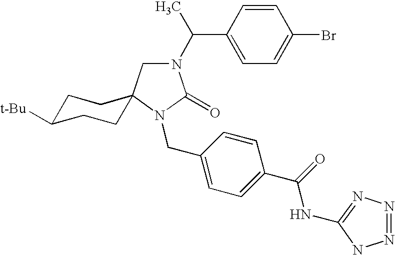 | 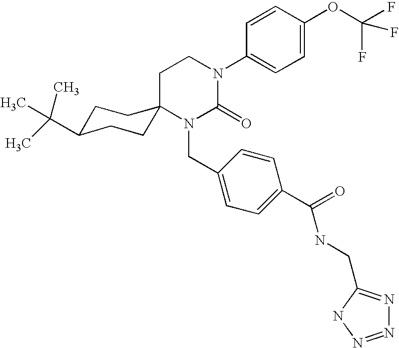 |
| 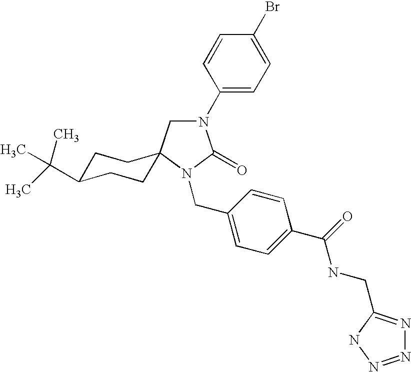 | 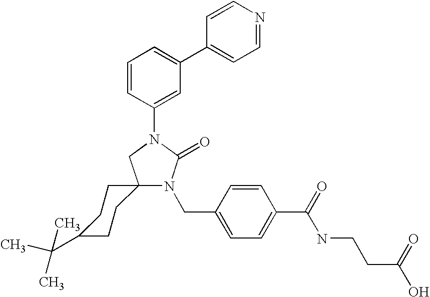 |
| 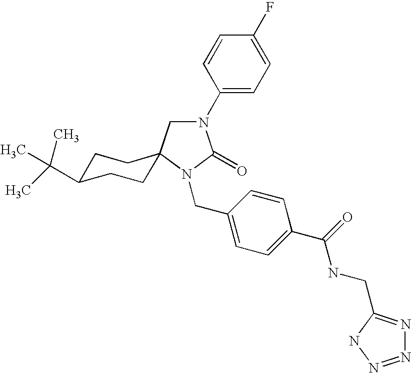 | 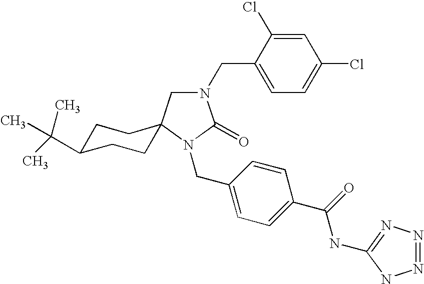 |
| 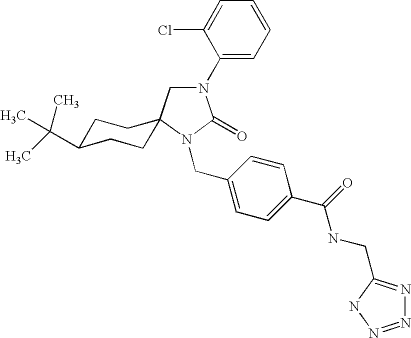 | 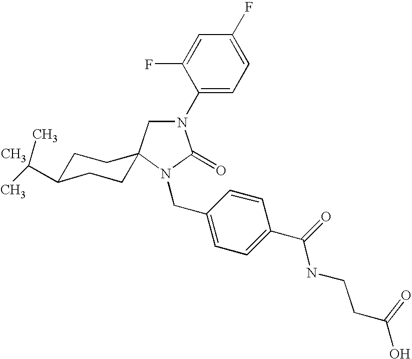 |
| 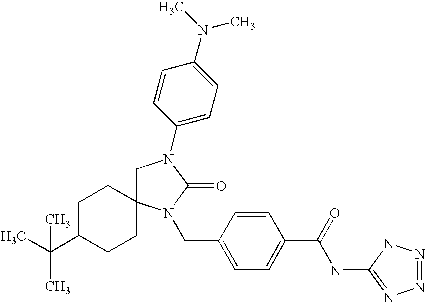 | 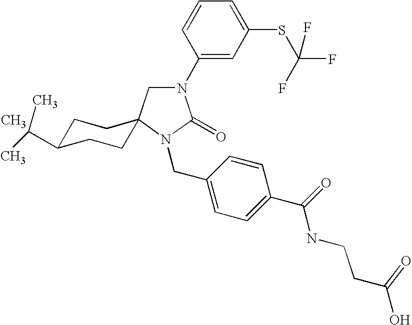 |
| 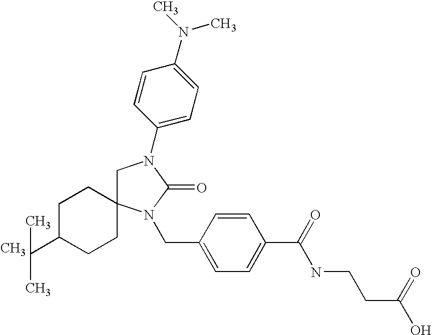 | 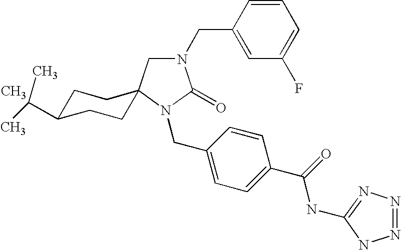 |
| 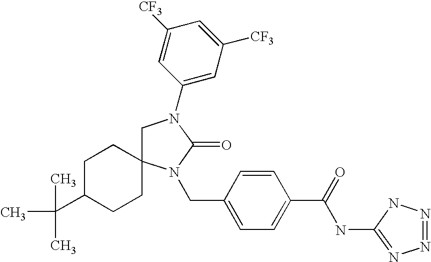 | 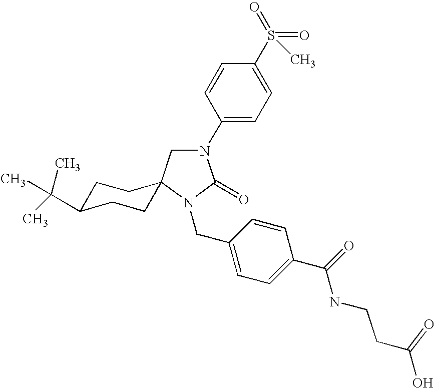 |
| 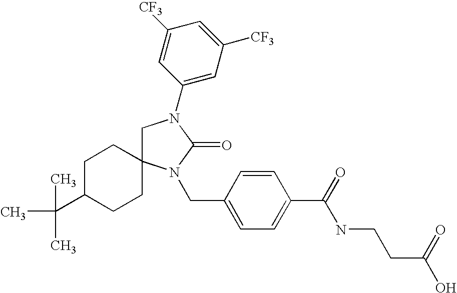 | 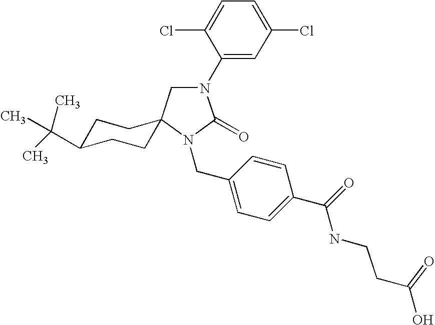 |
| 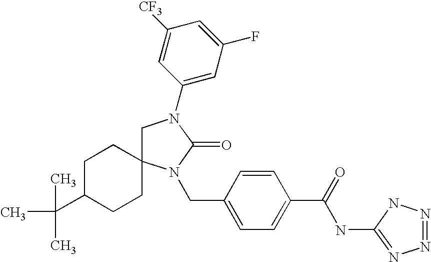 | 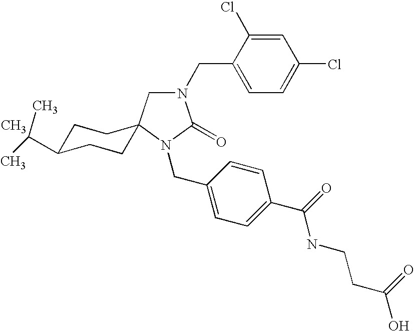 |
| 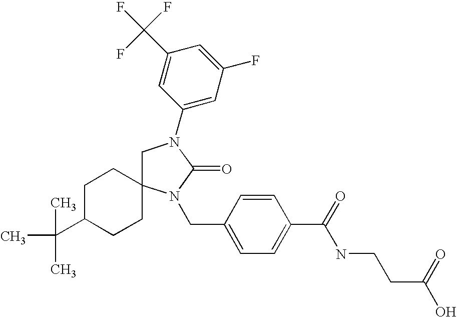 | 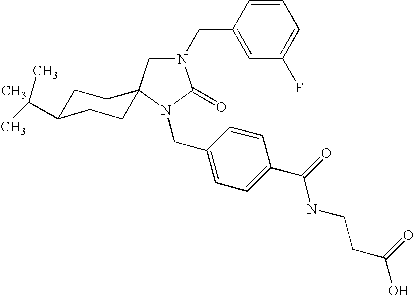 |
| 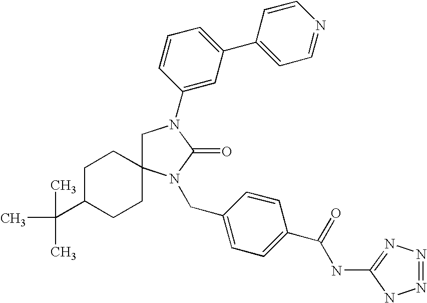 | 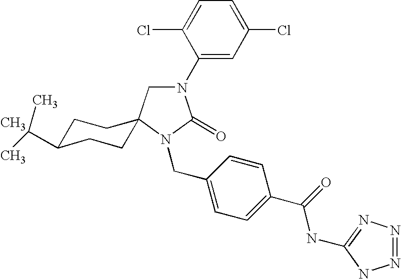 |
| 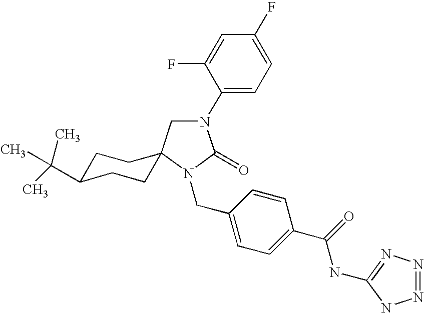 | 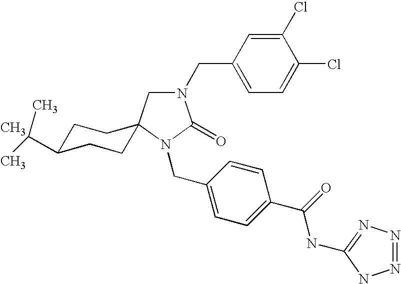 |
| 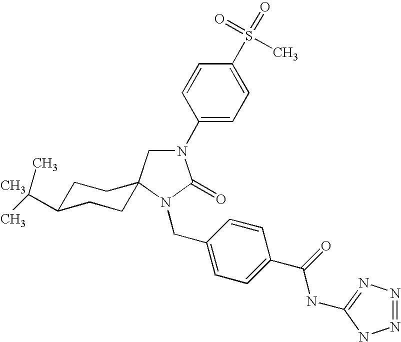 | 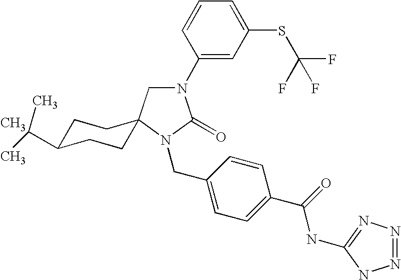 |
| 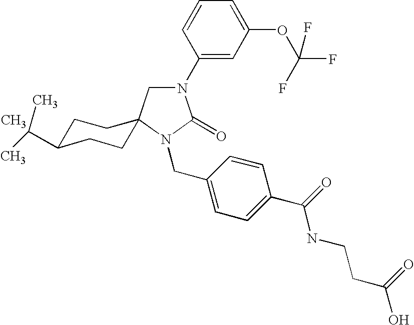 | 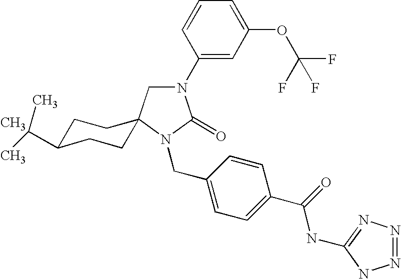 |
| 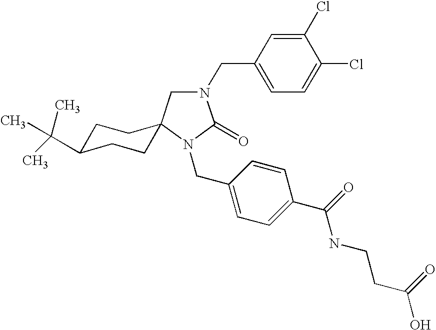 | 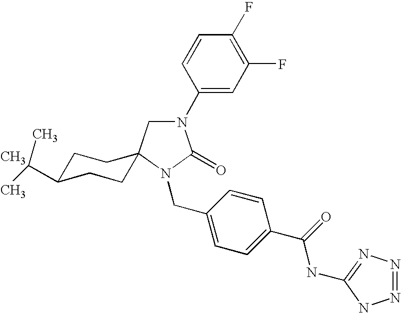 |
| 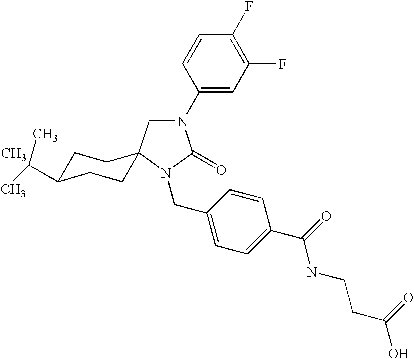 | 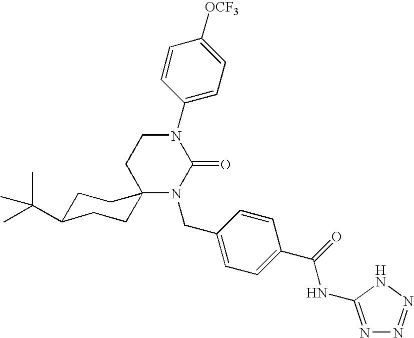 |
| 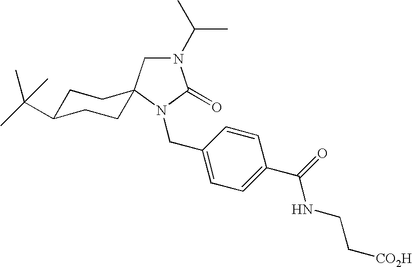 | |

or a pharmaceutically acceptable salt or solvate thereof

20. A pharmaceutical composition comprising a compound in accordance with claim 1 in combination with a pharmaceutically acceptable carrier.

21. A method of treating type 2 diabetes mellitus in a mammalian patient in need of such treatment comprising administering to said patient a compound in accordance with claim 1 in an amount that is effective to treat said type 2 diabetes mellitus.
